(19) United States
(12) Patent Application Publication
(10) Pub. No.: US 2026/0327211 A1
(43) Pub. Date: Oct. 22, 2026

(54) MODULATORS OF A POTASSIUM CHANNEL AND OF TRPV1 CHANNEL AND USES THEREOF

(71) Applicant: Bsense Bio Therapeutics Ltd., Ness Ziona (IL)

(72) Inventors: Haim BELINSON, Even Yehuda (IL); Adi RAVEH, Rehovot (IL); Alan Daniel BROWN, Deal (GB); Alon SILBERMAN, Givatayim (IL)

(73) Assignee: Bsense Bio Therapeutics Ltd., Ness Ziona (IL)

(21) Appl. No.: 19/459,175

(22) Filed: Jan. 26, 2026

Related U.S. Application Data
(63) Continuation of application No. PCT/IL2024/050743, filed on Jul. 26, 2024.
(60) Provisional application No. 63/528,958, filed on Jul. 26, 2023.
(60) Provisional application No. 63/652,726, filed on May 29, 2024.

(51) Int. Cl. C07D 405/14; A61K 31/4439; A61K 31/497; A61K 31/501; A61K 31/506; A61K 31/53; A61P 25/04; C07D 403/04

(52) U.S. Cl. CPC C07D 405/14; A61K 31/4439; A61K 31/497; A61K 31/501; A61K 31/506; A61K 31/53; A61P 25/04; C07D 403/04

(57) ABSTRACT

Compounds represented by Formula I:

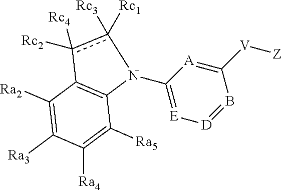

wherein each variable is as defined in the instant specification, and uses thereof in modulating an activity of a voltage-dependent potassium channel and/or a TRPV1 channel and in treating a medical condition associated with an activity of these channels, such as medical conditions associated with neuronal hyper-excitability, are provided.

### RELATED APPLICATIONS

This application is a Continuation of PCT Patent Application No. PCT/IL2024/050743 having International filing date of Jul. 26, 2024 which claims the benefit of priority under 35 USC § 119(e) of U.S. Provisional Patent Application Nos. 63/528,958, filed on Jul. 26, 2023 and 63/652,726, filed on May 29, 2024. The contents of the above applications are all incorporated by reference as if fully set forth herein in their entirety.

### FIELD AND BACKGROUND OF THE INVENTION

The present invention, in some embodiments thereof, relates to therapeutically active compounds and, more particularly, but not exclusively, to newly designed compounds that are derived from diphenylamine analogs, which feature a dual activity as modulators of both Kv7.2/3 and TRPV1 channels, and which are usable in the treatment of medical conditions that are related to these channels, including, but not limited to, medical conditions associated with neuronal hyper-excitability such as pain, tinnitus and pruritus.

Voltage-dependent potassium (Kv) channels conduct potassium ions (K⁺) across cell membranes in response to change in the membrane voltage and thereby can regulate cellular excitability by modulating (increasing or decreasing) the electrical activity of the cell.

Functional Kv channels exist as multimeric structures formed by the association of either identical or dissimilar Kv alpha and/or Kv beta subunits. The alpha subunits comprise six transmembrane domains, a pore-forming loop and a voltage-sensor and are arranged symmetrically around a central pore. The beta or auxiliary subunits interact with the alpha subunits and can modify the properties of the channel complex to include, but not be limited to, alterations in the channel's electrophysiological or biophysical properties, expression levels or expression patterns.

Nine Kv channel alpha subunit families have been identified and are termed Kv1-Kv9. As such, there is an enormous diversity in Kv channel function that arises as a consequence of the multiplicity of sub-families, the formation of both homomeric and heteromeric subunits within sub-families and the additional effects of association with beta subunits [M. J. Christie, Clinical and Experimental Pharmacology and Physiology, 1995, 22 (12), 944-951].

The Kv7 channel family consists of at least five members which include one or more of the following mammalian channels: Kv7.1, Kv7.2, Kv7.3, Kv7.4, Kv7.5 and any mammalian or non-mammalian equivalent or variant (including splice variants) thereof. Alternatively, the members of this family are termed KCNQ1, KCNQ2, KCNQ3, KCNQ4 and KCNQ5, respectively [Dalby-Brown et al., Current Topics in Medicinal Chemistry, 2006, 6, 999-1023].

The five members of this family differ in their expression patterns. The expression of Kv7.1 is restricted to the heart, peripheral epithelial and smooth muscle, whereas the expression of Kv7.2-Kv7.4 is limited to the nervous system to include the hippocampus, cortical neurons and dorsal root ganglion neurons [for a review see, for example, Delmas P. & Brown D., Nature, 2005, 6, 850-862].

The neuronal Kv7 channels have been demonstrated to play key roles in controlling neuronal excitation. Kv7 channels, in particular Kv7.2/Kv7.3 heterodimers, underlie the M-current, a non-activating potassium current found in a number of neuronal cell types. The current has a characteristic time and voltage dependence that results in stabilization of the membrane potential in response to multiple excitatory stimuli. In this way, the M-current is central to controlling neuronal excitability [for a review, see, for example, Delmas. P & Brown. D, Nature, 2005, 6, 850-862].

Potassium channels have been associated with a number of physiological processes, including regulation of heartbeat, dilation of arteries, release of insulin, excitability of nerve cells, and regulation of renal electrolyte transport. Modulators of potassium channels are therefore prime pharmaceutical candidates, and the development of new modulators as therapeutic agents is an on-going research effort.

Thus, given the key physiological role of Kv7 channels in the nervous system and the involvement of these channels in a number of diseases, the development of modulators of Kv7 channels is highly desirable.

Potassium channels modulators are divided to channel-openers and channel-blockers. A potassium channel opener that has gained much attention is retigabine (N-(2-amino-4-(4-fluorobenzylamino)-phenyl)carbamic acid ethyl ester). Retigabine is highly selective for KCNQ2-5-type potassium channels. Use of retigabine for treating neuropathic pain was disclosed in, for example, U.S. Pat. No. 6,117,900 and EP 1223927. Compounds related to retigabine have also been proposed for use as potassium channel modulators (see, for example, U.S. Pat. No. 6,472,165).

However, retigabine has been reported to have multiple effects in neuronal cells. These include sodium and calcium channel blocking activity (Rundfeldt, C, 1995, Naunyn-Schmiederberg's Arch Pharmacol, 351 (Suppl): R160) and effects on GABA (γ-aminobutyric acid) synthesis and transmission in rat neurons (Kapetanovic, I. M., 1995, Epilepsy Research, 22, 167-173, Rundfeldt, C, 1995, Naunyn-Schmiederberg's Arch Pharmacol, 351 (Suppl):R160).

Other KCNQ potassium channel modulators have been described in, for example, U.S. patent application Ser. No. 10/075,521, which teaches 2,4-disubstituted pyrimidine-5-carboxamide derivatives as Kv7 modulator; U.S. patent application Ser. No. 10/160,582, which teaches cinnamide derivatives as voltage-dependent potassium channel modulators; U.S. Pat. No. 5,565,483 and U.S. patent application Ser. Nos. 10/312,123, 10/075,703 and 10/075,522, which teach 3-substituted oxindole derivatives as voltage-dependent potassium channel modulators; U.S. Pat. No. 5,384,330, which teaches 1,2,4-triamino-benzene derivatives as potassium channel modulators; and U.S. Pat. No. 6,593,349 which teaches bisarylamines derivatives as voltage-dependent potassium channel modulators. U.S. Pat. No. 6,291,442 teaches compounds comprising two or three aromatic rings having a free carboxyl or a carboxyl being linked, via an ester bond, to a lower alkyl ester, attached to one of the rings, for the modulation of Shaker class of voltage gated potassium channels.

WO 2004/035037 and U.S. Patent Application Publication No. 20050250833 teach derivatives of N-phenylanthranilic acid and of 2-benzimidazolone as potassium channel openers, especially openers of voltage-dependent potassium channels such as Kv7.2, Kv7.3 and Kv7.2/7.3 channels, as well as neuron activity modulators.

WO 2009/037707 teaches additional derivatives of N-phenylanthranilic as potassium channel and/or TRPV1 modulators. An exemplary modulator disclosed in WO 2009/037707 is referred to as NH29:

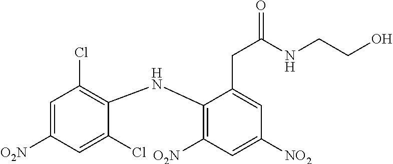 [SMILES: O=C(Cc1cc([N+](=O)[O-])cc([N+](=O)[O-])c1Nc1c(Cl)cc([N+](=O)[O-])cc1Cl)NCCO]

WO 2009/071947 and WO 2010/010380 teach derivatives of diphenylamine as potassium channel modulators. Exemplary modulators disclosed in these patent applications are referred to as NH34 and NH43:

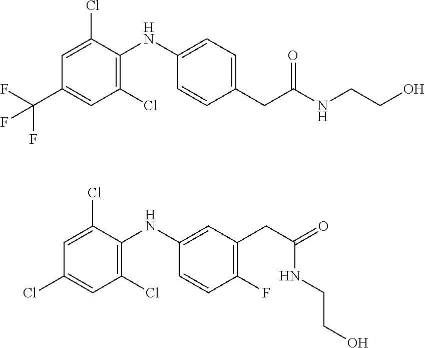 [SMILES: O=C(Cc1ccc(Nc2c(Cl)cc(C(F)(F)F)cc2Cl)cc1)NCCO ; O=C(Cc1cc(Nc2c(Cl)cc(Cl)cc2Cl)ccc1F)NCCO]

Transient receptor potential vanilloid type 1 (TRPV1) receptor is a ligand-gated non-selective cation channel activated by heat (typically above 43° C.), low pH (<6) and endogenous lipid molecules such as anandamide, N-arachidonoyl-dopamine, N-acyl-dopamines and products of lipoxygenases (e.g., 12- and 15-(S)-HPETE) termed endovanilloids. Apart from peripheral primary afferent neurons and dorsal root ganglia, TRPV1 receptor is expressed throughout the brain. Recent evidence shows that TRPV1 receptor stimulation by endocannabinoids or by capsaicin leads to analgesia and this effect is associated with glutamate increase and the activation of OFF cell population in the rostral ventromedial medulla (RVM).

TRPV1 has also been found to be involved in the regulation of body temperature, anxiety and mediation of long-term depression (LTD) in the hippocampus. TRPV1 channels are also located on sensory afferents, which innervate the bladder. Inhibition of TRPV1 has been shown to ameliorate urinary incontinence symptoms.

TRPV1 modulators have been described in, for example, WO 2007/054480, which teaches the effect of 2-(benzimidazol-1-yl)-acetamide derivatives in the treatment of TRPV1 related diseases. WO 2008/079683 teaches compounds being a conjugated two-ring system of cyclohexyl and phenyl for inhibiting TRPV1 receptor. EP 01939173 teaches O-substituted-dibenzyl urea- or thiourea-derivatives as TRPV1 receptor antagonists. WO 2008/076752 teaches benzimidazole compounds as potent TRPV1 modulators and EP 01908753 teaches TRPV1 modulators being heterocyclidene acetamide derivatives.

The potassium channel Kv7.2/3 and the cation non-selective channel TRPV1 are uniquely co-expressed in afferent peripheral sensory neurons (DRG sensory neurons), which convey sensory signals and have opposite functions. TRPV1 channels trigger the pain signals, while Kv7.2/3 channels inhibit them. Compounds that simultaneously function as activators (e.g., openers) of Kv7.2 and inhibitors (e.g., blockers) of TRPV1 can depress neuronal hyper-excitability associated with medical conditions such as pain, tinnitus and pruritus.

WO 2019/073471 discloses various modifications performed to the structures taught in WO 2009/071947 and WO 2010/010380, which were found to exhibit dual modulation of both a voltage-dependent potassium channel and of TRPV1. Two of the potential candidates disclosed in WO 2019/073471 are referred to therein as NH91 and NH101:

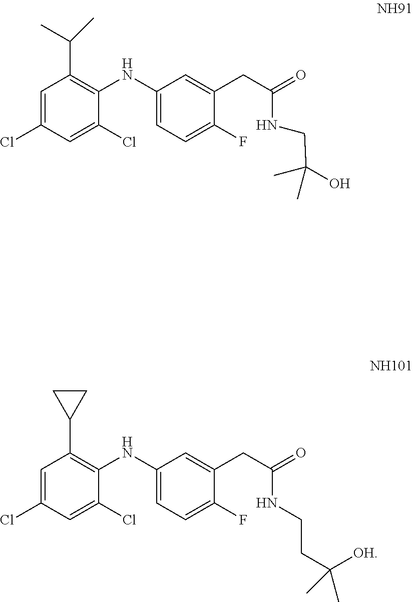 [SMILES: CC(C)c1cc(Cl)cc(Cl)c1Nc1ccc(F)c(CC(=O)NCC(C)(C)O)c1 ; CC(C)(O)CCNC(=O)Cc1cc(Nc2c(Cl)cc(Cl)cc2C2CC2)ccc1F]

Additional Background Art includes WO 2023/139581.

### SUMMARY OF THE INVENTION

According to an aspect of some embodiments of the present invention there is provided a compound represented by Formula I:

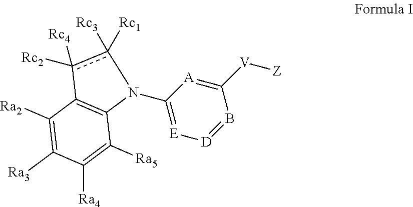

wherein:
A is C—Rb1 or N (nitrogen atom);
B is C—Rb2 or N (nitrogen atom);
D is C—Rb3 or N (nitrogen atom);
E is C—Rb4 or N (nitrogen atom);
Ra2-Ra5 are each independently hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of Ra2-Ra5 form together an alicyclic or aromatic ring;
Rb1, Rb2 and Rb4 are each independently hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Rb substituents form together an alicyclic, heteroalicyclic, aromatic or heteroaromatic ring;
Rc1-Rc4 are each independently absent, hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Rc substituents form together an alicyclic or heterocyclic ring,
The dashed line represents a single bond (when absent) or a double bond (when present), such that when the dashed line represents a double bond, Rc3 and Rc4 are absent;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof, and
Z is represented by Formula II:

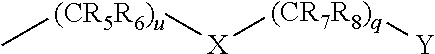

wherein:
u and q are each independently an integer of from 0 to 4, provided that u+q is at least 2;
X is selected from —O— and —NR₉—, or is absent;
Y is a polar hydrophilic group such as OR₁₁, SR₁₁, amine (NR₁₂R₁₃), or amide (—NR₁₂—C(═O)—R₁₄;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring; and
R₉, R₁₀, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring.

According to some of any of the embodiments described herein, at least one of A, B, D and E is N (nitrogen).

According to some of any of the embodiments described herein, at least two of A, B, D and E is N (nitrogen).

According to some of any of the embodiments described herein, each of Rc1-Rc4 is independently selected from hydrogen, alkyl, haloalkyl, and halo.

According to some of any of the embodiments described herein, the dashed line represents a double bond.

According to some of any of the embodiments described herein, Rc1 and Rc2 are each independently selected from hydrogen, alkyl, haloalkyl, and halo.

According to some of any of the embodiments described herein, Rc1 is hydrogen.

According to some of any of the embodiments described herein, Rc2 is selected from halo and haloalkyl and is preferably halo.

According to some of any of the embodiments described herein, at least two of the Ra substituents are selected from halo and alkoxy.

According to some of any of the embodiments described herein, Ra3 and Ra5 are each independently a halo (e.g., chloro).

According to some of any of the embodiments described herein, at least one of A and B is N (nitrogen).

According to some of any of the embodiments described herein, A and B are each N (nitrogen).

According to some of any of the embodiments described herein, E and B are each N (nitrogen).

According to some of any of the embodiments described herein, X is absent.

According to some of any of the embodiments described herein, at least one of R₅, R₆, R₇ and R₈ is independently selected from alkyl, hydroxy, alkoxy, haloalkyl, ether, and halo, and/or at least two of R₅, R₆, R₇ and R₈ form together an alicyclic or heteroalicyclic ring.

According to some of any of the embodiments described herein, u is 1 or 2.

According to some of any of the embodiments described herein, q is 1 and at least one or each of R₇ and R₈ is alkyl or at least one or each of R₇ and R₈ is hydrogen.

According to some of any of the embodiments described herein, at least one of R₅, R₆, R₇ and R₈ is independently hydroxy, hydroxyalkyl, ether, or a heteroalicyclic (e.g., oxygen-containing).

According to some of any of the embodiments described herein, at least two of R₅, R₆, R₇ and R₈ form together an alicyclic ring or a heteroalicyclic ring, preferably an oxygen-containing 4, 5 or 6-membered heteroalicyclic.

According to some of any of the embodiments described herein, X is absent, u is 1 or 2, q is 1 and R₅, R₆, R₇ and R₈ form together a cyclobutane or tetrahydrofuran.

According to some of any of the embodiments described herein, X is absent, u is 1 or 2, q is 1 and each of R₅, R₆, R₇ and R₈ is hydrogen.

According to some of any of the embodiments described herein, Y is OR₁₁, and R₁₁ is hydrogen or an alkyl, preferably substituted by at least one hydroxy, amide and/or carboxy or a heteroaryl or heteroalicyclic.

According to some of any of the embodiments described herein, Y is OR₁₁, and R₁₁ is hydrogen.

According to some of any of the embodiments described herein, the compound is selected from the compounds presented in FIG. 17.

According to some of exemplary embodiments, the compound is Compound 760:

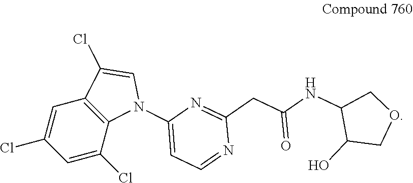 [SMILES: O=C(Cc1nccc(-n2cc(Cl)c3cc(Cl)cc(Cl)c32)n1)NC1COCC1O]

According to exemplary embodiments, the compound is Compound 767 (see, FIG. 17).

It is to be noted that the compounds as presented herein throughout encompass all possible isomers and stereoisomers, also when a specific stereoconfiguration is shown. Thus, Compound 760 is alternatively presented as a specific stereoisomer, as in FIG. 17, and encompasses this stereoconfiguration as well as other stereoconfigurations.

According to some of any of the embodiments described herein, the compound is characterized by at least one of:
Log D, determined as described herein, lower than 4;
Ligand-lipophilicity efficiency (LLE), determined as described herein, higher than 3, or higher than 5;
HLM Clint, determined as described herein, lower than 100 or lower than 60 ml/min/kg;
Kinetic solubility higher than 20 or higher than 30 or higher than 40 micromolar;
Caco-2 Efflux ratio, determined as described herein, lower than 3; and
A time-dependent inhibition (TDI) of CYP3A4 enzyme in the presence of midazolam, determined by the (−)NADPH/(+)NADPH ratio as described herein, lower than 1.57.

According to an aspect of some embodiments of the present invention there is provided a pharmaceutical composition comprising the compound of Formula I as described herein in any of the respective embodiments and any combination thereof and a pharmaceutically acceptable carrier.

According to an aspect of some embodiments of the present invention there is provided a compound or a pharmaceutical composition, as described herein in any of the respective embodiments and any combination thereof for use in modulating an activity of a voltage-dependent potassium channel.

According to an aspect of some embodiments of the present invention there is provided a compound or a pharmaceutical composition, as described herein in any of the respective embodiments and any combination thereof for use in modulating an activity of TRPV1.

According to an aspect of some embodiments of the present invention there is provided a compound or a pharmaceutical composition, as described herein in any of the respective embodiments and any combination thereof for use in modulating an activity of both a voltage-dependent potassium channel and TRPV1.

According to an aspect of some embodiments of the present invention there is provided a method of modulating an activity of a voltage-dependent potassium channel and/or of TRPV1, the method comprising contacting cells expressing a respective channel or channels with a compound or a pharmaceutical composition, as described herein in any of the respective embodiments and any combination thereof.

According to an aspect of some embodiments of the present invention there is provided a method of modulating an activity of a voltage-dependent potassium channel and/or of TRPV1 in a subject in need thereof, the method comprising administering to the subject a compound or a pharmaceutical composition, as described herein in any of the respective embodiments and any combination thereof.

According to some of any of the embodiments described herein, modulating the activity of the voltage-dependent potassium channel comprises opening the channel and wherein modulating the activity of the TRPV1 channel comprises inhibiting an activity of the channel.

According to some of any of the embodiments described herein, the potassium channel is Kv7.2/7.3.

According to an aspect of some embodiments of the present invention there is provided a compound or a pharmaceutical composition, as described herein in any of the respective embodiments and any combination thereof, for use in treating a medical condition associated with an activity of a voltage-dependent potassium channel and/or a TRPV1 channel.

According to some of any of the embodiments described herein, the medical condition is neuropathic pain, pruritus or tinnitus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the chemical structures of exemplary compounds encompassed by chemotype 1 according to some embodiments of the present invention.

FIG. 2 presents the chemical structures of exemplary compounds encompassed by chemotype 2 according to some embodiments of the present invention.

FIGS. 3A-B present the chemical structures of exemplary compounds encompassed by chemotypes 3 and 4 according to some embodiments of the present invention (FIG. 3A) and the potential enzyme-catalyzed transformation therebetween (FIG. 3B).

FIGS. 4A-B present a homology model of the human TRPV1 with reference (previously described) compounds docked into the vanilloid pocket: Reference compounds NH91 (000091; bright yellow), 000228 (as disclosed, for example, in WO 2004/035037; green) and the TRPV1 established agonist resiniferatoxin (RTX) serving as a reference molecule (FIG. 4A); and a homology model of the human Kv7.2/7.3 with the previously described 000091 (NH91) docked inside the VSD (FIG. 4B). Hydrophobic gaskets and hydrophilic channels are shared structural motifs of both proteins allowing a design dual Kv7.2/7.3 and TRPV1 modulators.

FIGS. 5A-B present a representative example of TRPV1 qSAR modelling. FIG. 5A presents a superposition of exemplary potent TRPV1 inhibitors according to some embodiments of the present invention. FIG. 5B is a linear plot correlating experimental IC50 results (X axis) and the predicted qSAR IC50 values for exemplary compounds according to some embodiments of the present invention.

FIG. 6A-C present data obtained for rat DRG neuron firing in responses to current injection (FIG. 6A), Capsaicin (FIG. 6B) or both (FIG. 6C) activation with or without application of varying concentrations of AMG9810—a TRPV1 specific antagonist, compound 273—a Kv activator lacking TRPV1 inhibition, and exemplary compound 421-6—showing an exemplary Kv and TRPV1 dual targeting compound and Retigabine (RET) as an exemplary Kv7.2/3 targeting compound with similar EC50 to that of 421-6.

FIGS. 6D-E present dose response curves displaying compound 421-6 and retigabine inhibition (FIGS. 6D and 6E, respectively) of both capsaicin-induced and current-induced neuronal excitability at varying concentrations.

FIGS. 7A-C present exemplary data obtained for rat DRG neuron firing in response to current injection and Capsaicin firing response of compound 552 at 100 nM (FIG. 7A); a bar graph showing comparative rat DRG firing responses between compounds 421-6, 541, 552 and 533 (racemic), each at 100 nM; and exemplary data obtained for rat DRG neuron firing in responses to current injection and Capsaicin firing response in the presence of compound 533 (racemic mixture) at 100 nM.

FIG. 8A (Background Art) presents an experimental set up according to Zhang et al., 2005, EMBO J 24(24):4211-23 as described herein.

FIG. 8B presents representative data obtained in assays in which neurons were pretreated with NGF 100 ng/ml for 4-5 days. NGF Non-treated rat DRGs were stimulated by repeated capsaicin application with and without 421-6 at 2 μM (top panel) or 100 nM (Middle panel). Following NGF pretreatment tetanic burst of firing is observed and a reversible inhibition of 421-6 at 100 nM is shown.

FIGS. 8C-D present magnifications of the data presented in FIG. 8B (bottom panel (FIG. 8C), and data obtained following NGF pretreatment in the presence of compound 421-6 (100 nM) and compound 533 at 2 μM (FIG. 8D).

FIGS. 9A-B demonstrate the inhibition potency of compound 415 (5 μM, 1.5 minute; FIG. 9A) and of previously described compound 291 (2 μM,) and compound 414 (5 μM) (FIG. 9B) on neuronal spontaneous activity in human derived sensory neurons, displaying both the high potency of the compound together with its reversible manner, with recovered firing following compound's wash away.

FIGS. 10A-D present data showing the effect of compound 533 as a racemic mixture (533R) and of its separated enantiomers, 533p1 and 533p2. FIG. 10A presents comparative plots showing hKv7.2/3 activation measured using high-content fluorescent assay, comparing hKv7.2/3 activation by 533 racemic mixture (533R) to its separated enantiomers, 533p1 and 533p2, showing lower and higher activation potencies, respectively. FIG. 10B presents comparative plots showing that each of the 533 separated enantiomers regulates differently hTRPV1, one (533p1) inhibits hTRPV1 while the other (533p2) activates hTRPV1, with 533 racemic mixture (533R) showing an averaged response. AMG9810, a hTRPV1 antagonist served as a positive control. FIG. 10C presents the EC/IC50 values (in μM) of each of the separated enantiomers of compound 533 against the hKv7.2/3 and hTRPV1 targets (arrow denotes TRPV1 activation). FIG. 10D presents data showing that 533p2 application (100 nM) inhibits the current-evoked rat DRGs spiking, while the capsaicin-evoked rat DRGs spiking is facilitated, in agreement with 533p2 TRPV1 activation property (upper panel), whereby when 533 racemic mixture (533R) is applied, current-evoked spiking inhibition decreases, while capsaicin-induced spiking inhibition is gained.

FIGS. 11A-E present comparative plots showing the caspain-induced inhibition of hTRPV1 activity, measured using fluorescent assay, by compound 627 and AMG9810 as a positive control in hTRPV1 stably expressing cells (FIG. 11A), and in cells expressing hKv7.2/3 and hTRPV1 (FIG. 11B); comparative plots showing activation of hKv7.2/3 activity, measured using fluorescent assay, by compound 627 compared to retigabine positive control (FIG. 11C), and activation of hKv7.2/3 activity, measured using fluorescent assay, by compound 627 at very low concentrations (at the picomolar range) (FIG. 11D); and activation of hKv7.2/3 activity by compound 627 in electrophysiology, measuring whole-cell currents of hKv7.2/3 expressed in CHO cells, clamping membrane potential to −40 mV (1.5 s) (from a holding potential of −90 mV) (N=4) (FIG. 11E).

FIGS. 12A-C present whole-cell currents electrophysiology measurements (FIG. 12A) in hTRPV1 expressing CHO cells (upper panel) and in CHO cells co-expressing hKv7.2/3 and hTRPV1 upon activation (lower panel); a dose-response curve displaying the hTRPV1 inhibition-potency in CHO cells expressing hTRPV1 (right) and co-expressing with hKv7.2/3 and activated (left) (FIG. 12B); and an exemplary capsaicin-induced current in CHO cells co-expressing hTRPV1 and hKv7.2/3 without hKv7.2/3 activation, in the presence of compound 627 (FIG. 12C).

FIGS. 13A-B present exemplary capsaicin-induced current in CHO cells co-expressing hTRPV1 and hKv7.2/3 in the presence of AMG9810, a known TRPV1 inhibitor (FIG. 13A) and dose-response comparative plots displaying the hTRPV1 inhibition-potency in CHO cells expressing hTRPV1 and cells co-expressing hTRPV1 and hKv7.2/3 and activated, in the presence of compound 627, AMG9810, and AMG9810 and retigabine (RET) at 1 μM (FIG. 13B).

FIG. 14 presents rat DRG membrane-potential recording showing action-potentials trains in response to Capsaicin application in the presence of compound 627 at 1 nM (upper panel) and 0.1 nM (lower panel)

FIGS. 15A-J present whole-cell currents electrophysiology measurements (FIG. 15A) in CHO cells co-expressing hKv7.2/3 and hTRPV1 following hKv7.2/3 activation, in the presence of compound 661, 0.1 nM (upper panel) or 0.01 nM (lower panel), respectively (FIG. 15A); Dose-response plots displaying the hTRPV1 inhibition-potency in CHO cells co-expressing hKv7.2/3 and hTRPV1 following hKv7.2/3 activation, in the presence of varying concentrations of compound 661, compound 627, or AMG9810 (FIG. 15B); Data obtained for rat DRG neuron firing in response to Capsaicin application with exemplary recording using compound 661 at 0.001 nM, showing approximately 50% inhibition of action potential firing (FIG. 15C); Comparative plots showing the effect on Kv7.2/3 activity, measured in cells expressing only Kv7.2/3 using fluorescent assay, by compound BS661 compared to retigabine positive control (FIG. 15D); Comparative plots showing the effect on TRPV1 activity, measured in cells expressing only TRPV1 using fluorescent assay, by compound 661 (denoted BS661) compared to AMG9810 positive control (FIG. 15E); Exemplary capsaicin-induced current in CHO cells co-expressing hTRPV1 and hKv7.2/3 and activated in the presence of compound 661 (denoted BS661) (FIG. 15F); Comparative plots showing the effect on hKv7.3/5 activity, measured in cells expressing Kv7.3/5 using fluorescent assay, by compound 661 (denoted BS661) compared to retigabine positive control (FIG. 15G); Comparative plots showing the effect on hKv7.4 activity, measured in cells expressing Kv7.4 using fluorescent assay, by compound 661 (denoted BS661) compared to retigabine (denoted RET) positive control (FIG. 15H); Comparative plots showing the effect on hERG activity, measured in cells expressing hERG using fluorescent assay, by compound 661 (denoted BS661) compared to terfenladine (denoted TERF) positive control (FIG. 15I); and Comparative plots showing the effect on hepG2 viability, measured using fluorescent assay, by compound 661 (denoted BS661) compared to TAK243 (denoted TAK) positive control (FIG. 15J).

FIGS. 16A-Q present rat PK exposure of compound 661 (denoted BS661) following oral delivery of 10 mg/kg (FIG. 16A); Rat PK exposure of compound 661 following oral delivery of 10 mg/kg, extrapolated to 0.2, 2 and 20 mg/kg (FIG. 16B); an efficacy profile of compound 661 in the Spared Nerve Injury (SNI) model at doses of 0.2, 2 and 20 mg/kg compared to pregabalin (30 mg/kg) (FIG. 16C); a bar graph showing the time spent on rotarod (% from pre-dose) at 1 hour and 4 hour post-dosing, following oral delivery of 20 mg/kg and 50 mg/kg of compound 661 (denoted BS661), compared to pregabalin (PGB; 30 mg/kg) and a vehicle control (N=9), mean±SEM, ****p<0.0001 vs. vehicle using Student's T-test) (FIG. 16D); a bar graph showing the latency to fall (seconds) over 6 hours post-dosing following oral delivery of 10 mg/kg, 30 mg/kg, 100 mg/kg and 300 mg/kg of compound 661 (denoted BS661) in male (FIG. 16E) and female (FIG. 16F) rats (N=10, mean±SEM); comparative plots showing the change in colonic temperature compared to Vehicle (° C.) over 12 hours post-dosing, following oral delivery of 20 mg/kg and 50 mg/kg of compound 661 (denoted BS661, compared to ABT102 (ABT; 10 mg/kg) (mean±SEM, N=9, *p<0.05, **p<0.01 vs. relevant Vehicle using student's T-test) (FIG. 16G); comparative plots showing the colonic temperature (° C.) measurements over 24 hours post-dosing following oral delivery of 10 mg/kg, 30 mg/kg, 100 mg/kg and 300 mg/kg of compounds 661 (denoted BS661) compared to vehicle, in male (FIG. 16H) and female (FIG. 161) rats N=4, mean±SEM; comparative plots showing the paw withdrawal latency measurements using hot plate (53° C.), following oral delivery of 20 mg/kg and 50 mg/kg of compound 661 (denoted BS661), compared to ABT102 (ABT; 10 mg/kg) presented as change compared to pre-dose (mean±SEM, N=9, *p<0.05, **p<0.01 vs. appropriate vehicle using student's T-test) (FIG. 16J); bar graphs showing the Paw withdrawal thresholds (PWT) assessed by Von-Frey (VF) measurements in the Spared Nerve Injury (SNI) (FIG. 16K), Distal Tibial Injury (DTI) (FIG. 16L), Chronic Post-Ischemia Pain (CPIP) (FIG. 16M), Post-operative Pain Mechanical hyperalgesia (POP-M) (FIG. 16N) and Post-operative Pain Thermal hyperalgesia (POP-T) (FIG. 16O) endpoints, 6 hours post-dosing of compound 661 (100 mg/kg) (light turquoise), compared at pre-dosing (dark turquoise) and baseline (grey) (N=10, mean±SEM, ***p<0.001, ****p<0.0001 vs. pre-dosing using one way ANOVA followed by Dunnett's test); a bar graph showing the efficacy profile in PWT assessed by VF measurements in the monoiodoacetate-induced osteoarthritis (MIA OA) rat model, 6 hours post-dosing of compound 661 (100 mg/kg) (light turquoise), compared to pre-dosing (dark turquoise) and baseline (grey) (N=10, mean±SEM, ****p<0.001 vs. pre-dosing using one way ANOVA followed by Dunnett's test) (FIG. 16P); and comparative plots showing the dynamic weight bearing (DWB) in MIA-OA rats, over 8 hours post-dosing of compound 661 (denoted BS661) (100 mg/kg) (dark turquoise), compared to naproxen (30 mg/kg) (grey), presented as the % change from vehicle (N=10, mean±SEM, *p<0.05 vs. Vehicle using student's T-test) (FIG. 16Q).

FIG. 17 presents the chemical structures of exemplary compounds derived through structural optimization of compound 661 according to some embodiments of the present invention.

FIGS. 18A-P present whole-cell currents electrophysiology measurements in CHO cells co-expressing hKv7.2/3 and hTRPV1 following hKv7.2/3 activation, in the presence of compound 760 at 0.1 nm (FIG. 18A); data obtained for rat DRG neuron firing in response to capsaicin application with exemplary recording using compound 760 at 1.0 nM, showing inhibition of action potential firing (FIG. 18B); a plot showing the quantification of capsaicin-induced TRPV1 activity inhibition in CHO cells co-expressing Kv7.2/3 and TRPV1 CHO cells, by compound 760 (FIG. 18C); a plot showing the quantification of rat DRG neuron's capsaicin-induced action potential inhibition by compound 760, presented as the drug over control average % inhibition dose-response (FIG. 18D); Representative rat DRG neuron current clamp membrane-potential recording (I=0) with capsaicin (CAP, 1 μM, 6 seconds) stimulation before (left) and after (right) application of 0.1 nM of compound 760 (denoted BSEN760) (FIG. 18E); an efficacy profile of compound 760 in the Spared Nerve Injury (SNI) rat model at doses of 3 mg/kg, 10 mg/kg and 30 mg/kg compared to vehicle control and pregabalin (30 mg/kg) positive control (FIG. 18F); an efficacy profile of compound 760 in the osteoarthritic pain MIA model at doses of 3 mg/kg, 10 mg/kg and 30 mg/kg, compared to a naproxen (30 mg/kg, PO) positive control (FIG. 18G); a scatter plot showing the rat PK profile following oral delivery of 10 mg/kg of compound 760, at 1 and 6 hours post dose, shown as plasma fraction unbound (fu) calculation (FIG. 18H); a bar graph showing the tissue distribution of compound 760 in plasma (green), brain (yellow) and DRG (blue) following oral delivery of 10 mg/kg, at 1 and 6 hours post dose, shown as tissue fraction unbound (FIG. 18I); plot showing the effect on hKv7.3/5 activity, measured in cells expressing Kv7.3/5 using fluorescent assay, by compound 760, presented as drug over control-dose response (Mean±SEM) (FIG. 18J); a representative plot showing the effect on hKv7.4 activity, measured in cells expressing Kv7.4 using fluorescent assay, by compound 760, presented as drug over control-dose response (FIG. 18K); a representative plot showing activation of hERG activity, measured in cells expressing hERG using fluorescent assay, by compound 760, presented as drug over control-dose response (Mean±SEM) (FIG. 18L); a representative scatter plot showing rat PK profile of compound 760 during chronic 5-day BID exposure by oral delivery of up to BID 300 mg/kg. (FIG. 18M); comparative plots showing the colonic core body temperature (° C.) in rats treated with compound 760 at doses of 30 mg/kg (green), 100 mg/kg (grey) and 300 mg/kg (black), compared to a vehicle control, over a time period of 8 hours (FIG. 18N); comparative plots showing the Time to first reaction (change from vehicle in seconds) in rats treated with compound 760 at doses of 30 mg/kg, 100 mg/kg and 300 mg/kg, compared to ABT-102 (10 mg/kg) positive control (FIG. 18O); and a bar graph showing the Time spent on rotarod (% from baseline) in rats treated with compound 760 at doses of 30 mg/kg, 100 mg/kg and 300 mg/kg, compared to a vehicle of compound 760 (vehicle BS), pregabalin (PGB; 30 mg/kg), and the pregabalin vehicle (vehicle PGB) (FIG. 18P).

FIG. 19 presents the structures of exemplary acid precursors usable in the syntheses of exemplary compounds, according to some embodiments of the present invention.

FIG. 20 presents the structures of exemplary amine precursors usable in the syntheses of exemplary compounds, according to some embodiments of the present invention.

FIG. 21 presents an exemplary synthetic protocol for compound 627, according to some embodiments of the present invention.

FIG. 22 presents an exemplary synthetic protocol for compound 421, according to some embodiments of the present invention.

FIG. 23 presents an exemplary synthetic protocol for an acid precursor used in the synthetic protocol of Chemotype 3 compounds, according to some embodiments of the present invention.

FIGS. 24A-B present an exemplary synthetic protocol for an acid precursor used in the synthetic protocol of Chemotype 1 compounds featuring a pyridine B ring, according to some embodiments of the present invention (FIG. 24A) and the chemical structures of exemplary such Chemotype 1 compounds prepared using this acid precursor (FIG. 24B).

FIG. 25 presents an exemplary general synthetic protocol for the preparation of exemplary Chemotype 1 compounds featuring a di-nitrogen heteroaryl B ring according to some embodiments of the present invention. This protocol was used to prepare exemplary compounds 762, 763, 770, 770_3, 843 and 844.

FIG. 26 presents an additional exemplary general synthetic protocol for the preparation of the exemplary Chemotype 1 compounds featuring a di-nitrogen heteroaryl B ring, according to some embodiments of the present invention. This exemplary protocol was used to prepare exemplary compounds 740, 762, 750, 756, 757, 760, 767, 766, 810, 820, 821, 822, 830, 832, 840, 842, 850 and 851.

FIG. 27 presents an exemplary synthetic protocol for the preparation of the exemplary compounds 760 and 767.

### DESCRIPTION OF SPECIFIC EMBODIMENTS OF THE INVENTION

The present invention, in some embodiments thereof, relates to therapeutically active compounds and, more particularly, but not exclusively, to newly designed compounds that are derived from diphenylamine analogs, which feature a dual activity as modulators of both Kv7.2/3 and TRPV1 channels, and which are usable in the treatment of medical conditions that are related to these channels, including, but not limited to, medical conditions associated with neuronal hyper-excitability such as pain, tinnitus and pruritus.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As described in the Examples section that follows, the present inventors have conducted extensive studies aiming at uncovering structural modifications of diphenylamine derivatives that would exhibit improved therapeutic efficacy as dual modulators of both potassium ion (specifically Kv7.2/3) and TRPV1 channels, along with improved pharmacological profile in terms of toxicity, bioavailability, solubility and other pharmacological parameters and/or properties, compared to previously described diphenylamine derivatives.

In the course of these extensive studies, the present inventors have uncovered that certain structural modifications to the structures taught in WO 2009/071947 and WO 2019/073471 lead to compounds that feature an improved performance.

Small molecules usable in the treatment of medical conditions in which dual modulation of the activity of a potassium channel and TRPV1 channel is desirable, have been designed and practiced. These small molecules are particularly usable in the treatment of neuropathic pain; and are usable also in the treatment of other medical conditions in which modulating, as described herein, an activity of one or both of potassium ion (specifically Kv7.2/3) and TRPV1 channels is beneficial.

These compounds were designed according to an approach of targeting multiple hyperexcitability-related mechanisms using a single compound, to achieve greater efficacy and safety. This approach is based on the targeting of two cation channels, a ligand-gated calcium channel and a voltage-gated potassium channel, TRPV1 and Kv7.2/3, respectively, which are co-localized on sensory neurons and are recognized as prominent regulators of neuronal excitability.

The small molecules were designed upon testing the effect of variable modifications, at multiple positions, of previously uncovered small molecules, as described in Example 1. Classical and computational medicinal chemistry together with on target and off target screening methodologies were used in order to identify potent, safe and metabolically stable compounds for in-vivo testing. In addition, species selection studies were conducted so as to assist in finding the appropriate animal model.

Using classical and computational medicinal chemistry, rational structure activity relationship (SAR) studies yielded the design of a few hundred compounds, which were screened for dual targeting properties. The results from these studies provided substantial understanding of the SAR relationship. The main goal was to replace the bridging aniline moiety that is present in compounds such as described in WO 2009/071947 and WO 2019/073471, as it was found to exhibit a significant toxicological alert, leading to the identification of four new chemotypes with potencies in the low μM range and even lower in a heterologous system of ion-channel expression in CHO cells.

To predict a satisfying safety profile of the uncovered compounds, off target activity and cellular toxicity were assayed. Potent compounds on both targets were tested for Kv7.3/5, Kv7.4 and hERG potential liability, potential liver toxicity, TRPV1 polymodality separation and specificity compared to TRPA1. The uncovered compounds exhibit improved tolerability profile, which is attributed mainly to the complete loss of hERG inhibition up to the maximal solubility of the compounds. In addition, some of the uncovered compounds show inhibition of capsaicin-induced activation of TRPV1, while not affecting either pH-induced or heat-induced activation of TRPV1. The selective inhibition of Capsaicin-gated TRPV1 currents, separated from additional inhibition of other TRPV1 activating modalities, is supportive of lack of hyperthermia liability. This TRPV1 polymodality separation was shown to be a good predictor of lack of thermal dysregulation in preclinical and clinical settings.

Identified compounds were progressed to in vitro DMPK studies to assess their potential druggability properties. Both Log D and human liver microsomes (HLM) were evaluated. Generally, the lower Log D values the compounds exhibit (i.e., more polar compounds), a lower HLM value is expected. Indeed, for most compounds with Log D<4, an HLM value<100 (L/min/mg) could be obtained. Good correlation was observed between acceptable HLM and rat liver microsomes (RLM), suggesting that similar metabolic pathways are involved in both species.

Using species-specific constructs and species-specific cells showed that no significant difference can be identified while comparing the inhibition-potency of the tested compounds either from human, rat or pig origin. The lack of a species differences in the inhibitory potency on TRPV1 suggests that rodent model could be used as predictor for efficacy of the uncovered compounds.

To study the inhibitory potency of the identified lead compounds on neuronal excitability in an experimental system of high physiological relevance, primary neonatal rat DRG neurons that comprise of the normal composition of neurons and glia were used. The inhibitory potency of the uncovered compounds was tested both on current-induced or capsaicin-induced neuronal activity.

It was shown that in current stimulation, the inhibitory potency of the tested compounds is mediated and identified through their effects on the voltage-activated effectors, such as the Kv7.2/3 target. In contrast, capsaicin-evoked responses identify the contribution of both the capsaicin-gated TRPV1 target, which depolarizes the membrane upon activation, and consequently activates the voltage-gated Kv7.2/3 target downstream.

Since neuronal excitability is a physiological predictive readout of, inter alia, pain propagation, the add-on effect of dual targeting both TRPV1 and Kv7.2/3 in a pain related system could be examined. It was found, for example, that a representative compound 421-6, with TRPV1 and Kv7.2/3 dual targeting properties (Kv7.2/3 EC50=2.1 μM; TRPV1 IC50=1.8 μM) at 2 μM, dramatically reduced the AP response to both current and capsaicin activation. Compound 421-6 displayed a <100 nM inhibition of capsaicin-induced neuronal firing. This high inhibition potency, significantly above the sum of inhibitions contributed by each target alone, indicates the synergistic effect of the exemplary compound 421-6. This synergism evolves the high inhibition potency of a respective compound and a superior specificity that emerges from a higher activity, which occurs only where both targets are co-expressed and their signaling pathways are crossing, the latter being unique to the nociceptive sensory neurons.

To provide additional physiological support in a human relevant system, human neural progenitor cells (hNPCs) that were differentiated to human sensory neurons were employed. Using spontaneous and induced current application methodologies it was demonstrated, similarly to the significant effect seen in rat DRGs, that the tested compounds have strong inhibition potency on the excitability of human sensory neurons. The superiority of the potency of the newly designed compounds compared to the standard of care drug used in clinics for neuropathic pain treatment, Gabapentin, was also demonstrated.

One of the more promising identified chemotype was further evaluated for its oral bioavailability and metabolic stability. Structural modifications that provide improved potency, solubility and pharmacokinetic performance were identified and accordingly, compounds that show high picomolar potency in electrophysiology recordings, a free plasma concentration above the in-vitro officious dose under the DMPK properties of the compound, translated in vivo to an extended potent pain-relief activity upon oral administration, were practiced. Selected compounds were further tested for their metabolic stability, by determining CYP3A4—Midazolam TDI profile, followed by confirmation that the glutathione conjugation metabolites are not formed in human hepatocytes Met-ID, and it has been uncovered that in order to diminish and preferably abolish a formation of a reactive metabolite, additional modifications are required. Newly designed compounds were prepared and tested and were found to exhibit improved metabolic stability, kinetic solubility, microsomal stability, permeability, plasma protein binding, and thermodynamic stability in intestinal fluids.

Embodiments of the present invention therefore relate to a novel family of compounds derived from diphenylamine derivatives, which exhibit dual activity of opening a potassium channel (e.g., Kv7.2/3) and inhibiting TRPV1 activity, and which exhibit improved pharmacological profile, compared to previously disclosed diphenylamine derivatives; and to uses thereof in the treatment of medical conditions associated with these channels, particularly pain such as neuropathic pain.

### Compounds:

Newly designed compounds according to embodiments of the present invention can be collectively represented by Formula I:

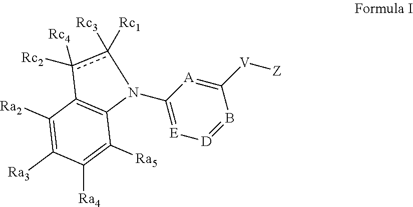

wherein:
A is C—Rb1 or N (nitrogen atom);
B is C—Rb2 or N (nitrogen atom);
D is C—Rb3 or N (nitrogen atom);
E is C—Rb4 or N (nitrogen atom);
Ra2-Ra5 are each independently hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of Ra2-Ra5 form together an alicyclic or aromatic ring;
Rb1, Rb2 and Rb4 are each independently hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Rb substituents form together an alicyclic, heteroalicyclic, aromatic or heteroaromatic ring;
Rc1-Rc4 are each independently absent, hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Rc substituents form together an alicyclic or heterocyclic ring,
The dashed line represents a single bond (when absent) or a double bond (when present), such that when the dashed line represents a double bond, Rc3 and Rc4 are absent;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof, and
Z is represented by Formula II:

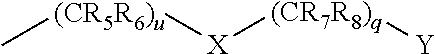

wherein:
u and q are each independently an integer of from 0 to 4, provided that u+q is at least 2;
X is selected from —O— and —NR₉—, or is absent;
Y is a polar hydrophilic group such as OR₁₁, SR₁₁, amine (NR₁₂R₁₃), or amide (—NR₁₂—C(═O)—R₁₄;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring; and
R₉, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring.

According to the present embodiments, at least one, preferably at least two, of A, B, D and E, is N.

The compounds of Formula I therefore comprise a nitrogen-containing heteroaryl (also referred to herein as “Ring B”), coupled to an indole or indoline moiety, and substituted by the -V-Z— moiety at the ortho position with respect to the carbon atom of the heteroaryl that is coupled to the indole.

The compounds of Formula I according to the present embodiments can be considered as modified compounds derived from diphenylamine analogs, in which a phenylamine moiety is replaced by an indole or indoline moiety and another phenyl moiety is replaced by a nitrogen-containing heteroaryl.

According to some of any of the embodiments, the heteroaryl comprises two or three nitrogen atoms.

According to some of any of the embodiments described herein, the heteroaryl is a pyrimidine, in which D and E are each N, B is C—Rb2 and A is C—Rb1; or in which D and B are each N, A is C—Rb1 and E is C—Rb4.

According to some of any of the embodiments described herein, the heteroaryl is a pyrazine, in which A and D are each N, B is C—Rb2 and E is C—Rb4.

According to some of any of the embodiments described herein, the heteroaryl is a pyridazine, in which A and E are each N, B is C—Rb2 and D is C—Rb3; or in which A and B are each N, D is C—Rb3 and E is C—Rb4; or in which E and B are each N, A is C—Rb1 and D is C—Rb3.

According to some of any of the embodiments described herein, the heteroaryl is a triazine, in which A, D and E are each N, and B is C—Rb2; or in which A, D and B are each N, E is C—Rb4; or in which E, D and B are each N and A is C—Rb1.

According to some of any of the embodiments described herein, at least one, and preferably each, of A and B is N. When both A and B are N, the heteroaryl is a pyrimidine.

According to some of any of the embodiments described herein, at least one, and preferably each, of E and B is N. When both E and B are N, the heteroaryl is a pyrimidine.

According to some of any of the embodiments described herein, at least one of A and E is N, such that at least one of the positions of the heteroaryl that is adjacent to the coupling point to the indole is N.

According to some of any of the embodiments described herein, the heteroaryl is a pyrimidine, and at least one of A and E is N.

According to some of any of the embodiments described herein, Rb1, Rb2 and Rb4, when present, are each hydrogen.

According to some of any of the embodiments described herein, the moiety that is coupled to the heteroaryl, which is also referred to herein as “Ring A”, is an indole, where the dashed line in Formula I represents a double bond, or an indoline, where the dashed line in Formula I is absent, and the respective bond is a single bond.

According to some of any of the embodiments described herein, the moiety that is coupled to the heteroaryl, which is also referred to herein as “Ring A”, is an indole, where the dashed line in Formula I represents a double bond.

According to some of any of the embodiments described herein, when the dashed line is absent, each of Rc1-Rc4 is independently selected from hydrogen, alkyl, haloalkyl, and halo.

According to some of any of the embodiments described herein, when the dashed line represents a double bond, and both Rc3 and Rc4 are absent, Rc1 and Rc2 are each independently selected from hydrogen, alkyl, haloalkyl, and halo.

According to some of any of the embodiments described herein, Rc1 is hydrogen.

According to some of any of the embodiments described herein, at least one of Rc1 and Rc2 is other than hydrogen.

According to some of any of the embodiments described herein, when the dashed line represents a double bond, the stereoconfiguration of the double bond can be cis (Z) or trans (E), when relevant.

According to some of any of the embodiments described herein, Rc2 is selected from halo and haloalkyl and is preferably halo. In some of these embodiments, the halo is chloro. In some of the embodiments, the haloalkyl is a trihalomethyl, for example, a trifluoromethyl. In some embodiments, the haloalkyl is a fluoroalkyl, for example, a trifluoromethyl, trofluoroethyl, pentafluoethyl, etc.

According to some of any of the embodiments described herein, the dashed line represents a double bond, Rc1 is hydrogen Rc2 is other than hydrogen and can be, for example, halo, alkyl or haloalkyl, as described herein.

According to some of any of the embodiments described herein, the dashed line represents a double bond, Rc1 is hydrogen Rc2 is halo, preferably chloro.

According to some of any of the embodiments described herein, the dashed line represents a double bond, Rc1 is hydrogen and Rc2 is a haloalkyl as described herein, preferably a trifluoromethyl.

According to some of any of the embodiments described herein, at least two of the Ra substituents are selected from halo and alkoxy.

According to some of any of the embodiments described herein, at least two of the Ra substituents are each halo, which can be the same or different.

According to some of any of the embodiments described herein, Ra3 and Ra5 are each independently a halo, which can be the same or different.

According to some of any of the embodiments described herein, Ra3 and Ra5 are each chloro.

According to some of any of the embodiments described herein, the dashed line represents a double bond, Rc1 is hydrogen, Rc2 is a haloalkyl as described herein, preferably a trifluoromethyl, or a halo, preferably chloro, and Ra3 and Ra5 are each independently a halo, preferably each is chloro.

According to some of any of the embodiments described herein, Ra2 and Ra4 are each independently hydrogen or alkyl, and preferably are each hydrogen.

According to some of any of the embodiments described herein, k is 1. Optionally, k is 0.

According to some of any of the embodiments described herein, when k is other than 0, R₂ and R₃ are each hydrogen. Alternatively, one or both of R₂ and R₃ is an alkyl, or a haloalkyl, preferably a lower alkyl or a lower haloalkyl (e.g. of 1 to 4 carbon atoms in length).

According to some of any of the embodiments described herein, k is 1 and R₂ and R₃ are each hydrogen.

According to some of any of the embodiments described herein, X is absent, such that Z is an alkylene chain or is an alicyclic ring (a cycloalkyl) substituted by Y.

According to some of any of the embodiments described herein, X is O, such that Z is an alkylene glycol chain terminated with Y, or is an alkylene chain (CR₅R₆)u, linked to a heteroalicyclic ring formed between X and Y, or linked to an alicyclic ring formed between two of R₇, R₈ (e.g., in case q is more than 1).

According to some of any of the embodiments described herein, X is O and Z is an alkylene glycol chain terminated by Y. In some of these embodiments, u is 1, 2, or 3, preferably 2, and q is 1, 2, or 3, preferably 2.

According to some of any of the embodiments described herein, X is absent, and Z is an alkylene chain composed of (CR₅R₆)u and (CR₇R₈)q.

According to some of any of the embodiments described herein, X is absent and Z is an alicyclic ring (a cycloalkyl), formed of (CR₅R₆)u and (CR₇R₈)q where two or more of R₅, R₆, R₇ and R₈ form the ring, and the ring is substituted by Y.

According to some of any of the embodiments described herein, the sum of u and q is at least 2, and in some embodiments it is at least 3, for example, is 3, 4, 5 or 6 or more.

According to some of any of the embodiments described herein, at least one of R₅, R₆, R₇ and R₈ is independently selected from alkyl, haloalkyl and halo, and/or at least two of R₅, R₆, R₇ and R₈ form together an alicyclic ring.

According to some of any of the embodiments described herein, at least two of R₅, R₆, R₇ and R₈ are independently selected from alkyl, haloalkyl and halo.

According to some of any of the embodiments described herein, at least two of R₅, R₆, R₇ and R₈ are each independently an alkyl, preferably a lower alkyl (e.g. of 1 to 4 carbon atoms in length).

According to some of any of the embodiments described herein, q is 1 and at least one or each of R₇ and R₈ is alkyl, preferably a lower alkyl (e.g. of 1 to 4 carbon atoms in length).

According to some of any of the embodiments described herein, u is 1 or 2.

According to some of any of the embodiments described herein, R₅ and R₆ are each hydrogen.

Whenever u is other than 1, it is composed of two or more (CR₅R₆) groups, and R₅ and R₆ is each of these groups can be the same or different.

Whenever q is other than 1, it is composed of two or more (CR₇R₈) groups, and R₇ and R₈ is each of these groups can be the same or different.

According to some of any of the embodiments described herein, q is 1 and u is 1 or 2.

According to some of these embodiments, at least one, and preferably both, of R₇ and R₈ is other than hydrogen, and is preferably an alkyl (e.g., a lower alkyl such as methyl). Alternatively, or in addition, at least one, and preferably both, of R₅ and R₆ in one of the (CR₅R₆) group(s) is other than hydrogen, and is preferably an alkyl (e.g., a lower alkyl such as methyl).

According to some of any of the embodiments described herein, q is 1 and u is 2.

According to some of any of the embodiments described herein, q is 2 and u is 2.

According to some of any of the embodiments described herein, in at least one of the (CR₇R₈) groups, at least one, and preferably both, of R₇ and R₈ is other than hydrogen, and is preferably an alkyl (e.g., a lower alkyl such as methyl). Alternatively, or in addition, at least one, and preferably both, of R₅ and R₆ in one of the (CR₅R₆) group(s) is other than hydrogen, and is preferably an alkyl (e.g., a lower alkyl such as methyl).

According to some of any of the embodiments described herein, X is absent and at least two of R₅, R₆, R₇ and R₈ form together an alicyclic ring. In some of these embodiments, u is 1 and q is 1 and R₅, R₆, R₇ and R₈ form together the alicyclic ring.

In some of any of the embodiments described herein, whenever Z comprises an alicylic ring, the ring is a 3-6-membered ring, or is a 4-membered ring or a 5-membered ring or a 6-membered ring, or is a 5-membered ring or a 6-membered ring.

According to some of any of the embodiments described herein, X is absent and at least two of R₅, R₆, R₇ and R₈ form together an alicyclic ring (a cycloalkyl), such as described herein.

In some of these embodiments, u and q are each 1 and in some embodiments, q is 1 and u is 1 or 2. In some of these embodiments R₇ and R₈ form together an alicyclic ring (a cycloalkyl), and in some other embodiments, all of R₅, R₆, R₇ and R₈ form together an alicyclic ring. The alicyclic ring can be of 3, 4, 5, 6 or more carbon atoms, and in some embodiments, it is a 4-membered ring or a 5-membered ring or a 6-membered ring, or is a 5-membered ring or a 6-membered ring.

According to some of any of the embodiments described herein, X is O, and one or more of R₅, R₆, R₇ and R₈, preferably R₇ and/or R₈ is other than hydrogen (e.g., an alkyl, cycloalkyl, aryl).

According to some of any of the embodiments described herein, X is O, u is 2 or more and two of the R₅ and R₆ form together an alicyclic ring.

According to some of any of the embodiments described herein, X is O, q is 2 or more and two of the R₇ and R₈ form together an alicyclic ring.

According to some of any of the embodiments described herein, X is O, q is 1 or more, u is 1 or more and (CR₇R₈)u and (CR₅R₆)q form together an oxygen-containing heteroalicyclic ring, which is substituted by Y. In some of these embodiments, k is 0. In some of these embodiments, u and q are each 1 and the heteroalicyclic ring is a tetrahydrofuran.

According to some of any of the embodiments described herein, k is 0 or 1, as described herein, and Z is an alicyclic or heteroalicyclic ring, substituted by Y, as described herein in any of the respective embodiments. In exemplary embodiments, Z is an oxygen-containing heteroalicyclic ring, which can be 3-membered, 4-membered, 5-membered or 6-membered oxygen-containing alicyclic ring. In some of these embodiments, the heteroalicyclic ring is a tetrahydrofuran, substituted by Y. In some of any of these embodiments, Y is hydroxy.

According to some of any of the embodiments described herein, X is NR₉, and R₉ forms together with one or more of R₅, R₆, R₇ and R₈ an heteroalicyclic ring (e.g., a 4-membered ring or a 5-membered ring or a 6-membered ring, or is a 5-membered ring or a 6-membered heteroalicyclic ring).

In some of any of the embodiments described herein, at least one of R₅, R₆, R₇ and R₈ is independently selected from alkyl, haloalkyl and halo, and/or at least two of R₅, R₆, R₇ and R₈ form together an alicyclic ring, as described herein, and at least one or at least two of the Ra substituent(s) is halo, as described herein. In some of any of these embodiments, m is 1 and the Rb substituent is halo, e.g., fluoro, as described herein in any of the respective embodiments.

In some of any of the embodiments described herein, at least one of R₅, R₆, R₇ and R₈ is independently an alkyl and/or at least two of R₅, R₆, R₇ and R₈ form together an alicyclic ring, as described herein, and at least one or at least two of the Ra substituent(s) is halo, as described herein.

In some of any of these embodiments, m is 1 and the Rb substituent is halo, e.g., fluoro, as described herein in any of the respective embodiments.

In some of any of the embodiments described herein, at least one of R₅, R₆, R₇ and R₈ is independently selected from alkyl, haloalkyl and halo, and/or at least two of R₅, R₆, R₇ and R₈ form together an alicyclic ring, as described herein, and at least one or at least two of the Ra substituent(s) is halo, as described herein. In some of any of these embodiments, m is 1 and the Rb substituent is halo, e.g., fluoro, as described herein in any of the respective embodiments.

In some of any of the embodiments described herein, at least one of R₅, R₆, R₇ and R₈ is independently an alkyl, and/or at least two of R₅, R₆, R₇ and R₈ form together an alicyclic ring, as described herein, and at least one or at least two of the Ra substituent(s) is halo, as described herein.

In some of any of these embodiments, m is 1 and the Rb substituent is halo, e.g., fluoro, as described herein in any of the respective embodiments.

According to some of any of the embodiments described herein, Y is hydroxy, such that Y is OR₁₀ and R₁₀ is hydrogen.

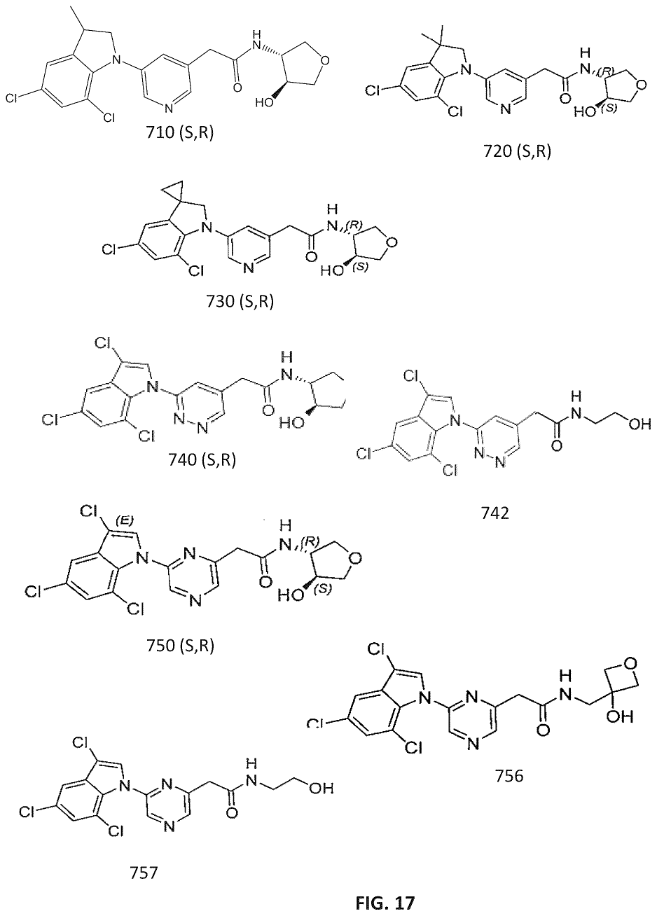

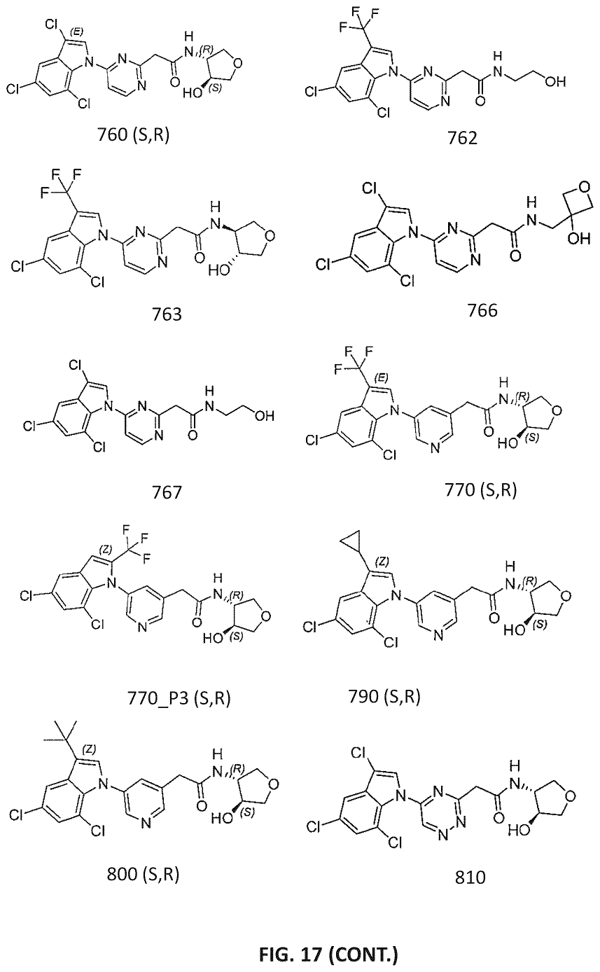

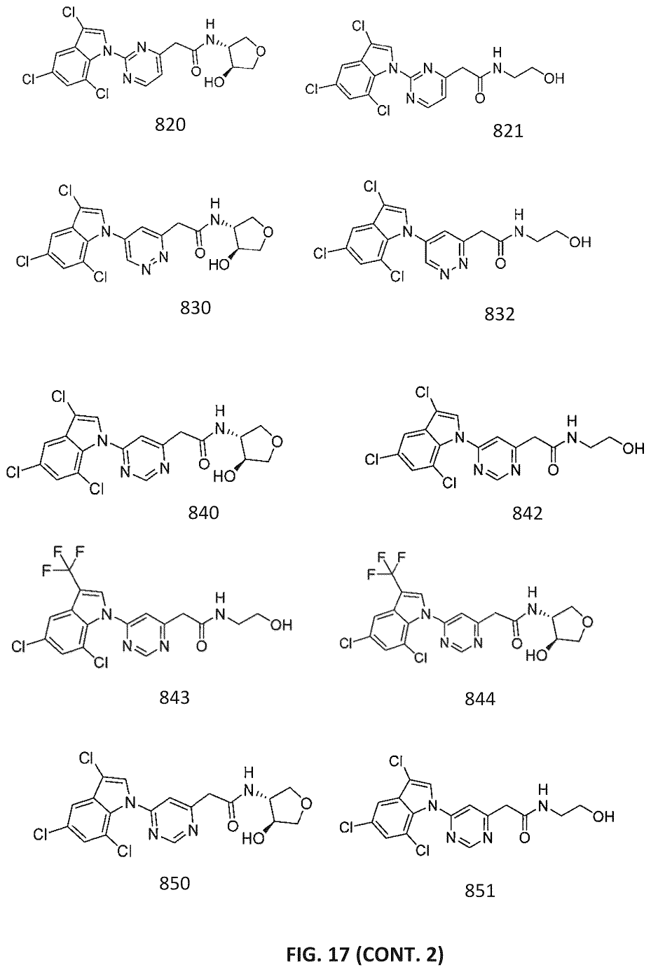

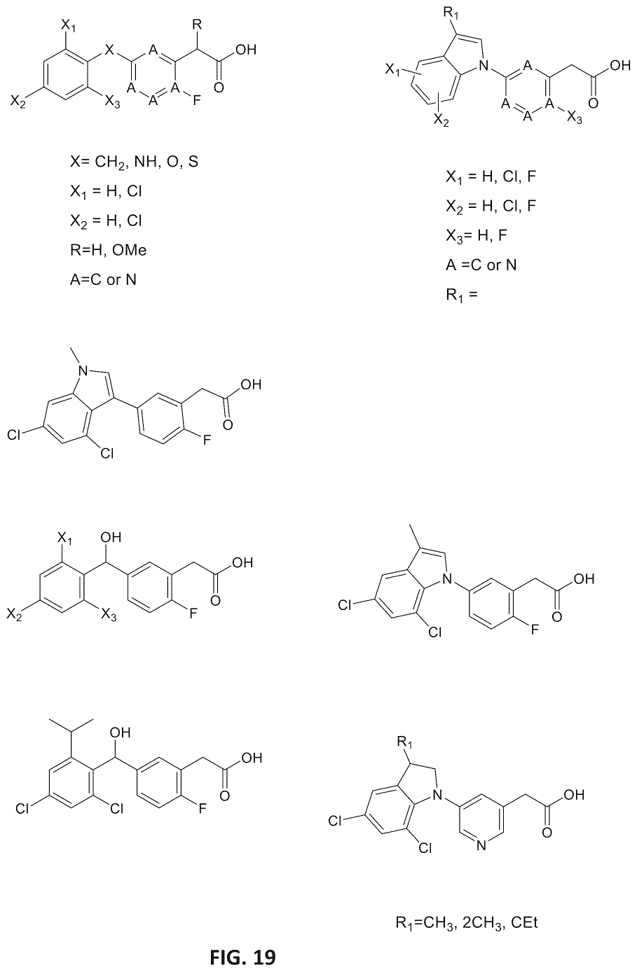

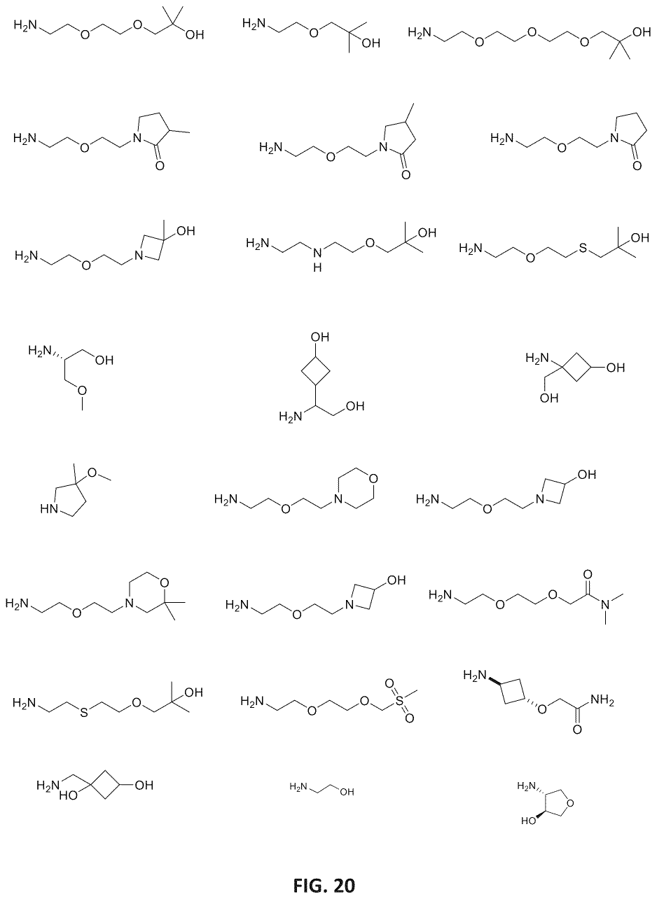

Exemplary compounds according to the present embodiments are presented in FIG. 17. Additional compounds are formed by reacting one of the acid precursors presented in FIG. 19 with one of the amine precursors as presented in FIG. 20. Any combination of an acid precursor and an amine precursor is contemplated.

Exemplary compounds according to the present embodiments include compounds 760, 767 and 843, as described herein.

The compounds of the present embodiments can be readily prepared by methods known in the art, typically by coupling an acid precursor to an amine precursor, as described herein.

Exemplary synthetic pathways are described in the Examples section that follows.

According to some of any of the embodiments described herein, the newly designed compounds are such that exhibit one or more, or two or more, or three or more, and preferably all of the following characteristics:
Log D, determined as described herein, lower than 4;
Ligand-lipophilicity efficiency (LLE), determined as described herein, higher than 3, or higher than 5;
HLM Clint, determined as described herein, lower than 100 or lower than 60 ml/min/kg;
Kinetic solubility higher than 20 or higher than 30 or higher than 40 micromolar;
Caco-2 efflux ratio, determined as described herein, lower than 3; and
A time-dependent inhibition (TDI) of CYP3A4 enzyme in the presence of midazolam, determined by the (−)NADPH/(+)NADPH ratio as described herein, lower than 1.57.

For any of the embodiments described herein, and any combination thereof, the compound may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., amine and/or amide and/or a nitrogen atom in a heterocyclic group) group of the compound which is in a positively charged form (e.g., wherein the basic group is protonated), in combination with at least one counter-ion, derived from the selected acid, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment, which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter, they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, provide improved bioavailability by oral administration over the parent drug. A prodrug may also have improved solubility as compared with the parent drug in pharmaceutical compositions. Prodrugs are also often used to achieve a sustained release of the active compound in vivo. Exemplary prodrugs of compounds according to some of the present embodiments include esters of a hydroxy group (e.g., a hydroxy group present as a substituent or at the terminus of the side chain Z in Formula I), including carboxylic esters, phosphate esters, and the like.

An exemplary prodrug according to some embodiments of the present invention is a phosphate ester prodrug as exemplified below, which is converted to the respective alcohol. According to some of these embodiments, Y is OR₁₁ and R₁₁ is such that forms a phosphate.

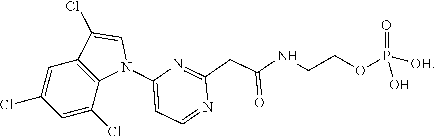 [SMILES: O=C(Cc1nccc(-n2cc(Cl)c3cc(Cl)cc(Cl)c32)n1)NCCOP(=O)(O)O]

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

### Pharmaceutical Composition:

In any of the methods and uses described herein, the compounds of the present embodiments can be utilized per se, or in a pharmaceutical composition where it is mixed with suitable carriers or excipients.

According to an aspect of some embodiments of the present invention there is provided a pharmaceutical composition comprising the compound as described herein and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound or combination of compounds which are accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

According to some embodiments of the present invention, a compound or pharmaceutical composition as described herein are administered topically.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

According to some embodiments of the present invention, the compound or pharmaceutical composition as described herein are administered so as to deliver the compound to the central and/or peripheral nervous system.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Local administration by perineural injection, or by means of eye or ear drops, is also contemplated for delivering the active compounds to the central nervous system.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of the active ingredient (a compound as described herein) effective to prevent, alleviate or ameliorate symptoms of a medical condition as described herein or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue (e.g., plasma) levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine concentrations in the relevant tissue (e.g., plasma and/or brain).

In some embodiments of any of the embodiments described herein, an effective amount of the compound is less than 100 μM. In some embodiments, an effective amount is less than 10 μM. In some embodiments, an effective amount is less than 5 μM. In some embodiments, an effective amount is less than 1 μM. In some embodiments, an effective amount is less than 0.5 μM. In some embodiments, an effective amount is less than 0.1 μM.

In some embodiments of any of the embodiments described herein, an effective amount of the compound ranges from 1 pM to 1 mM, or from 1 pM to 100 μM, or from 100 pM to 100 μM, or from 100 pM to 10 μM, or from 100 pM to 1 μM, or from 100 pM to 500 nM, or from 100 pM to 100 nM, including any intermediate values and subranges therebetween.

In some embodiments of any of the embodiments described herein, an effective amount is at least 100% of the IC50 of the compound towards TRPV1 and/or Kv7.2/3. In some embodiments, an effective amount is at least 200% of the IC50 of the compound towards TRPV1 and/or Kv7.2/3. In some embodiments, an effective amount is at least 300% of the IC50 of the compound towards TRPV1 and/or Kv7.2/3. In some embodiments, an effective amount is at least 500% of the IC50 of the compound towards TRPV1 and/or Kv7.2/3. In some embodiments, an effective amount is at least 1000% of the IC50 of the compound towards TRPV1 and/or Kv7.2/3.

In some embodiments of any of the embodiments described herein, an effective amount of a compound as described herein is at the nM range (e.g., from 0.001 to 1,000 nM, or from 0.001 nM to 100 nM).

In some embodiments of any of the embodiments described herein, an effective amount of a compound as described herein is lower by at least 10%, or by at least 20, 30, 40, 50, 60, 70, 80, 90, 100%, or even more, than an amount that causes hERG inhibition.

In some embodiments of the present invention, the amount of the compound or pharmaceutical composition to be administered required to achieve a therapeutic effect (e.g., a dosage or a therapeutically effective amount of the compound as described herein) is lower than an amount of previously described compounds known to exhibit the same therapeutic effect by at least 20%, or at least 30%.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

As used herein, the term “subject” includes mammals, preferably human beings at any age which suffer from the pathology. Preferably, this term encompasses individuals who are at risk to develop the pathology.

It will be appreciated that the compounds or pharmaceutical compositions as described herein can be provided alone or in combination with other active ingredients, which are well known in the art for alleviating the medical condition (e.g., neuropathic pain) and/or for activating a potassium channel as described herein and/or for inhibiting an activity of TRPV1.

In some embodiments, the compounds or pharmaceutical compositions as described herein may be administered with an activator of a potassium channel as described herein (e.g., Kv7.2/3), either together in a co-formulation or in separate formulations.

The pharmaceutical composition may further comprise additional pharmaceutically active or inactive agents such as, but not limited to, an anti-bacterial agent, an antioxidant, a buffering agent, a bulking agent, a surfactant, an anti-inflammatory agent, an anti-viral agent, a chemotherapeutic agent and an anti-histamine, and/or an additional agent usable in treating a medical condition, disease or disorder as described herein.

### Uses:

According to an aspect of some embodiments of the present invention there is provided a compound as described herein or a pharmaceutical composition as described herein, for use in modulating an activity of a voltage-dependent potassium channel.

According to an aspect of some embodiments of the present invention there is provided a method of modulating an activity of a voltage-dependent potassium channel, which comprises contacting the potassium channel with a compound or a pharmaceutical composition as described herein. The contacting can be effected in vitro, e.g., by contacting a cell, a tissue or an organ which express the channel with the compound or composition, or in vivo, by administering to a subject in need thereof a therapeutically effective amount of the compound or composition.

In some embodiments, the potassium channel is Kv7.2/7.3 (which is also referred to herein interchangeably as Kv7.2/3).

In some embodiments, the modulating comprises opening the potassium channel.

According to an aspect of some embodiments of the present invention there is provided a compound as described herein or a pharmaceutical composition as described herein, for use in modulating an activity of a TRPV1 channel.

According to an aspect of some embodiments of the present invention there is provided a method of modulating an activity of a TRPV1 channel, which comprises contacting the TRPV1 channel with a compound or a pharmaceutical composition as described herein. The contacting can be effected in vitro, e.g., by contacting a cell, a tissue or an organ which express the channel with the compound or composition, or in vivo, by administering to a subject in need thereof a therapeutically effective amount of the compound or composition.

In some embodiments, the modulating comprises inhibiting the activity of the TRPV1 channel (e.g., blocking the channel).

According to an aspect of some embodiments of the present invention there is provided a compound as described herein or a pharmaceutical composition as described herein, for use in modulating an activity of both a voltage-dependent potassium channel and a TRPV1 channel, as described herein in any of the respective embodiments.

According to an aspect of some embodiments of the present invention there is provided a method of modulating an activity of a voltage-dependent potassium channel and of a TRPV1 channel, which comprises contacting these channels with a compound or a pharmaceutical composition as described herein. The contacting can be effected in vitro, e.g., by contacting a cell, a tissue or an organ which express these channels with the compound or composition, or in vivo, by administering to a subject in need thereof a therapeutically effective amount of the compound or composition.

According to an aspect of some embodiments of the present invention there is provided a compound as described herein or a pharmaceutical composition as described herein, for use in treating a medical condition associated with an activity of a voltage-dependent potassium channel and/or of a TRPV1 channel.

According to an aspect of some embodiments of the present invention there is provided a compound as described herein or a pharmaceutical composition as described herein, for use in treating a medical condition associated with an activity of a voltage-dependent potassium channel and of a TRPV1 channel.

According to an aspect of some embodiments of the present invention there is provided a method of treating a medical condition associated with an activity of a voltage-dependent potassium channel and/or of a TRPV1 channel in a subject in need thereof, the method comprising administering to the subject a therapeutically effective amount of a compound or a pharmaceutical composition as described herein in any of the respective embodiments and any combination thereof.

According to some of any of the embodiments described herein, the medical condition is such that modulating an activity of one, and preferably both, of a voltage-dependent potassium channel and a TRPV1 channel, as described herein, is beneficial.

According to some of any of the embodiments described herein, the medical condition is such that opening a voltage-dependent potassium channel and inhibiting an activity (e.g., blocking) a TRPV1 channel, as described herein, is beneficial.

Such medical conditions include, for example, conditions associated with impaired electrical activity of cells, particularly of the nervous system; condition associated with excessive neuronal excitation or abnormal excitability in neuronal tissues; and/or conditions associated with pain sensation, in which reducing pain perception is beneficial.

An exemplary medical condition is neuropathic pain.

Any other medical conditions (pathologies, conditions, diseases and/or disorders) that are associated with TRPV1 channel functioning and/or a voltage-dependent potassium channel as described herein are contemplated.

Exemplary medical conditions that are beneficially treatable by the TRPV1 inhibitors (e.g., blockers) described herein (compounds having general Formula I) include, but are not limited to, epilepsy, pain related conditions such as neurogenic pain, neuropathic pain, allodynia, pain associated with inflammation, and pain associated with pancreatitis, bipolar disorder, mood disorder, psychotic disorder, schizophrenia, anxiety, tinnitus and a motor neuron disease, bladder overactivity, urinary incontinence, persistent visceral hypersensitivity, including irritable bowel syndrome (IBD), chronic cough, and cancer (for example, squamous cell carcinoma, prostate carcinoma and pancreatic cancer).

There is much pathology, conditions and disorders that is associated with defective potassium channel functioning. Just as other potassium channel opening compounds, the compounds described herein are for use within the framework of a treatment for pathologies, conditions, disease and disorders associated with defective potassium channel functioning, so as to treat, ameliorate, prevent, inhibit, or limit the effects of the conditions and pathologies in animals including humans.

Exemplary medical conditions that are beneficially treatable by the potassium channel openers described herein include, but are not limited to, central or peripheral nervous system disorders such as ischemic stroke, migraine, ataxia, Parkinson's disease, bipolar disorders, trigeminal neuralgia, spasticity, mood disorders, brain tumors, psychotic disorders, schizophrenia, pruritus, myokymia, neurogenic pain, neuropathic pain, seizures, epilepsy, tinnitus, hearing and vision loss, anxiety and motor neuron diseases. The compounds described herein can further be beneficially used as neuroprotective agents (e.g., to prevent stroke and the like). The compounds described herein are also useful in treating disease states such as gastroesophogeal reflux disorder and gastrointestinal hypomotility disorders.

The compounds disclosed herein can also be used as potent candidates for treating a variety of medical conditions wherein depressing the cortical and/or peripheral neuron activity is beneficial, such as, for example, epilepsy, ischemic stroke, migraine, ataxia, myokymia, neurogenic pain, neuropathic pain, Parkinson's disease, bipolar disorder, trigeminal neuralgia, spasticity, mood disorder, psychotic disorder, schizophrenia, brain tumor, hearing and vision loss, anxiety, tinnitus and a motor neuron disease.

According to an aspect of some embodiments of the present invention, the compound or the composition as described herein is for use in depressing a cortical and/or peripheral neuron activity and/or in treating a condition in which depressing a cortical and/or peripheral neuron activity in a subject is beneficial, as described herein.

The compounds disclosed herein are particularly usable for treating medical conditions associated with neuronal hyperexcitability.

According to an aspect of some embodiments of the present invention, the compound or the composition as described herein is for use in treating a medical condition associated with hyperexcitability (e.g., neuronal hyperexcitability) in a subject in need thereof.

According to an aspect of some embodiments of the present invention, the compound or the composition as described herein is for use in the preparation of a medicament for treating a medical condition associated with hyperexcitability (e.g., neuronal hyperexcitability) in a subject in need thereof.

According to an aspect of some embodiments of the present invention, there is provided a method of treating a medical condition associated with hyperexcitability (e.g., neuronal hyperexcitability) in a subject in need thereof, which is effected by administering to the subject a therapeutically effective amount of a compound or a composition as described herein in any of the respective embodiments and any combination thereof.

Medical conditions associated with neuronal hyperexcitability include, but are not limited to, epilepsy, neurodegeneration, neurodevelopmental disorders, Stroke, retinal degeneration, tinnitus, spinal cord injury, traumatic brain injury, hearing loss (through noise overexposure or ototoxicity), neuropathic pain, attention deficit hyperactivity disorder, autism, central pain syndromes, neurodegenerative diseases, multiple sclerosis, Alzheimer's disease, amyotrophic lateral sclerosis (ALS), Parkinson's disease, frontotemporal dementia, schizophrenia, Rasmussen's encephalitis, Huntington's disease, alcoholism or alcohol withdrawal, over-rapid benzodiazepine withdrawal, neonatal convulsions, episodic ataxia, myokymia, cerebral ischemia, cerebral palsy, asphyxia, anoxia, prolonged cardiac surgery, hypoglycemia, AIDS related dementia and anxiety disorders.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halo, carbonyl, thiocarbonyl, O-carbamyl, N-carbamyl, O-thiocarbamyl, N-thiocarbamyl, C-amido, N-amido, C-carboxy, O-carboxy, nitro, sulfonamido, trihalomethanesulfonamido, silyl, guanyl, guanidino, ureido, amino or NR′R″, wherein R′ and R″ are each independently hydrogen, alkyl, cycloalkyl, aryl, carbonyl, sulfonyl, trihalomethysulfonyl and, combined, a five- or six-member heteroalicyclic ring.

A “haloalkyl” groups describes an alkyl, as defined herein, substituted by one or more halo substituents, as defined herein. In some embodiments, the haloalkyl is an alkyl substituted by two or more, or three of more, halo substituents. In some embodiments, each of the halo substituents is fluoro. In some embodiments, a haloalkyl is —CF₃ or —CF₂H.

A “cycloalkyl” group refers to an all-carbon monocyclic or fused ring (i.e., rings which share an adjacent pair of carbon atoms) group wherein one of more of the rings does not have a completely conjugated pi-electron system (an alicyclic ring). Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halo, carbonyl, thiocarbonyl, C-carboxy, O-carboxy, O-carbamyl, N-carbamyl, C-amido, N-amido, nitro, amino and NR′R″ as defined herein.

An “alkenyl” group refers to an alkyl group, which consists of at least two carbon atoms and at least one carbon-carbon double bond.

An “alkynyl” group refers to an alkyl group, which consists of at least two carbon atoms and at least one carbon-carbon triple bond.

In some embodiments, whenever an alkyl substituent is indicated, it can be replaced by an alkynyl or an alkynyl, as defined herein.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, halo, trihalomethyl, alkyl, hydroxy, alkoxy, aryloxy, thiohydroxy, thiocarbonyl, C-carboxy, O-carboxy, O-carbamyl, N-carbamyl, O-thiocarbamyl, N-thiocarbamyl, C-amido, N-amido, sulfinyl, sulfonyl, amino and NR′R″ as defined herein.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, halo, trihalomethyl, hydroxy, alkoxy, aryloxy, thiohydroxy, thiocarbonyl, sulfonamido, C-carboxy, O-carboxy, sulfinyl, sulfonyl, O-carbamyl, N-carbamyl, O-thiocarbamyl, N-thiocarbamyl, C-amido, N-amido, amino or NR′R″ as defined herein.

Herein throughout, and, for example, in Formulae II, III, IV and V, whenever pyridine is described as a heteroaryl B ring, it is to be noted that other nitrogen-containing heteroaryls are also contemplated.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, cycloalkyl, aryl, heteroaryl, halo, trihalomethyl, hydroxy, alkoxy, aryloxy, thiohydroxy, thioalkoxy, thioaryloxy, cyano, nitro, carbonyl, thiocarbonyl, C-carboxy, O-carboxy, O-carbamyl, N-carbamyl, O-thiocarbamyl, N-thiocarbamyl, sulfinyl, sulfonyl, C-amido, N-amido, amino and NR′R″ as defined above.

As used herein, a “cyclic group” describes an alicyclic group (a cycloalkyl), an aryl, a heteroaryl or an heteroalicyclic.

A “hydroxy” group refers to an —OH group.

An “azido” group refers to a —N═N group.

An “alkoxy” group refers to both an —O-alkyl and an —O-cycloalkyl group, as defined herein.

A “haloalkoxy” group describes an O-alkyl group where the alkyl is a haloalkyl as described herein.

An “aryloxy” group refers to both an —O-aryl and an —O-heteroaryl group, as defined herein.

A “thiohydroxy” or “thiol” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, cycloalkyl, aryl, heteroaryl (bonded through a ring carbon) or heteroalicyclic (bonded through a ring carbon) as defined herein.

An “aldehyde” group refers to a carbonyl group, where R′ is hydrogen.

A “thiocarbonyl” group refers to a —C(═S)—R′ group, where R′ is as defined herein.

The term “carboxylate” encompasses C-carboxylate and O-carboxylate.

A “C-carboxy” group refers to a —C(═O)—O—R′ groups, where R′ is as defined herein.

An “O-carboxy” group refers to an R′C(═O)—O— group, where R′ is as defined herein.

A “carboxylic acid” group refers to a C-carboxyl group in which R′ is hydrogen.

A “halo” group refers to fluorine, chlorine, bromine or iodine.

A “trihalomethyl” group refers to a —CX₃ group wherein X is a halo group as defined herein.

A “trihalomethanesulfonyl” group refers to an X₃CS(═O)₂— group wherein X is a halo group as defined herein.

A “sulfinyl” group refers to an —S(═O)—R′ group, where R′ is as defined herein.

A “sulfonyl” group refers to an —S(═O)₂—R′ group, where R′ is as defined herein.

The term “sulfonylamide” encompasses S-sulfonylamide and N-sulfonylamido.

An “S-sulfonamido” group refers to a —S(═O)₂—NR′R″ group, with R′ is as defined herein and R″ is as defined for R′.

An “N-sulfonamido” group refers to an R'S(═O)₂—NR″ group, where R′ and R″ are as defined herein.

A “trihalomethanesulfonamido” group refers to an X₃CS(═O)₂NR′— group, where R′ and X are as defined herein.

The term “carbamate” encompasses O-carbamyl and N-carbamyl.

An “O-carbamyl” group refers to an —OC(═O)—NR′R″ group, where R′ and R″ are as defined herein.

An “N-carbamyl” group refers to an R′OC(═O)—NR″— group, where R′ and R″ are as defined herein.

The term “thiocarbamate” encompasses O-thiocarbamyl and N-thiocarbamyl.

An “O-thiocarbamyl” group refers to an —OC(═S)—NR′R″ group, where R′ and R″ are as defined herein.

An “N-thiocarbamyl” group refers to an R″OC(═S)NR′— group, where R′ and R″ are as defined herein.

An “amino” group refers to an —NR′R″ group, where R′ and R″ are as defined herein.

An “alkylamino” group refers to an amine group is which one of R′ and R″ is alkyl (monoalkylamine) or in which both R′ and R″ are each independently an alkyl (dialkylamine).

The term “amide” encompasses C-amido and N-amido.

A “C-amido” group refers to a —C(═O)—NR′R″ group, where R′ and R″ are as defined herein.

An “N-amido” group refers to an R′C(═O)—NR″ group, where R′ and R″ are as defined herein.

A “quaternary ammonium” group refers to an —NHR′R″⁺ group, wherein R′ and R″ are independently alkyl, cycloalkyl, aryl or heteroaryl.

An “ureido” group refers to an —NR′C(═O)—NR″R′″ group, where R′ and R″ are as defined herein and R′″ is defined as either R′ or R″.

A “guanidino” group refers to an —R′NC(═N)—NR″R′″ group, where R′, R″ and R′″ are as defined herein.

A “guanyl” group refers to an R′R″NC(═N)— group, where R′ and R″ are as defined herein.

A “nitro” group refers to an —NO₂ group.

A “cyano” group refers to a —C≡N group.

A “silyl” group refers to a —SiR′R″R′″, where R′, R″ and R′″ are as defined herein.

As used herein, the term “alkylene glycol” describes a —O—[(CR′R″)_{z}—O]_{y}—R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo(alkylene glycol).

A “leaving group” as used herein and in the art describes a labile atom, group or chemical moiety that readily undergoes detachment from an organic molecule during a chemical reaction, while the detachment is typically facilitated by the relative stability of the leaving atom, group or moiety thereupon. Typically, any group that is the conjugate base of a strong acid can act as a leaving group. Representative examples of suitable leaving groups according to some of the present embodiments include, without limitation, trichloroacetimidate, acetate, tosylate, triflate, sulfonate, azide, halide (halo, preferably bromo or iodo), hydroxy, thiohydroxy, alkoxy, cyanate, thiocyanate, nitro and cyano.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

### Solubility Assay:

Solubility measurements were performed prior to each in vitro assay to ensure accurate dose response and reproducibility. Briefly, each compound was dissolved to 60 mM in DMSO and calibration curve was performed from 1.5-200 μM using absorbance spectra analysis and determination of λmax. Calibration curve served as the readout for all following assays. The 60 mM stock solution was used to make a 120 μM target concentration of 0.2% DMSO in the appropriate buffer of the in vitro related assay (i.e. HHBS buffer). Solubilized compounds were vortex for 10 minutes followed by centrifugation for 15 minutes in 5000 RPM and the supernatant was recovered for absorbance spectra analysis between 200-400 nm.

### On Target Assays:

Two kits that enable hKv7.2/3 and hTRPV1 read-outs in a high content screen (HCS) manner were used. Electrophysiology was used to validate the readouts for selected compounds directly recording their modulations of ion-channel currents, to study the inhibition potency of our compounds on neuronal excitability, and to study the modulations of TRPV1 pH-gating.

### Kv7.2/7.3:

Cell culture: Chinese hamster ovary (CHO) cell-line cells with a constitutive expression of human Kv7.2/3 channels (B'SYS GmbH, Switzerland) were cultured in F-12 nutrient mixture (Biological Industries) supplemented with 10% fetal bovine serum (Biological Industries) and 1% penicillin-streptomycin (Biological Industries) in a humidified 5% CO₂ incubator at 37° C. To maintain the stability of human Kv7.2/7.3 expression antibiotic selection was added (Puromycin 5 μg/ml).

High content screen (HCS) using FLIPR Potassium Assay Kit: A FLIPR Potassium Assay Kit (R8222 FLIPR Potassium Assay Explorer Kits, Molecular Devices) was used with the CHO/hKv7.2/3 cell line to screen molecules against the hKv7.2/7.3 ion channel target. The assay exploits the permeability of thallium ions (Tl+) through potassium (K+) channels. In this assay, Tl+ indicator dye is used to produce a bright fluorescent signal upon the binding to Tl+ conducted through the potassium channels. The intensity of the Tl+ signal is proportional to the number of potassium channels in the open state. Therefore, it provides a functional indication of the potassium channel activity. To activate the voltage-gate potassium channels, the cells were stimulated with a mixture of K+ and Tl+ to depolarize the cell membrane. The fluorescence increase in the assay represents the influx of Tl+ into the cell specifically through the potassium channel, providing a functional read-out of hKv7.2/3 activity using a fluorescent plate reader coupled with injectors used to activate the channels.

According to optimization process to achieve uniform and consistent screening conditions the following protocol was established: Cells were seeded in 384-well, black-walled, clear-bottomed Greiner #781091, at a density of 5000 cells per well 24 hours before the assay conductance and incubated overnight in their normal growth medium. On the experiment day the medium was replaced with HBSS, HEPES, and the test compound/vehicle (0.2% DMSO). Dye solution (prepared according to the manufacturer) was added to the plates, which were incubated for 1.5 h light-protected in room temperature. Tecan Spark reader plate injectors were primed with Tl+(1 mM Tl₂SO₄) or Tl++K+(1 mM Tl₂SO₄; 5 mM K₂SO₄) for channel activation and data correction. Compounds effects are compared to vehicle control and NH91 or Retigabine positive control. To calculate EC50 values, the data was fitted to sigmoidal regression using Prism GraphPad. The fitting was constrained to a minimum of 1 and a maximal response of about 3.6 unless a different maximal response could be clearly identified.

### Kv7.2/7.3 Electrophysiology:

Extracellular (Bath) solution: The solution was composed of (in mM): NaCl, 140; KCl, 4; CaCl₂, 1.8; MgCl₂, 1.2; Glucose, 11; HEPES, 5.5. pH was adjusted to 7.3 with NaOH. Osmolarity was adjusted to 310 mOsm with Sucrose.

Intracellular (Pipette) solution: Pipettes were pulled from borosilicate glass (Warner Instrument Corp, USA) with a resistance of 3-7 MΩ and were filled with internal solution composed of (in mM): KCl, 130; MgCl₂, 1; K ATP, 5; EGTA, 5 (replaced by BAPTA in rat DRG neurons recordings); HEPES, 10. pH was adjusted to 7.3 with KOH. Osmolarity was adjusted to 290 mOsm with Sucrose.

A programmable valve-linked pressurized perfusion system (AutoMate Scientific) was used for local application of compounds nearby the cell recorded in a consistent flow rate of 2-3 ml/min. Series resistance was corrected and data were sampled at 5 kHz and low pass filtered at 2.4 kHz using MultiClamp 700B amplifier with pCLAMP11 software (Molecular Devices, USA).

Kv7.2/7.3 recordings: To evaluate the effect of test compounds on hKv7.2/3 currents at −40 mV membrane potential (the threshold for action potential initiation), a −40 mV pulse train protocol was conducted: Membrane potential is held on −90 mV and is then clamped to −40 mV for 1.5 seconds, followed by clamping the membrane to −60 mV to obtain tail currents for 0.75 seconds, and back to the −90 mV holding potential. An interval of 30 seconds in −90 mV holding potential is kept between the sweeps.

After a steady baseline current is achieved, test compound is locally applied using the pressurized perfusion system until a maximal and stable channel modulation in −40 mV is achieved, as confirmed by recording of three similar consequent responses. Thereafter, in a similar manner, cells are perfused back to their control bath solution, to assess the reversibility of the effect of the compounds.

The current obtained at −40 mV membrane potential when test compound is injected is divided by the averaged current at −40 mV, recorded before test-compound application and following its washout, to evaluate the drug/control response.

### TRPV1:

Cell culture: Chinese hamster ovary (CHO) cell-line cells with constitutive expression of human hTRPV1 channels (B'SYS GmbH, Switzerland) were cultured in F-12 nutrient mixture (Biological Industries) supplemented with 10% fetal bovine serum (Biological Industries) and 1% penicillin-streptomycin (Biological Industries) in a humidified 5% CO2 incubator at 37° C. To maintain the stable expression of hTRPV1, antibiotic selection (G418 500 μg/ml) was applied.

Species Selection Rat, Pig, and Human DNA constructs and DNA transfection: hTRPV1 cells were transfected with DNA constructs (Genscript). The coding regions of human (NM_080704.4), rat (NM_031982.1), and pig (XM_013981216.2) TRPV1 were inserted similarly into the multiple cloning site of pcDNA3.1(+) between the Hind III and BamH I restriction site. For CHO transfection, 1×10⁶ cells were suspended in a reaction mix (Amaxa™ 4D-Nucleofector™-LONZA) containing 5 μg of DNA for each transfection in a cuvette. The cells were transformed (Amaxa, DT-133 program for CHO transfection with p-3 Kit) and recovered for 10 minutes in RT in the hood and gently transferred with Lonza pipette dropwise into two wells of 6-wells containing prewarm medium (Ab free) to settle down. Following transfection, day before the experiment, the hTRPV1 cells were plated in a black, flat 384-well plate (Greiner #781091). The screening of molecules against CHO cells transiently transfected with rTRPV1 vs hTRPV1 vs pTRPV1 ion channel was performed using the Fluo-8 No Wash Calcium Assay Kit (Abcam, ab112129).

High content screen (HCS) using Fluo-8 No Wash Calcium flux Assay Kit: Fluo-8 No Wash Calcium Assay Kit (Abcam, ab112129) was used with CHO stably or transiently expressing TRPV1 to screen compounds against the hTRPV1 ion channel target. The cells were pre-loaded with Fluo-8AM which is membrane permeable. The AM groups of the Fluo-8AM were then being cleaved by intracellular esterase, trapping the Fluo8 in the cell. Calcium influx through activated TRPV1 channels significantly increases the fluorescence of Fluo-8. The relative fluorescence signal was calculated following background subtraction, comparing the fluorescence at each time point to its maximal level measured following Ionomycin application in the end.

According to optimization process to achieve uniform and consistent screening condition of molecules the following protocol was established: Cells were seeded in 384-well, black-walled, clear-bottomed, at a density of 5,000 cells per well 24 h before the assay conductance and incubated overnight in their normal growth medium. On the experiment day the medium was replaced in each well with HBSS, HEPES, and compound/vehicle (0.2% DMSO). Dye solution (prepared according to the manufacturer) was added to the wells and plates are incubated for 1.5 h light-protected in room temperature. The Tecan Spark plate reader injectors were then primed with capsaicin and Ionomycin for channel activation and data normalization, respectively. Compounds effects were compared to vehicle control, and AMG9810 and NH91 positive controls. To calculate IC50 values, the data was fitted to sigmoidal regression line using Prism GraphPad. The fitting was constrained to a minimum of 0 and a maximal response of about 1 unless a different maximal response could be identified.

### TRPV1 Electrophysiology:

Extracellular (Bath) solution: The solution was composed of (in mM): NaCl, 140; KCl, 4; CaCl₂, 1.8; MgCl₂, 1.2; Glucose, 11; HEPES, 5.5. pH was adjusted to 7.3 with NaOH. Osmolarity was adjusted to 310 mOsm with Sucrose.

Intracellular (Pipette) solution: Pipettes were pulled from borosilicate glass (Warner Instrument Corp, USA) with a resistance of 3-7 MΩ and filled with internal solution composed of (in mM): KCl, 130; MgCl₂, 1; K ATP, 5; EGTA, 5; HEPES, 10. pH was adjusted to 7.3 with KOH. Osmolarity was adjusted to 290 mOsm with Sucrose. A programmable valve-linked pressurized perfusion system was used to locally apply compounds nearby the cell recorded, in a consistent flow rate of 2-3 ml/min. Data were sampled at 5 kHz and low pass filtered at 2.4 kHz using MultiClamp 700B amplifier with pCLAMP11 software (Molecular Devices, USA).

Membrane potential was held in −60 mV. To measure the inhibitory effects of our test compounds on hTRPV1 activity, hTRPV1 gating was achieved by fast short applications of hTRPV1 activator Capsaicin (100 nM, 6 s), with or without compound (>3 minutes) co-application. A 3-minute time interval was set between sequential Capsaicin injections to allow its washout and cell recovery. All compounds are injected in the vicinity of the cells using the pressurized perfusion system.

The drug current response, when capsaicin (co-applied with test compound) is injected following >3 minutes of pre-incubation with the test compound, is divided by the averaged control currents, obtained when capsaicin is injection alone, before compound incubation and following its washout, to evaluate the drug/control response.

For the study of TRPV1 pH-activation, the following pH 5.5 MES based extracellular activation solution is used:

Extracellular (bath) pH 5.5 activation solution: The solution was composed of (in mM): NaCl, 140; KCl, 4; CaCl₂, 1.8; MgCl₂, 1.2; Glucose, 11; MES, 5.5. pH was adjusted to 5.5. Osmolarity was adjusted to 310 mOsm with Sucrose.

### TRPV1 Dual Activity:

### Assessing TRPV1 Dual-Activity Using the FLIPR Potassium Assay Kit:

Cell culture and DNA transfection: CHO/hKv7.2/3 cells were transfected with hTRPV1 DNA construct (CDS of NM_080704.4, inserted into the multiple cloning site of pcDNA3.1(+) between the Hind III and BamH I restriction site, Genscript). For CHO/hKv7.2/3 transfection, 1×10⁶ cells were suspended in a reaction mix (Amaxa™ 4D-Nucleofector™-LONZA) containing 5 μg of DNA for each transfection in a cuvette. The cells were transfected (Amaxa, DT-133 program for CHO transfection with p-3 Kit) and recovered after 10 minutes in room temperature (RT) in the hood and gently transferred with Lonza pipette dropwise into three wells of 6-wells containing prewarm medium (antibiotic-free) to settle down. 24 hours following transfection, the medium was changed to contain the stable cell line selection antibiotics.

According to the optimization process, to achieve a uniform and consistent screening conditions, 24 hours before the assay conductance (48 hours following transfection), the cells were plated in a black, flat 384-well plate (Greiner #781091) at a density of 5000 cells per well and incubated overnight in their normal growth medium.

High content screen (HCS) of hTRPV1 dual-activity using the FLIPR Potassium Assay Kit: The screening of molecules against CHO/hKv7.2/3 cells transiently transfected with hTRPV1 ion channel was performed using the Potassium Assay Kit (R8222 FLIPR Potassium Assay Explorer Kits, Molecular Devices) based of the fact that hTRPV1 is a nonselective cation ion channel and therefore also conducts Tl⁺ upon gating. Thus, the intensity of the Tl+ signal is proportional to the number of hTRPV1 channels in the open state. Therefore, the kit provides a functional indication of the hTRPV1 channel activity.

On the experiment day, the medium was replaced with HBSS, HEPES, and the test compound/vehicle (0.2% DMSO). Dye solution (prepared according to the manufacturer) was added to the plates, which were incubated for 1.5 hours, light-protected at room temperature. Tecan Spark reader plate injectors were primed with Tl⁺+K⁺ (1 mM Tl₂SO₄; 5 mM K₂SO₄) and Capsaicin (5 nM final concentration) for channels activation and data correction of Kv7.2/3 and TRPV1, respectively.

To measure TRPV1 gating in its dual-activity mode of action (MoA), first, the voltage-gated Kv7.2/3 channels were stimulated with a mixture of K⁺ and Tl⁺ to depolarize the cell membrane (1 mM Tl₂SO₄; 5 mM K₂SO₄). After Kv7.2/3 activity was assessed TRPV1 was stimulated through Capsaicin injection (reaching a final concentration of 5 nM). This degree of TRPV1 activity was compared in cells pre-incubated with different concentrations of the testes compounds as well as in the control group, to thereby obtain a drug over control plots of the different compounds and asses their IC50 values.

Assessing hTRPV1 Dual Activity Using Electrophysiology:

Cell culture and DNA transfection: CHO/hKv7.2/3 cells were transfected with hTRPV1 DNA constructs (CDS of NM_080704.4, inserted into the multiple cloning site of pcDNA3.1(+) between the Hind III and BamH I restriction site, Genscript). For CHO transfection, 1×10⁶ cells were suspended in a reaction mix (Amaxa™ 4D-Nucleofector™-LONZA) containing 5 μg of hTRPV1 DNA and 0.5 μg of GFP DNA (for cells selection) in a cuvette.

The cells were transformed (Amaxa, DT-133 program for CHO transfection with p-3 Kit) and recovered for 10 minutes at RT in the hood. Then cells were re-suspended in a 10 mL antibiotic (Ab)-free medium suspension and were plated on Gelatin or PLL-coated cover glass in different cell densities. On the next morning, the medium was changed to full medium containing the CHO/hKv7.2/3 cell line selection antibiotics.

Extracellular (Bath) solution: The solution was composed of (in mM): NaCl, 140; KCl, 4; CaCl₂, 1.8; MgCl₂, 1.2; Glucose, 11; HEPES, 5.5. pH was adjusted to 7.3 with NaOH. Osmolarity was adjusted to 310 mOsm with Sucrose.

Intracellular (Pipette) solution: Pipettes were pulled from borosilicate glass (Warner Instrument Corp, USA) with a resistance of 3-7 MΩ and were filled with internal solution composed of (in mM): KCl, 130; MgCl₂, 1; K ATP, 5; EGTA, 5; HEPES, 10. pH was adjusted to 7.3 with KOH. Osmolarity was adjusted to 290 mOsm with Sucrose.

A programmable valve-linked pressurized perfusion system was used for local application of compounds nearby the cell recorded in a consistent flow rate of 2-3 mL/minute. Series resistance was corrected and data were sampled at 5 kHz and low pass filtered at 2.4 kHz using MultiClamp 700B amplifier with pCLAMP11 software (Molecular Devices, USA).

At least 48 hours following cell transfection and plating, cells were recorded to assess the TRPV1 dual activity. Membrane potential was held at −90 mV. To measure the inhibitory effects of the tested compounds on hTRPV1 dual activity the following protocol was performed:

Cells were perfused with vehicle-only containing bath solution. After 30 seconds, a single −40 mV 1.5 second step was applied for Kv7.2/3 activation and 30 seconds later capsaicin (1 μM) was applied for 6 seconds to activate the TRPV1.

Cells perfusion was changed to a compound-containing bath solution, and after 2 minutes and 30 seconds of compound application, a single −40 mV 1.5 second step was applied for Kv7.2/3 activation and 30 seconds later capsaicin (1 μM) was applied for 6 seconds to activate the TRPV1.

Cells perfusion was thereafter changed back to vehicle only-containing bath solution. After 30 seconds, a single −40 mV 1.5 step was applied for Kv7.2/3 activation and 30 seconds later capsaicin (1 μM) was applied for 6 seconds to activate the TRPV1 followed by a capsaicin wash out.

The drug current response, when capsaicin was injected following 3 minutes of pre-incubation with the test compound, was divided by the averaged control currents, induced without compound, before compound incubation and following its washout, to evaluate the drug/control response.

### Human NPCs:

Cell culture and differentiation: Human PSC-derived neural progenitor cells (Stem Cell Catalog No. 70901 and 70902) were cultured and expanded on Matrigel coated 6-wells plates, using neuronal progenitor medium containing: Neurobasal media supplemented by non-essential amino-acids, 1%; Glutamax, 1%; B27, 2%, FGF2, 20 ng/mL). hNPC were then plated onto PDL and Matrigel coated 12 mm coverglass at a density of about 50,000 cells/well of a 6 well, in DMEM/F12; 10% KSR; 1% P/S and A83-01 (2 μM) from days 0-5. From days 0-9 the medium contained also CHIR99021 (6 μM). From Day 3-9 the medium included RO4929097 (2 μM) and SU5402 (3 μM). From day 9 and onwards, the media change (Neurobasal Media contains: NT-3; BDNF; NGF; GDNF) was performed every other day by replacing only 50% of the media after CO₂ equilibrating. On day 12 cells were incubated with Mitomycin C chemotherapeutic agent (2.5 pg/mL, 2 hours, 37° C.) to avoid glial cells proliferation.

Electrophysiology: To characterize the neuronal properties with electrophysiology recordings, round small neurons (20-40 μm in diameter) were selected from day 31 and onwards. Cells were monitored for their polarized negative membrane potential and for their ability to evoke consistent spikes train responses to repeated positive current injections as a mark of achieving electrical maturation before testing the effect of the tested compounds on neuronal excitability. Membrane excitability was monitored using current clamp. Positive current steps of different amplitude were injected (400 ms) to induce spikes trains before and following compound application.

### Rat DRGs:

rDRGs isolation and primary cell culture: Briefly, rat DRGs from all spinal levels were carefully removed and collected in HBSS on ices, connective tissue of the epineurium surrounding the ganglion was removed and cells were dissociated using Collagenase-II and trypsin dissociation solutions. DRGs were dissociated further, passed through glass Pasteur pipettes to obtain single cells. Then dissociation solution was changed to 5% FBS containing DRG neuronal culture medium and plated onto ECL coated 12 mm cover glass.

Additional rDRGs experiments were performed using Lonza primary neonatal rat (Sprague Dawley neonatal; P2,3) DRG neurons (R-DRG-505, Lonza) plated and cultured according to the manufacturer.

These cryopreserved DRG cells were prepared from freshly isolated and dissociated spinal cord dorsal root ganglia and comprise a normal distribution of neurons and glia (schwann cells).

Electrophysiology: Cells were used for electrophysiological recording at least 48 hours following rDRG isolation or at least 5 days following cryopreserved rDRG thawing. Round small neurons (20-40 μm in diameter) were selected for electrophysiology recordings, to select for small nociceptive neurons that propagate pain sensation in vivo and that are expressing both Kv7.2/3 and TRPV1 targets. Membrane excitability was monitored using current clamp configuration of the whole-cell patch-clamp technique. Positive current steps of different amplitude were injected (400 ms) to induce spikes trains before and following compound application.

To assess the potency of compounds using DRG neurons, the inhibitory potency for CAP-evoked action potential (AP) trains was measured. For this purpose, DRG membrane potential was continuously measured, and the following protocol was performed:

Cells were perfused with vehicle containing bath solution. After 30 seconds, CAP (1 μM) was applied for 6 seconds to activate TRPV1 and stimulate AP trains.

After 30 seconds of perfusion with bath solution containing only vehicle, perfusion was changed to a bath solution containing compound, and after 3 minutes of compound application, CAP (1 μM) was applied again for 6 seconds to activate TRPV1 and stimulate AP trains.

Cells perfusion was changed back to bath solution containing only vehicle and after 3 minutes of compound washout, CAP (1 μM) was applied again for 6 seconds to activate TRPV1 and stimulate AP trains.

To evaluate the drug/control response, the number of AP spikes induced by CAP following incubation with test compound was divided by the average number of control AP spikes induced without compound, before compound incubation, and following its washout.

### Off-Target Assays:

### Kv7.3/5, Kv7.4 Assays:

Cell culture: Chinese hamster ovary (CHO) cell-line cells were cultured in F-12 nutrient mixture (Biological Industries) supplemented with 10% fetal bovine serum with 1% penicillin-streptomycin (Biological Industries) in a humidified 5% CO₂ incubator at 37° C. To express the appropriate gene either Kv7.3/5, Kv7.4 or TRPA1 5 μg plasmid was transfected using Amaxa, DT-133 program, with p-3 Kit.

Kv7.3/5 and 7.4HCS using FLIPR Potassium Assay Kit: Kv7.3/5 and 7.4 HCS using the FLIPR Potassium Assay Kit was performed similarly to the Kv7.2/3 HCS, except that the Tecan Spark plate-reader injectors were primed with Tl+(1 mM Tl₂SO₄) or Tl++K+(1 mM Tl₂SO₄; 5 mM K₂SO₄) for Kv7.3/5, or Tl+(1 mM Tl₂SO₄) or Tl++K+(1 mM Tl₂SO₄; 10 mM K₂SO₄) for Kv7.4 for channel activation and data correction.

FLIPR Potassium Assay Kit: The FLIPR Potassium Assay Kit (R8222 FLIPR Potassium Assay Explorer Kits, Molecular Devices) was used with transfected CHO cells.

According to optimization process, to achieve a uniform and consistent screening condition of molecules, the following protocol was established: 2 days following transfection cells were seeded in 384-well, black-walled, clear-bottomed, at a density of 5,000 cells per well 24 hours before the assay conductance and incubated overnight in their normal growth medium. On the experiment day the medium was replaced with HBSS, HEPES, and the tested compound/vehicle (0.2% DMSO). Dye solution (prepared according to the manufacturer) was added to the wells and plates were incubated for 1.5 hour light-protected in room temperature. The Tecan Spark plate-reader injectors were primed with Tl+(1 mM Tl₂SO₄) or Tl++K+(1 mM Tl₂SO₄; 5 mM K₂SO₄) for Kv7.3/5 channel activation and data correction or Tl+(1 mM Tl₂SO₄) or Tl++K+(1 mM Tl₂SO₄; 10 mM K₂SO₄) for Kv7.4 for channel activation and data correction. Compounds effects were compared to vehicle control and Retigabine positive control (Known IC50=1.5-5 μM). To calculate IC50 values, the data was fitted to sigmoidal regression using Prism GraphPad.

### TRPA1 Assay:

Cell culture: Chinese hamster ovary (CHO) cell-line cells were cultured in F-12 nutrient mixture (Biological Industries) supplemented with 10% fetal bovine serum with 1% penicillin-streptomycin (Biological Industries) in a humidified 5% CO₂ incubator at 37° C. To express the appropriate TRPA1 5 μg plasmid was transfected using Amaxa, DT-133 program, with p-3 Kit.

High content screen (HCS) using Fluo-8 No Wash Calcium flux Assay Kit: Fluo-8 No Wash Calcium Assay Kit (Abcam, ab112129) was used with CHO transiently transfected TRPA1 to screen compounds against the hTRPA1 ion channel target. The cells are pre-loaded with Fluo-8AM which is membrane permeable. The AM groups of the Fluo-8AM are then being cleaved by intracellular esterase, trapping the Fluo8 in the cell. Calcium influx through activated TRPA1 channels significantly increases the fluorescence of Fluo-8. The relative fluorescence signal is calculated following background subtraction, comparing the fluorescence at each time point to its maximal level measured following Ionomycin application in the end.

According to optimization process to achieve uniform and consistent screening condition of molecules the following protocol was established: Cells were seeded 2 days following transfection in 384-well, black-walled, clear-bottomed, at a density of 5,000 cells per well 24 h before the assay conductance and incubated overnight in their normal growth medium. On the experiment day the medium replaced in each well with HBSS, HEPES, and compound/vehicle (0.2% DMSO). Dye solution (prepared according to the manufacturer) is added to the wells and plates are incubated for 1.5 h light-protected in room temperature. The Tecan Spark plate reader injectors are then primed with AITC and Ionomycin for channel activation and data normalization, respectively. Compounds effects are compared to vehicle control, and known inhibitor of TRPA1-A967079. To calculate IC50 values, the data was fitted to sigmoidal regression line using Prism GraphPad. The fitting was constrained to a minimum of 0 and a maximal response of about 1 unless a different maximal response could be identified.

hERG Assay:

Cell culture: Chinese hamster ovary (CHO) cell-line cells with a constitutive expression of human hERG channels (B'SYS GmbH, Switzerland) were cultured in F-12 nutrient mixture (Biological Industries) supplemented with 10% fetal bovine serum (Biological Industries) and 1% penicillin-streptomycin (Biological Industries) in a humidified 5% CO₂ incubator at 37° C. To maintain the hERG expression stability of the CHO cell line, antibiotics selection (G418 200 g/mL and Hygromycin 500) was applied.

FLIPR Potassium Assay Kit: The FLIPR Potassium Assay Kit (R8222 FLIPR Potassium Assay Explorer Kits, Molecular Devices) was used with CHO/hERG cell line, for screening compounds against the hERG ion-channel off-target, as detailed hereinabove.

According to optimization process, to achieve a uniform and consistent screening condition of molecules, the following protocol was established: Cells were seeded in 384-well, black-walled, clear-bottomed, at a density of 7,500 cells per well 24 hours before the assay conductance and incubated overnight in their normal growth medium. On the experiment day the medium was replaced with HBSS, HEPES, and the tested compound/vehicle (0.2% DMSO). Dye solution (prepared according to the manufacturer) was added to the wells and plates were incubated for 1.5 hour light-protected in room temperature. The Tecan Spark plate-reader injectors were primed with Tl+(1 mM Tl₂SO₄) or Tl++K+(1 mM Tl₂SO₄; 10 mM K₂SO₄) for channel activation and data correction. Compounds effects were compared to vehicle control and Terfenadine positive control (Known IC₅₀=200 nM). To calculate IC₅₀ values, the data was fitted to sigmoidal regression using Prism GraphPad.

### HepG2 Assay:

Cell culture: HepG2 cell-line cells were cultured in EMEM (ATCC 30-2033) medium supplemented with 10% fetal bovine serum (Biological Industries) in a humidified 5% CO₂ incubator at 37° C.

ATPlite™ 1step Kit: ATPlite™ 1step kit (PerkinElmer) was used in a 384-wells format to assess potential liver toxicity using HepG2 cells. This is an ATP detection system based on firefly luciferase luminescence for the quantitative evaluation of proliferation and cytotoxicity of cells. ATP is a marker for cell viability because it is present in all metabolically active cells and its concentration declines very rapidly when the cells undergo necrosis or apoptosis.

According to an optimization process done to achieve uniform and consistent screening condition of molecules, the following protocol was established: Cells were seeded in 384-well, black-walled, clear-bottomed, at a density of 4000 cells per well 48 hours before the assay and incubated overnight in their normal growth medium. On day two (48 hours before viability measurement), the tested compounds and the positive control were applied to the wells according to the plate design. Compounds were dissolved in phenol-free growth medium at 0.1% DMSO concentration and their concentrations were identified using a solubility assay as described herein. 48 hours later, reconstituted lyophilized substrate solution was added to each well and the sealed 384-well microplate was mixed for 2 minutes at 1100 rpm in an orbital microplate and plates were incubated for 10 minutes in the dark at room temperature. Right after, luminescence was measured in the Tecan plate reader. To estimate the LD50 and LD25 values the data was fitted to sigmoidal regression using Prism-GraphPad. Takeda's TAK243, a toxic small-molecule inhibitor of the ubiquitin-activating enzyme with toxicity IC50 of about 200 nM, was used as the positive control.

### Druggability:

### In-Vitro Selectivity Assay:

A broad panel of 78 recombinant targets was tested for pharmacologic activity in vitro through a variety of functional assays (Eurofins DiscoverX, USA), as part of the National Institute of Neurological Disorders and Stroke (NINDS) Preclinical Screening Platform for Pain (PSPP) program. The biological targets for the in vitro profiling have been selected based on avoiding opioid receptor stimulation and targets known to be associated with adverse events or targeted by drugs of abuse. Briefly, compounds were tested at concentrations up to 10 μM, or to their solubility limit, if less. For each test, a reference compound was evaluated at the target of interest as a positive control.

### Metabolic Stability and SAR Driven Optimization of Human Liver Microsome (HLM) Values:

Metabolic stability refers to a compound susceptibility to biotransformation. Both in vitro half-life (t_{1/2}) and intrinsic clearance (CLint) are typically utilized to express metabolic stability. CLint represents the maximum activity of liver microsomal proteins/hepatocytes towards a compound, without involving other physiological parameters such as hepatic blood flow and drug binding within the blood matrix. Since oral drugs first pass metabolism is via the liver the CLint parameter is an important factor.

Liver microsomes which are subcellular particles derived from endoplasmic reticulum of hepatic cells were used for evaluating the tested compounds. These particles are rich in drug metabolizing enzymes, including the cytochrome p-450 family. Liver microsomes are a recommended test system for various in vitro drug metabolism and pharmacokinetics (DMPK) studies. CLint values lower than 100 l/min/mg are considered suitable for drug development.

Liver Microsome Metabolic Stability Assay (NADPH): The liver microsome stability assay was evaluated through pre-incubation with HLMs. A liver microsome mixture (pooled from multiple donors) at a concentration of 0.5 mg/mL, and the test compound at a concentration of 1 μM, were warmed in the presence of a NADPH regenerating system (about 1.0 mM) at 37° C. Positive controls, including testosterone (a CYP3A4 substrate), propafenone (a CYP2D6 substrate), and diclofenac (a CYP2C9) substrate, were concurrently incubated with the HLMs, in the presence of the NADPH regenerating system (1.0 mM). Samples were extracted during incubation at multiple intervals: 0, 5, 15, 30, 45 and 60 minutes. To terminate the reactions, the samples were promptly mixed with cooled acetonitrile that included an internal standard (IS). An additional compound was incubated with the HLMs in the absence of the NADPH regeneration system, from which a sample was taken at the 60-minute interval.

The clearance of the test compound in the samples was subsequently analyzed by LC-MS/MS based on the peak area ratios of the analyte to the IS.

The following equation was used to calculate the microsome clearance:

$$C \text{?} = {C}_{0} \cdot e \text{?}$$

$${T}_{1 / 2} = \frac{Ln 2}{- k \text{?}} = \frac{0.693}{- k \text{?}}$$

$${CL}_{int ( mic )} = \frac{0.693}{In vitro {T}_{1 / 2}} \cdot \frac{1}{mg / mL microsomal protein in reaction system}$$

$$\text{?} \text{indicates text missing or illegible when filed}$$

Liver weight: 40 grams/kg, 30 grams/kg, 32 grams/kg, 20 grams/kg and 88 grams/kg, for rat, monkey, dog, human and mouse, respectively.

CLint(mic) was used to calculate the liver clearance:
mg microsomal protein/gram liver weight: 45 mg/gram for the species

$${CL}_{int ( liver )} = {CL}_{int ( mic )} \cdot \frac{mg microsomes}{g liver} \cdot \frac{g liver}{kg body weight}$$

### Log D Measurements:

Log D, a Log of partition of a chemical compound between a lipid phase and an aqueous phase, was measured at pH 7.4 (which equals Log P), in order to calculate accurately the LLE and to uncover compounds that exhibit improved physiochemical properties.

The Log D assay was a miniaturized 1-octanol/buffer shake flask method followed by LC/MS/MS analysis, as follows.

The test compounds or control compounds Nadolol, Chlorpromazine Hydrochloride and Propranolol Hydrochloride were dissolved in 100% DMSO to obtain 10 mM solutions, which were transferred to 96-well polypropylene cluster tubes; 2 μL/well. 1-octanol saturated with 0.1 M phosphate buffer (149 μL/well) and 0.1 M phosphate buffer saturated with 1-octanol (149 μL/well) were added to the tubes, respectively, and each tube was vigorously mixed for 2 minutes and then shaked at a speed of 800 rpm at room temperature for 1 hour, followed by centrifugation at 4000 rpm for 5 minutes at room temperature. Thereafter, for the test compounds and the Chlorpromazine Hydrochloride and Propranolol Hydrochloride, the sample of the buffer layer was diluted by a factor of 50-fold and the sample of 1-octanol layer was diluted by a factor of 2000-fold with internal standard (IS) solution. For Nadolol, the sample of the buffer layer was diluted by a factor of 1000-fold and the sample of 1-octanol layer was diluted by a factor of 400-fold with internal standard (IS) solution. The samples were analyzed using a triple quadrupole mass spectrometer. Peak areas were corrected by dilution factors and embedded internal standard, and the ratio of the corrected peak areas was used to calculate the Log D value. The Log D value for each compound will be calculated by the following equation:

$$Log {D}_{octanol / buffer} = log 10 ( \frac{\begin{matrix}Mean Octanol Layer Peak Area Ratio * \\ Octanol Layer Dilution Factor\end{matrix}}{\begin{matrix}Mean Buffer Layer Peak Area Ratio * \\ Buffer Layer Dilution Factor\end{matrix}} )$$

### LLE Measurements:

Ligand-lipophilicity efficiency (LLE) is a parameter used in drug design and drug discovery to evaluate the quality of research compounds, linking potency and lipophilicity.

To follow LLE values, the following equation was used:

LLE=−Log (IC50)−c Log P

Suitable LLE value are typically higher than 3 or higher than 5.

### CYP (Cytochrome P450) TDI (Time-Dependent Inhibition) Assay:

The time-dependent inactivation potency of the test compound to CYP enzymatic activities was assessed by the pre-incubation of the human liver microsomes with the test compound in the presence and absence of NADPH, followed by the incubation with the discrete marker substrates, as follows.

Pre-incubation with human liver microsomes (HLMs): The TDI inactivation potency on CYP enzymatic activities was evaluated through pre-incubation with HLMs (Corning). A liver microsome mixture at a concentration of 0.1 mg/mL, and the test compound at 8 different concentrations (0, 0.05, 0.165, 0.5, 1.65, 5.0, 16.5, or 50.0 μM) was warmed at 37° C. for 10 minutes. Concurrently, a set of known time-dependent inactivators was also prepared in duplicate for comparison. Following this, NADPH (1.0 mM) was added to one group (Group 1), and an equal volume of potassium phosphate buffer to the second group (Group 2), marking the beginning of a 30-minute pre-incubation at 37° C.

Incubation: After pre-incubation, the marker substrate, or marker substrate plus NADPH was added to the mixture, thereby initiating the incubation. The duration of the incubation was dependent on the type of CYP isoform being evaluated. For example, with the CYP3A isoform, the mixture was incubated over 3 minutes utilizing midazolam as the marker substrate (2.0 μM), verapamil as the standard inactivator (3.5 μM).

Termination of Reaction: Following the incubation, ice-cooled acetonitrile containing the internal standard (IS) was added to halt the reactions. Metabolites generated from the marker substrates were then measured via LC-MS/MS, assessed based on peak area ratios of the analyte to IS.

### Met-ID (Metabolite Identification) Assay:

Cell culture and incubation: Cryopreserved hepatocyte cells, from human and rat sources, were purchased from commercial sources or in-house production, and were cultured in Williams' Medium E supplemented with glutamine and HEPES, in a humidified 5% CO₂ incubator at 37° C., to a cell density of 0.0×10⁶ cells/mL. Then, solutions of test compounds at a concentration of 10 μM, or 7-Ethoxycoumarin, as a positive control, at a concentration of 30 μM, in Eagle's Minimum Essential Medium, Catalog No. 30-2003 (ATCC) supplemented with 20% fetal bovine serum, were introduced and the mixture was incubated for 120 minutes, or other specified time points.

The samples were analyzed for metabolites using an LC-MS method developed on FIRMS and PDA systems. Comparative analyses of mass spectra and ion fragmentation between the parent compound and its metabolites were carried out, and metabolic profiling was determined using the mass spectrometric and/or PDA (UV) response. Hypothetical structures of metabolites and their metabolic pathways were proposed.

### Bidirectional Permeability in Caco-2 Cells:

Cell culture: Caco-2 cells (ATCC) were seeded onto polycarbonate (PC) membranes in 96-well insert plates. The cells were cultured in media for 21-28 days prior to performing the transport assays.

Validation of monolayer integrity: Once the Caco-2 cells formed a confluent monolayer, their integrity was evaluated using a Lucifer yellow rejection assay. The monolayer integrity was further evaluated by measuring, in duplicate wells, the unidirectional (A→B) permeability of nadolol (a low permeability marker) and metoprolol (a high permeability marker), as well as bidirectional permeability of digoxin (a P-glycoprotein substrate marker).

Assay: The compounds were assayed under bidirectional transport conditions (A→B and B→A) at a concentration of 2.00 μM (with DMSO≤1.0%), using a transport buffer composed of HBSS containing 10.0 mM HEPES, pH 7.40±0.05. The incubation was carried out at 37° C., with 5.0% CO₂ and relatively saturated humidity for 2 hours.

The dosing solution was spiked and mixed with the transport buffer and a Stop Solution (containing an internal standard (IS)) to create a T₀ sample.

Sample analysis: Following the incubation, all of the samples were analyzed using LC-MS/MS. The concentrations of the test compounds were expressed as peak area ratios of the analytes versus the IS.

### Plasma-Protein Binding Assay (HTD Method):

Incubation: Test compounds or the positive control warfarin were introduced into 10% plasma samples (Sourced from WuXi DMPK labs or from commercial vendors) to a concentration of 2 μM. A portion of 150 μL of the compound-containing plasma sample was added to one chamber of a 96-well equilibrium HTD dialysis plate. An equal volume of dialysis buffer was added to the corresponding chamber. A plasma sample was also taken before the incubation and designated as the T₀ sample, serving for recovery calculation. The plate was incubated in a humidified 5% CO₂ incubator at 37° C. for 4 hours. Each incubation was set up in triplicate.

Sample extraction and preparation: Following the incubation period, samples of 50 μL were drawn from both the plasma side and the buffer side and each was mixed with an equal volume of blank plasma. All matrix-matched samples were quenched using a stop solution containing an internal standard (IS).

Sample Analysis: The samples were subsequently analyzed by LC-MS/MS. The concentrations of the test compound in plasma and buffer samples were quantified based on the peak area ratio of analyte to the internal standard, with no requirement for a standard curve.

### Rat Pharmacokinetics Assay:

The pharmacokinetic (PK) properties of test compound following single intravenous (IV) bolus and oral gavage (PO) administration in male Sprague-Dawley (SD) rats were determined in this study.

Test compound administration: Six male Sprague Dawley (SD) rats were divided into two designated groups, Group 1 and Group 2, each composed of three rat subjects. Group 1 animals were subjected to a single intravenous bolus administration of the test compound at a dosage of 1 mg/kg. Group 2 animals were administered with the test compound orally in a single dose, at a concentration of 10 mg/kg. Throughout the study, the animals had unhindered access to food. The test compound was administered at a concentration of 0.5 mg/mL and dose volume of 2 mL/kg for Group 1, and at a concentration of 1 mg/mL and dose volume of 10 mL/kg, for Group 2.

Sample extraction and analysis: Plasma samples were systematically collected at varying time points of 0.083 (only for Group 1), 0.25, 0.5, 1, 2, 4-, 6-, 8-, and 24-hours post-dose. LC-MS/MS was used to determine the test compound's concentration within the collected plasma samples. Data reflecting plasma concentration in correlation to time were graphically represented and subjected to a non-compartmental analysis using the Phoenix WinNonlin 6.3 software program. The resulting pharmacokinetic parameters, including Clearance (Cl), Volume of distribution at steady state (Vdₛₛ) and initial concentration (C₀) for intravenous administration; peak concentration (Cₘₐₓ), time of peak concentration (Tₘₐₓ), and bioavailability (% F) for extravascular administration; and half-life (T_{1/2}), Area Under Curve from time zero to time t (AUC₍₀₋ₜ₎), Area Under Curve from time zero to infinity (AUC_{(0-inf)}), Mean Residence Time from time zero to time t (MRT₍₀₋ₜ₎), Mean Residence Time from time zero to infinity (MRT_{(0-inf)}) for all routes, were systematically calculated.

The experimental design is summarized in the following table:

| Group | No. of | | Dose | Dose | Conc | Dose Volume | |
|---|---|---|---|---|---|---|---|
| No. | animals | Sex | Route | (mg/kg) | (mg/mL) | (mL/kg) | Vehicle |
| 1 | 3 | Male | IV bolus | 1 | 0.5 | 2 | 5% DMSO/95%(20% HP- |
| | | | | | | | β-CD in water ) |
| 2 | 3 | Male | PO | 10 | 1 | 10 | 5% DMSO/95%(20% HP- |
| | | | | | | | β-CD in water ) |

### In-Vivo Efficacy and Safety:

All animal experiments were carried out according to the Guide for the Care and Use of Laboratory Animals (2011 edition) and approved by the Israel National Institutional Animal Care and Use Committee (IACUC). Young adult male SD rats (Envigo RMS, Israel), were used for in vivo efficacy: Spared Nerve Injury (SNI), Distal Tibial Injury (DTI) and Post-operative Pain (POP), 200-220 grams, Chronic Post-Ischemia Pain (CPIP) 275-325 grams, Monosodium iodoacetate-induced osteoarthritis (MIA-OA) 180-220 grams; for in vivo safety: 200-250 grams at study initiation. The weight variation of animals at the time of treatment initiation did not exceed ±20% of the mean weight. The health status of the animals used in this study was examined upon their arrival. Only animals in good health were acclimatized to laboratory conditions and were used in the studies. All studies were performed following at least five days of animal acclimation. During acclimation and throughout the entire study duration, animals were housed within a limited-access rodent facility and kept in groups with a maximum of 2-3 rats per polypropylene cage. The cages were fitted with solid bottoms and filled with sterile wood shavings as bedding material. Animals were provided ad libitum with a commercial, sterile rodent diet and had free access to drinking water that was supplied to each cage via polyethylene bottles with stainless steel sipper tubes. A feed lot analysis of the diet batch used in the study was included in the archives with the study data. Water was monitored periodically. Plastic cylinders for playing and hiding and wooden balls or sticks were added to the cages for environmental enrichment.

Automatically controlled environmental conditions were set to maintain the temperature at 17-25° C. with relative humidity (RH) of 30-70%, a 12:12 hour light: dark cycle and 10-15 air changes/h in the study room. Temperature and RH were monitored daily. The light cycle was monitored by the control clock. Each dosing group was kept in separate cages to avoid cross-contamination, which could occur through the consumption of fecal matter. At the end of the study the remaining animals were euthanized with pentobarbital sodium solution IP.

### Spared Nerve Injury (SNI) and Distal Tibial Injury (DTI):

SNI: The SNI procedure consisted of ligating and dissecting the tibial and common peroneal nerves, thus sparing the sural nerve. Prior to surgery, a dose of 0.03 mg/kg buprenorphine (Temgesic®) was administered to animals as a prophylaxis to alleviate the operative and postoperative pain unrelated to neuropathy. Next, animals were anesthetized with a combination of medetomidine/ketamine sodium IP. While under anesthesia a medio caudal incision of 4-5 mm parallel to the left femur was cut on shaved and disinfected skin. The sciatic nerve was exposed from underneath the muscle layer and isolated gently from the surrounding connective tissue, using curved and blunt forceps. The tibial and common peroneal nerves were first tightly ligated with double knot of 6-0 silk suture, proximally and immediately distal to the sciatic bifurcation. Thereafter, the nerves which run as a pair as physically attached to each other were cut at sites close to the ligatures, thereby removing a length of approximately 0.5 mm of the nerves, necessary to avoid the formation of any re-connection to the nerves. This procedure led to complete denervation of areas of tibial and peroneal innervation in the plantar paw, while neuropathy was developed upon the sural territories. Special care was taken not to manipulate the sural nerve, which thus was left intact. The muscle layers and skin were sutured, and animals were allowed to recover. 0.03 mg/kg buprenorphine SC was administered on postoperative days 1-2, every 12 hours, starting from the evening of Day 0. Rehydration with 4 ml of sterile saline IP was provided directly after the surgery, continuing twice a day for 7 days. The sural nerve innervates the lateral longitudinal third of the plantar paw and therefore, mechanical sensitivity was assessed in this area.

DTI: The DTI procedure was conducted similarly to the SNI model except for two modifications: First, in DTI, only the tibial nerve was cut, leaving the sural and the peroneal nerves intact. Second, the DTI model was a distal, not proximal, injury. The tibial nerve was first exposed proximally to the ankle. Thereafter, the nerve was cut, removing a length of approximately 2 mm of the nerve to avoid the formation of any re-connection to the nerves.

The selection of animals was performed on day 13 post-operation before animals were placed in their experimental groups. Animals with a pain threshold of <4 grams for an operated leg following Von Frey testing were included in the study. To form homogenous treatment groups, all the selected animals were grouped using a random sampling method on study day 13, to form groups with similar pain threshold mean. For blinding purposes, the researcher who dosed the animals with test items was different than the researcher who conducted the VF tests.

Post-operative Pain (POP): For this model, animals were anesthetized by medetomidine/ketamine sodium IP. Under anesthesia, a 1 cm longitudinal incision over the plantar surface of the left hind paw was performed, and the plantaris muscle was incised longitudinally. Following the post-operative pain (POP) surgery, the incision was closed with two stitches, and the rats were then allowed to recover from general anesthesia for about 1 hour. On the following day, animal responses to von Frey (VF) filaments and hot-plate testing were recorded.

Chronic Post-Ischemia Pain (CPIP): The CPIP model was generated following exposure to prolonged hind paw ischemia and reperfusion. On study day 0, animals were anesthetized over 3 hours with a bolus of pentobarbital sodium (50 mg/kg; IP) and chronic IP infusion of pentobarbital sodium for 2 hours (20 mg/kg/hour). After induction of anesthesia, a Nitrile 70 Durometer O-ring with 5.5 mm internal diameter was placed around the rat's left hind limb just proximal to the ankle joint. The O-rings were selected to produce a tight-fit that induces ischemia similar to that which occurs by inflating to 350 mmHg blood pressure cuff and left on the limb for 3 hours. The position of the O-ring was on the limb just proximal to the medial malleolus, by sliding the O-ring off the outside of a 3 cm syringe (cut in half) after the hind paw was inserted into the syringe barrel as far as possible.

On study day 13 following the CPIP operation, the animals' response to von Frey was measured, and animals that exhibited a withdrawal threshold of less than 6 grams were included in this study. On study day 14, the animal's response to von Frey filaments was measured.

Monosodium iodoacetate-induced osteoarthritis (MIA-OA): The animals were anaesthetized using an Isoflurane+O₂ mixture. Then 50 μl of MIA at a concentration of 60 mg/mL was injected into the right knee joint using a 30 G needle. On study days 13 and 14 following MIA injection, the animals' dynamic weight bearing and response to von Frey filaments were measured.

Mechanical Allodynia (Von Frey): The rats were placed in an enclosure and positioned on a metal mesh surface but allowed to move freely. The rats' cabins were covered with red cellophane to diminish environmental disturbances. The test begun after the cessation of exploratory behavior. Von Frey TouchTest sensory evaluator (North Coast Medical, CA, USA) was used with the following monofilaments (force in g): 4.31 (2 grams); 4.56 (4 grams); 4.74 (6 grams); 4.93 (8 grams); 5.07 (10 grams); 5.18 (15 grams); 5.46 (26 grams). The testing started with the smallest filament of 4.31 (2 grams) force.

Four stimulations were applied in different paw locations. If the rat lifted its leg as a result of any of these stimulations, a withdrawal response was considered as the value of reference. If there was no clear response to a filament after four tries, the next higher filament was used.

Hot Plate: Animals were placed on hot plate analgesia meter (IITC Life Science Inc., CA, USA) set to 53±1° C., and the time until the first escape response was observed was recorded. A response was considered when the animals lifted or licked their leg or performed kicking behavior.

Dynamic Weight Bearing (DWB): DWB study was performed using the DWB2 apparatus (Bioseb, Paris, France). The rats were placed in a transparent cage containing a matrix comprising around 2000 high-precision force sensors embedded in its floor where they were allowed to move freely. The force sensors measured the weight distribution on each of the four paws of the animal in grams. The animal was filmed from above using a high-definition camera, and the video feed was analyzed in real-time during the test using tracking software, allowing a precise analysis of the animal's posture and hind paws identification. Out of 5 minutes of animal behavior recording, a segment of 2 minutes was analyzed. Once the analysis was completed, the software provided the weight distribution of each hind paw as the main readout and the ratio between the injured and intact hind paws was then calculated.

Core Body Temperature: Following two days of habituation to the measurement method, the body temperature was measured on study day for baseline values and then again post-dosing in the time points indicated in the figure. Body temperature was measured using a probe gently inserted into the animal anus using a corn oil lubricant (C8267, Sigma-Aldrich, IL).

Rotarod: There were two days of training in which rats were trained at a constant speed of 5 RPM for 180 seconds. After each fall the rats were placed back on the rotating rod until 180 seconds were reached. Then, the actual test started on study day 0 using the accelerating rotarod methodology. Animals were placed on the rod, which was accelerated 5-40 RPM over 180 seconds. Acceleration rate (0.19 RPM/second) was calculated from delta velocity (v2−v1) divided by delta time (t2−t1). The time to the first fall of each animal was documented, in addition to the distance and speed. The rotarod measurements were day 0 at pre-dosing for baseline and then at the time points indicated in the figure.

### In-Vivo Safety Studies Performed by the PSPP:

### Rotarod Assay:

Animals: 50 male and 50 female Sprague Dawley rats from Envigo (Frederick, MD) were used in the study. Male animals were received at body weights between approximately 200-215 grams, and female animals between 175-205 grams. All animals were housed 3 per cage (15.55×13.63×8.39 inches) and acclimated to the vivarium for 6-9 days prior to testing. All cages contained BedO'Cob® bedding with Nylabones® for enrichment. All rats were examined, handled, and weighed prior to initiation of the study to ensure adequate health and suitability. During the study, 12/12 light/dark cycles were maintained. The room temperature was maintained between 2° and 23° C. with relative humidity maintained around 50%. Chow (LabDiet 5001, LabDiet, St. Louis, MO, USA) and water were provided ad libitum except where specified below. Testing was performed during the animal's light cycle phase (between 6 am and 6 μm). Body weights were recorded prior to baseline assessment. Latency to fall off the rotating rod was the primary measure of performance.

Training: Each trial on the rotarod was identical, a 5 second ramp from 0-17 RPM followed by a period of constant velocity at 17 RPM for up to 40 seconds, for a total trial time of 40 seconds.

Animals ran three training trials on Day 1 with a 30 minutes interval between training trials. During Day 1 training trials, animals received extra guidance to stay on the rod and avoid falls due to turning around or other exploration. Following Day 1 training trials, food was removed from the home cages and all animals were fasted overnight. On Day 2, animals ran a baseline trial prior to dosing and the testing time points. The outcome measured was latency to fall off the rotating rod. Animals that maintained posture and stayed on the rotarod throughout the duration of the trial were given a score of 40 seconds (the maximum possible score). To proceed to the testing phase animals must have achieved a score of 40 seconds during the Day 2 baseline trial. Animals that failed to reach a 40 seconds baseline score on Day 2 were excluded.

Testing: Animals that met inclusion criteria from the Day 2 baseline trial were randomized across treatment groups based on body weight. Animals were then dosed accordingly and run through test trials at predetermined intervals as specified in the test orders. For this experiment those test trials were run 1, 4, and 6 hours following compound dosing.

In this study, 60 males were trained, 55 met inclusion criteria, and 50 were assigned to treatment groups. 60 females were trained, 57 met inclusion criteria, and 50 were assigned to treatment groups.

### Modified Irwin Test:

Twenty male and twenty female Sprague Dawley rats from Envigo were used in the study. Animals were received at approximately 200-250 grams, housed 3 per cage (15.55×13.63×8.39 inches), and acclimated to the vivarium for 6-9 days prior to testing. Rats were housed in cages containing BedO'Cob® bedding with Nylabones® for enrichment. All rats were examined, handled, and weighed prior to initiation of the study to ensure adequate health and suitability. During the study, 12/12 light/dark cycles were maintained. The room temperature was maintained between 2° and 23° C. with a relative humidity maintained around 50%. Chow (LabDiet 5001, LabDiet, St. Louis, MO, USA) and water were provided ad libitum except where specified below. The test was performed during the animal's light cycle phase (6 am to 6 μm), with observations targeted to be carried out between 8 am and 2 μm. Each vehicle or dose group contained 8 rats, 4 males and 4 females.

The day prior to assessment, rats were single housed in clean rat polycarbonate cages and food was removed. Animals were allocated to different groups based on body weight, under the concurrent restriction that cage mates could not be in the same treatment groups. A staff member not involved in behavioral observations allocated animals to experimental groups and assigned each animal a letter code.

For the study, one naïve untreated male and female rat were used as reference to aid in scoring. Prior to the assessment, all rats received two sessions of handling to help in habituation.

On the day of assessment, rats were scored on 39 behaviors (not shown). 1 hour prior to dosing, water was removed from the home cage baseline (BL) and behavioral observations were recorded. Two observers (referred to as Reviewer No. 1 and Reviewer No. 2) with high inter-rater reliability (difference of 2.11%) simultaneously recorded observations at all time points.

Following baseline observations, an independent experimentalist dosed the animals with compound or vehicle, and observations were made at 1, 2, 4, 6, and 24 hours post dose. Ten animals were assessed per day over 4 days. One male and one female subject from each treatment group was assessed by the observers in a pre-determined order that differed each day. All dosing and observations were performed in a dedicated observation room (distinct from the colony).

### Assessment of Individual Behaviors and Number of Observations:

In order to summarize the specific behaviors and the degree of change from normal, a Severity Score was generated using the formula: Severity Score=(Summed Score/Maximum Score)×100. The summed score was the absolute value for each behavior summed across all animals (male and female) at each timepoint. The Maximum Score referred to the score received if all 8 animals at a given dose exhibited a particular behavior at its most extreme. Based on this formula, a score of 100 is the highest severity score that can be derived for each behavior.

While the Severity Score gives an overview of which behaviors were affected, the direction of the behavior is not indicated (e.g., increased, or decreased locomotor activity). To identify which behaviors were observed and the direction of the change, the number of observations (possible total of 16 behaviors) for each behavior and the direction of the change were summarized.

Herein throughout, Compound 627 is also referred to interchangeably as Compound BS627 or Compound BSEN627.

Herein throughout, Compound 661 is also referred to interchangeably as Compound BS661 or Compound BSEN661.

Herein throughout, Compound 760 is also referred to interchangeably as Compound BS760 or Compound BSEN760.

Additional experimental methods and protocols are described hereinunder.

### Example 1

### Structure Activity Relationship (SAR) Studies

As discussed hereinabove, one of the most potent compounds disclosed in WO 2019/073471 is referred to therein as NH91.

NH91 features the following biological and physicochemical parameters:
TRPV1 IC50: 8 μM
Kv 7.2/7.3 EC50: 0.7 μM
C Log P: 5.3
LLE TRPV1: −0.35
LLE Kv 7.2/7.3: 0.8
Solubility: 20 μM With the aim at improving the pharmaceutical performance of NH91, four pharmacophoric sites were marked for possible modifications: Ring A, the bridging amine, Ring B and the side chain (meta to the amine bridge), as shown in Scheme 1 below.

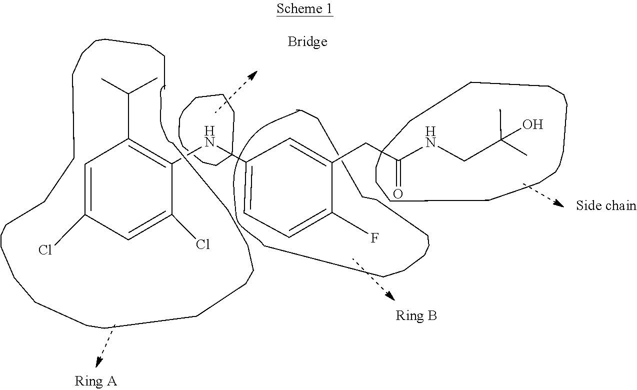

A preliminary library of several dozens of compounds was synthesized and biological measurements and calculations (TRPV1 IC50, Kv7.2/7.3 EC50, LLE, Kinetic solubility, etc.) were performed as described herein.

The preliminary library included the following modifications:

Ring A modifications: replacing the isopropyl group by other groups, such as tert-butyl, cyclopentane, pyridine, etc.; or replacing one of the chloro substituents by e.g., other halo substituents, electron-withdrawing substituents, etc.

Bridge modifications: replacing the amine bridge by sulfonamide or an ether moiety (—O—).

Ring B modifications: replacing the phenyl by pyridine.

Side chain modifications: replacing the linear hydroxyalkyl by a hydroxy-substituted cycloalkyl or heteroalicyclic, or by a branched hydroxyalkyl.

The data obtained for the compounds of this preliminary library (not shown) indicated that:
(i) Introducing bulky hydrophobic substituents on Ring A, such as tert-butyl and cycloalkyls, improved activity on both channels but no improvement was observed in LLE
(ii) Introducing bulky hydrophilic substituents on Ring A, improved LLE but resulted in reduced activity
(iii) Replacing the amine bridge by sulfonamide resulted in substantial drop in the potassium channel opening
(iv) Replacing the amine bridge by an ether moiety resulted in improved parameters but reduced solubility
(v) Replacing Ring B by pyridine resulted in reasonable parameters
(vi) Out of the tested structures, 35 were side chain modifications, since previous studies have shown that both TRPV1 and Kv7.2/7.3 are sensitive to synthetic modifications at this portion of the molecule. The obtained data supported a desired effect of these modifications on the LLE values, along with retained activities and lower c Log P values.

Based on the data obtained for the preliminary library of compounds, additional libraries, including additional modifications in one or two of the pharmacophoric sites shown in Scheme 1, were designed, synthesized and tested. Additional designs mainly included replacing the phenyl in Ring A and/or Ring B by a heteroaryl such as pyridine, imidazole or a heteroalicyclic; replacing the amide bridge by —S—, —S(═O)—, —P(OH)—, —CH(OH)—, or —C(═O)—; replacing the Ring A-NH-aniline moiety by a rigid heteroaromatic moiety such as indole; and further modifications of the side chain site.

Based on the data obtained for the additional libraries (not shown), three main chemotypes were shown to exhibit desired properties in terms of dual activity on TRPV1 and the potassium channel, solubility, LLE and stability.

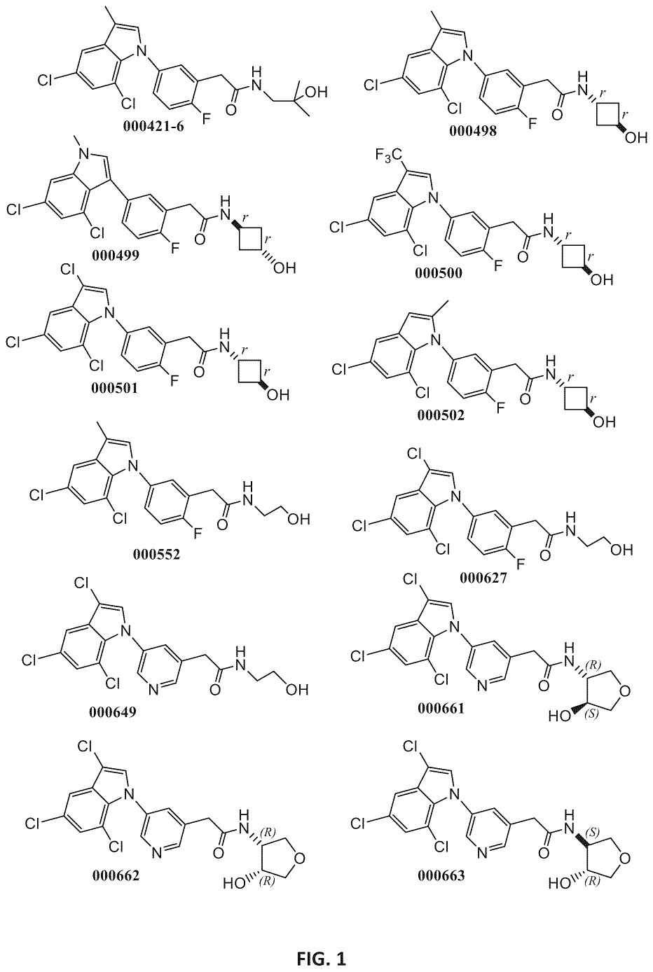

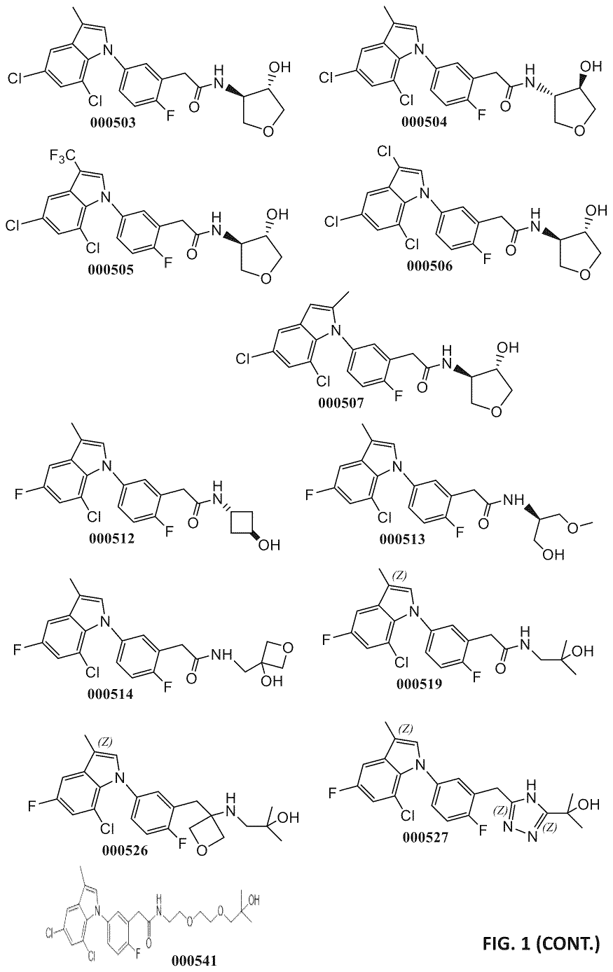

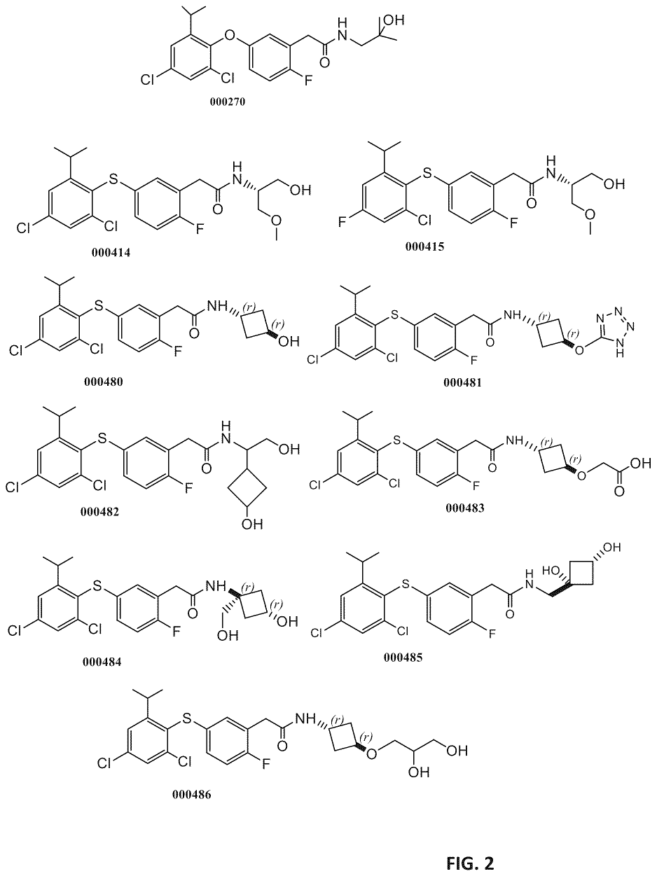

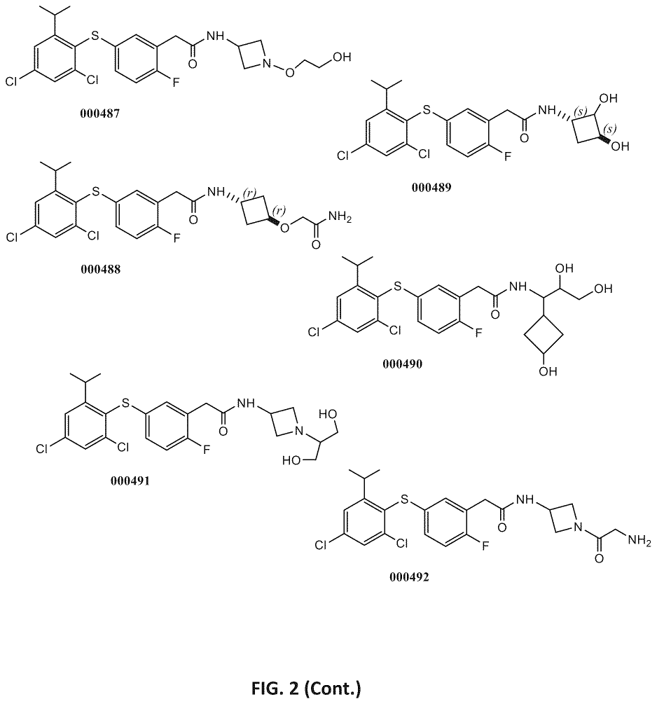

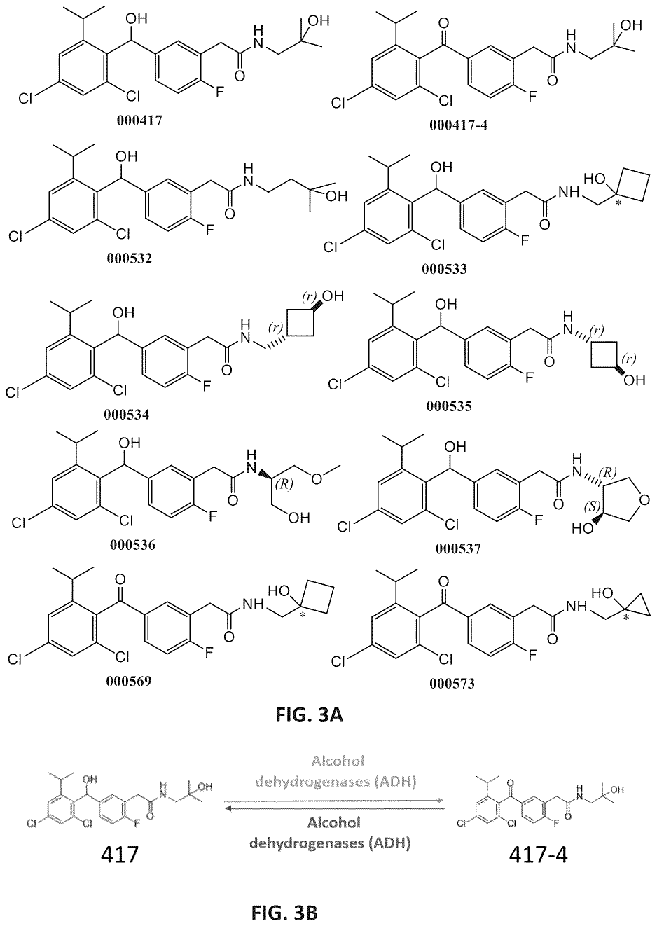

These chemotypes include modifications of the bridge as follows:
Chemotype 1: replacing the Ring A-NH—aniline moiety by a rigid indole moiety; see, FIG. 1 for representative structures
Chemotype 2: replacing the amine bridge by —S— or —O—; see, FIG. 2 for representative structures
Chemotype 3: replacing the amine bridge by —CH(OH)—; see, FIGS. 3A-B for representative structures
Chemotype 4: replacing the amine bridge by —C(═O)—; see, FIGS. 3A-B for representative structures

In addition, amide isosteres have been considered as replacing the amide moiety within the side chain portion. Exemplary such isosteres include an oxetane structure (see, for example, FIG. 1; compound 000526) and triazole structure (see, for example, FIG. 1; compound 000527).

Further in addition, compounds featuring a heteroaryl (e.g., pyridine) as Ring B were considered. Exemplary such compounds of chemotype 1 include compounds 000661, 000662, 000663 and 000649 (see, FIG. 1).

The biological and physicochemical properties of representative compounds of each chemotype are presented in Table 1 below.

It is to be noted that the chemotype 3 compounds (featuring a dihydroxy structure) and corresponding chemotype 4 compounds (featuring a ketone bridge) were found to be conceptually interesting, since the dihydroxy chemotype 3 can be oxidized into its corresponding ketone structure by the liver enzyme Alcohol dehydrogenase (ADH), as shown in FIG. 3B. As the enzymatic reaction is reversible, the two structures could be possibly generated according to the body's needs. Under this scenario, dihydroxy chemotype 3 is expected to be active mainly against Kv7.2/7.3 (EC50=5.6 μM) whereas its correlated in-situ oxidized ketone structure chemotype 4 is expected to be active mainly against TRPV1 (IC₅₀=8 μM).

TABLE 1

| | TRPV1 | Kv7.2/7.3 | | | | | |
|---|---|---|---|---|---|---|---|
| | IC50 | IC50 | HLM Clint | Kinetic | | hERG IC50 | CNS |
| Compound | [μM] | [μM] | [μl/min/mg] | Sol. | LogD | [μM] | PMO |
| 421-6 | 2.1 | 1.1 | 55.5 | 3.4 | 5.47 | >>2.5 (0%) | 3.1 |
| 414 | 1.1 | 1.8 | 686 | | | >>2.5 (0%) | 2.6 |
| 415 | 7.7 | 2.2 | 839 | 26.7 | 4.25 | >>23 (0%) | |
| 417 | 26.6 | 5.6 | 89.1 | | | >>2.5 (0%) | 2.8 |
| 541 | 3.2 | 4.7 | 772.2 | 2 | 4.78 | | |
| 552 | 1.0 | 12 | 27.2 | 5 | 4.77 | | |
| 627 | 1.9 | 0.54 | <9.6 | 6 | 4.85 | >>2.5 (0%) | 3.89 |
| 533 | 21.2 | 1.5 | 657.9 | 41 | 4.36 | >>50 (0%) | |
| racemic | | | | | | | |
| 417-4 | 8 | 13.4 | 91.84 | 66 | 4.08 | >>35 (0%) | |
| 569 | 10 | 3 | 120 | 27 | 4.47 | | |
| 573 | 3 | 7 | 441 | 24 | 4.34 | | |
| 649 | 3.3 | 5.2 | 27.3 | 10 | 4.19 | | |
| 661 | 19.3 | 1.1 | 72 | 40 | 3.55 | >>16.6 (0%) | 4.0 |
| 662 | agonist | 8.0 | 38.1 | 6 | 3.49 | | |
| 663 | 2.2 | 5.9 | 53.6 | 26 | 3.52 | | |

### Example 2

### Computational Modeling

Molecular docking and quantitative Structure Activity Relationship modeling (qSAR) represent the major computational tools employed in computational chemistry. Molecular docking applies the target protein's 3D structure to locate various molecules in a preferred active conformation inside a chosen virtual binding pocket (i.e., Structure Based Drugs Design). qSAR applies various physicochemical as well as 2D and 3D structural elements in a chosen set of molecules to predict measured parameters of compounds (see, Sharma, S., Recent trends in QSAR in modelling of drug-protein and protein-protein interactions. Comb. Chem. High Throughput Screen. 2020).

### Molecular Docking:

To perform docking experiments, homology model of the human TRPV1 was generated. For that purpose, the following steps were performed:
1. Building multiple homology models: one using 5IRX (Prati, F. Stem Cell Translational medicine, 2017; 6:369-381), ligand-bound structure, and 3J5P (SA CAI, et al. Stem Cell Translational medicine, 2017; 6:369-381), the apo structure (“closed conformation”).
2. Analyzing the pockets on both and comparing to mutation data.
3. Docking identified lead compounds to both homology models.

A homology model which is based on the published 3J5P model (SA CAI et al. 2017 supra) was thereby generated (not shown). This model showed additional plausible binding modes and was consistent with the rat/human difference at residue 547.

Homology model of the human Kv7.2/7.3 was based on the open state of Kv7.1 (Peretz et al., Proceedings of the National Academy of Sciences August 2010, 107 (35) 15637-15642).

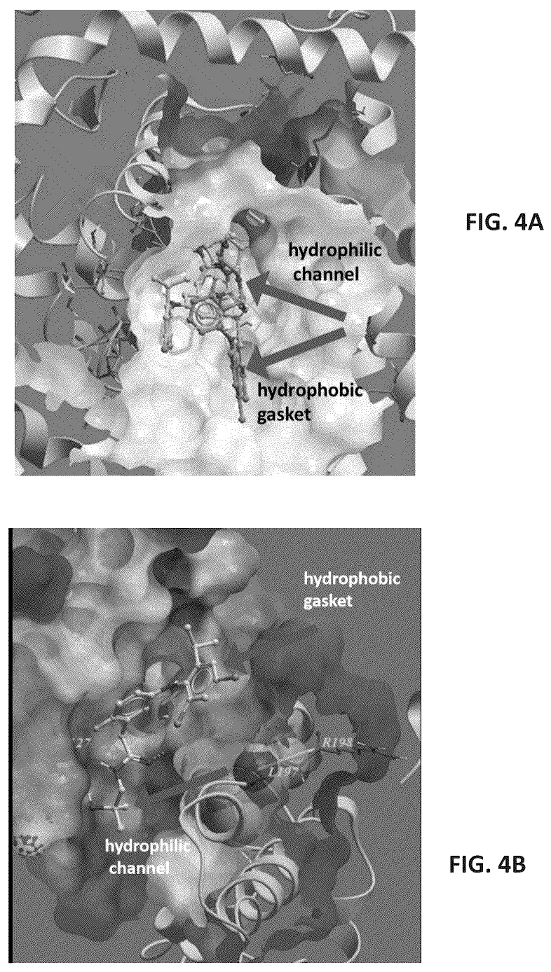

FIG. 4A presents the docking of NH91 (000091; bright yellow), of the known TRPV1 inhibitor resiniferatoxin (RTX), and of Compound 228 (a compound as described, for example, in WO 2004/035037) inside the vanilloid pocket of the respective homology model. FIG. 4B presents the docking of NH91 inside the vanilloid pocket of the homology model of the Kv7.2/7.3.

As can be seen, the hydrophobic gaskets and hydrophilic channels are shared structural motifs of both proteins, which allow the design of dual Kv7.2/7.3 and TRPV1 modulators.

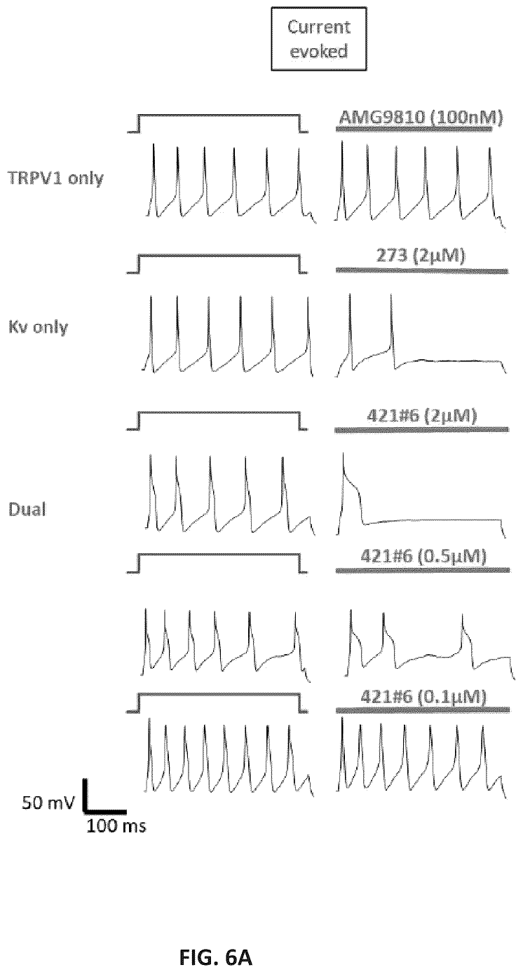

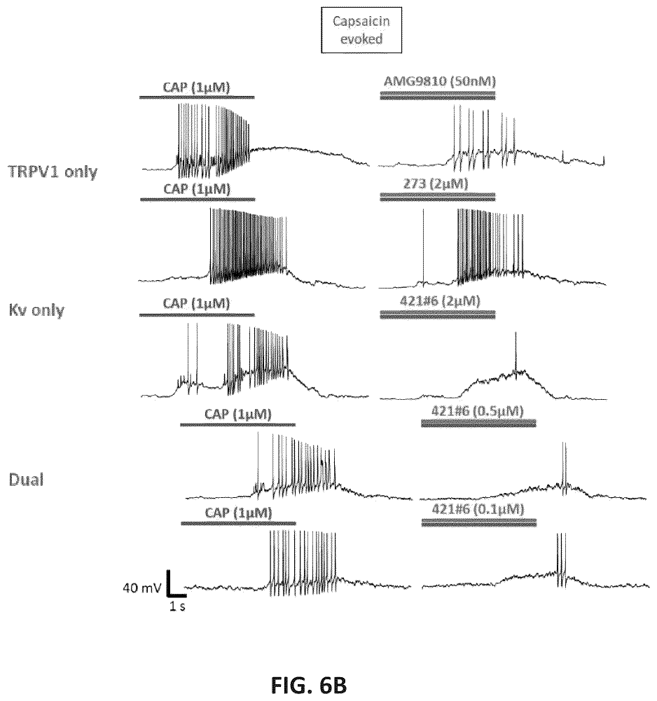

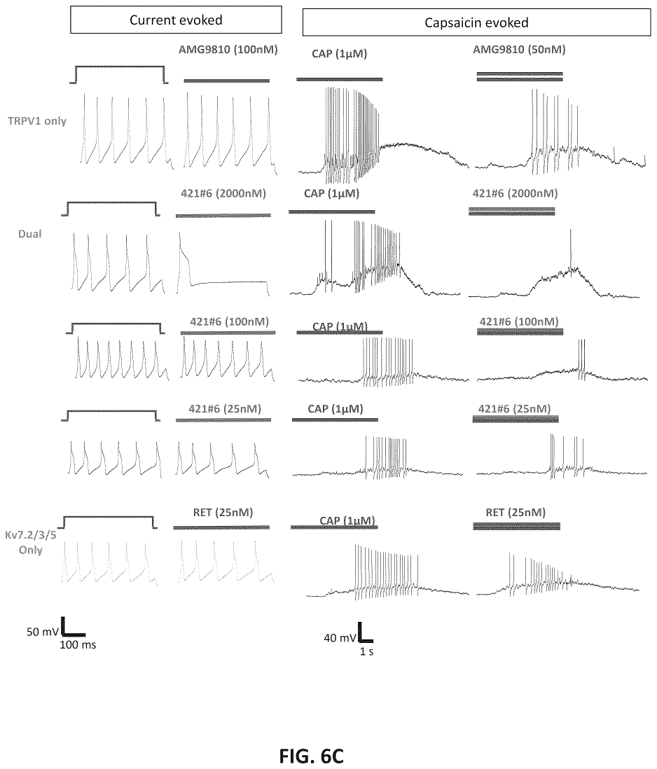

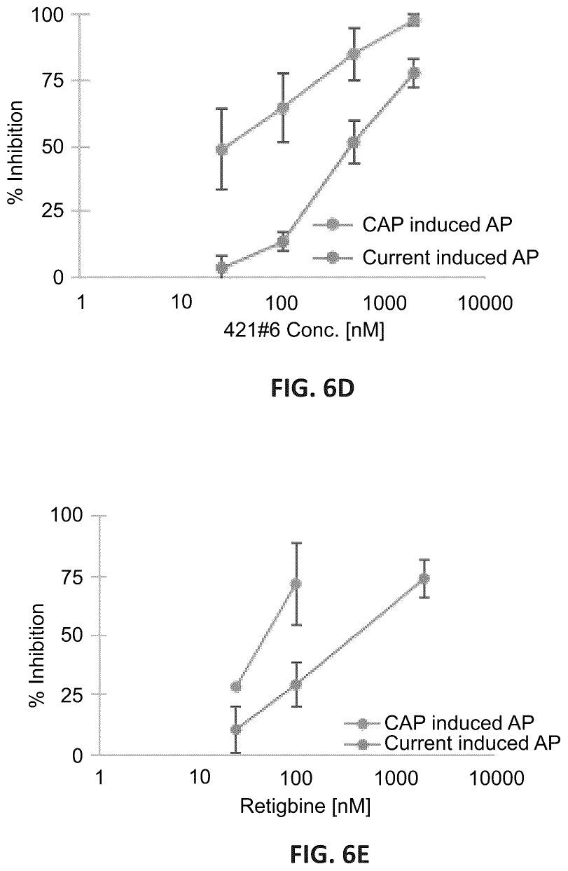

Based on the three-dimensional structure of the human TRPV1 and the uncovered preferred binding poses for the tested compounds (FIG. 6A), the following was deduced:
=The newly designed compounds have high probability of binding and interacting with the TRPV1's vanilloid pocket (i.e., capsaicin binding pocket);
=The ‘A’ and ‘B’ rings preferably interact with a hydrophobic surface area (i.e., hydrophobic gasket);
=The polar side chain is preferably located into a hydrophilic channel.

Based on the three-dimensional structure of the human Kv7.2/7.3 and the uncovered preferred binding poses for the tested compounds (FIG. 4B), the following was deduced:
=The terminal hydroxyl (—OH) as well as the hydrophobic A ring preferably interact with a hydrophilic channel and a hydrophobic gasket, respectively;
=The aniline bridge results in a non-optimized rigid structure, pointing towards modifications at this pharmacophoric site as a promising pathway for improved activity against Kv7.2/7.3.
   Quantitative Structure Activity Relationship Modeling (qSAR):

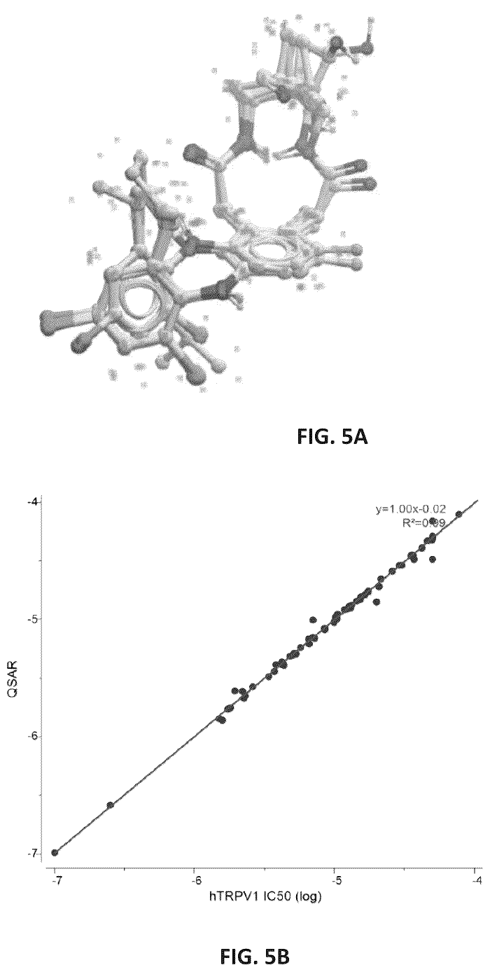

For creating such models, some of the hit compounds described herein were superimposed one on top of the other in their calculated 3D stable conformation and a pharmacophoric representation was created, as shown in FIG. 5A. Based on the pharmacophoric representation, various learning models were created, which allowed correlating all the IC50/EC50 values (generated for TRPV1 or Kv7.2/7.3, respectively) with their structural elements, as shown in FIG. 5B for TRPV1.

Exemplary data of representative TRPV1 qSAR modelling outcome is presented in Table 2.

TABLE 2

| | qSAR | Experimental |
|---|---|---|
| Compound | IC50 | IC50 |
| No. | [μm] | [μm] |
| 414 | 6.3 | 1.1 |
| 415 | 7.8 | 7.8 |
| 480 | 2.1 | 1.4 |
| 422 | 8.3 | 2.2 |
| 405 | 88 | 125 |

The capability of a representative TRPV1 qSAR model to highly correlate between the experimental IC50 values and qSAR predictions is clearly shown.

A computational infrastructure was therefore generated, consisting of both homology models and docking capabilities as well as qSAR learning models.

### Example 3

### Efficacy Studies

### Effect on Rat DRG Neurons:

The inhibitory potency of the newly designed compounds was studied on neuronal excitability in an experimental system of high physiological relevance, primary neonatal rat DRG neurons (Sprague Dawley neonatal; P2,3; R-DRG-505, Lonza). These cryopreserved DRG cells are prepared from freshly isolated and dissociated spinal cord dorsal root ganglia and comprise a normal distribution of neurons and glia (schwann cells).

The inhibitory potency was tested both on current-induced and Capsaicin-induced neuronal activity. The obtained data is shown in FIGS. 6A-E.

As can be seen in FIG. 6A, with current stimulation, the inhibitory potency of the tested compounds is mediated and identified through their effects on the voltage-activated effectors, such as the Kv7.2/3 target. As can be seen in FIG. 6B, Capsaicin evoked responses identify the contribution of both the Capsaicin-gated TRPV1 target, which depolarize the membrane upon activation, and the consequently activated voltage-gated Kv7.2/3 target downstream. Thus, with both targets being activated, the add-on effect of dual targeting of TRPV1 and Kv7.2/3 can be compared with only single Kv7.2/3 targeting compound such as Retigabine that have similar EC50 for Kv7.2/3 as 421-6.

More specifically, AMG9810 inhibitory potency was tested first, both on current and Capsaicin evoked neuronal activation. Capsaicin evoked firing was reduced by about 50% with AMG9810 (50 nM) application. On the contrary and as expected, the current evoked response remains unchanged since AMG9810 does not target any known voltage dependent component of the action potentials firing.

Next, compound 273, showing Kv-only targeting (Kv7.2/3 EC50=1.3 μM), was examined (2 μM) and showed a partial reduction of current evoked neuronal activation. Capsaicin evoked neuronal activation, which reads-out the combination of both targets, also has showed a similar reduction in its AP response, supporting the effect of Kv activation as a general mechanism of reduction in action potential firing regardless of the activation mechanism (current or Capsaicin induction).

Then, compound 421-6, which exhibits TRPV1 and Kv7.2/3 dual targeting, was examined (Kv7.2/3 EC50=2.1 μM; TRPV1 IC50=1.8 μM). Applying 2 μM of 421-6, a dose which aligns with its Kv7.2/3 EC50 and TRPV1 IC50, dramatically reduced the AP response to both current and Capsaicin activation, showing inhibitions of 77.7±5.6% (N=3) and 97.9±2.1% (N=2) respectively. The pronounced inhibition of neuronal voltage dependent AP activity was higher than the 50% inhibition of Kv7.2/3 activity observed measuring the K influx through these channels expressed in heterologous system. This might reflect changes evolving from aspects resembling the different nature of these two readouts, given that one directly measures the gradually accumulated ion conducted through the gated ion-channel target and the other reads the physiological relevant outcome which is a threshold all-or-none phenomenon regulated also by the target. Possible different target interactions and compositions (e.g. different Kv subunits stoichiometry and relative expression) in the endogenous versus the heterologous system may also explain these findings.

Since neuronal excitability is a physiological predictive readout of pain propagation and in light by the apparent high potency of compound 421-6 in reducing neuronal firing, its IC50 value inhibiting firing responses in DRGs neurons was tested. To this end, the activity of compound 421-6 was tested at various nM concentrations.

The obtained data, along with comparative data obtained for retigabine (RET), is shown in FIGS. 6A-E. Diluting 421-6 significantly reduced its inhibition of current induced AP responses, reaching its IC50 around 500 nM (51.2±8.4% N=3), retaining a significantly higher inhibition of the capsaicin induced AP response, which reflects the impact of dual action on both targets (86.5±8.4%, N=4). Furthermore, 421-6 dilution to a concentration of 100 nM, inhibited current induced AP activity by only 10.1±3.4% N=2, while still preserving a high inhibition potency of capsaicin induced AP response (63.4±17.8%, N=3), emphasizing both the high potency and synergistic activity of this dual-targeting compound.

Compound 421-6 dual targeting compound displays a <100 nM inhibition of capsaicin induced neuronal firing. This high inhibition potency, significantly above the sum of inhibitions contributed by each target alone, indicates the synergistic effect of 421-6. This synergism evolves (1) the high inhibition potency for such a compound (2) a superior specificity that emerges from a higher activity occurs only where both targets are co-expressed and their signaling pathways are crossing, which is unique to the nociceptive sensory neurons. Such a synergism might occur when a second messenger of one target is a modulator of the other one. For example, TRPV1 inhibition by the compounds of the present embodiments inhibits the Ca2+ influx through this channel which consequently might relief calcium mediated inhibition of Kv7.2/3 channel, to synergistically activate the M-current together with the direct Kv7.2/3 activation by the same compound.

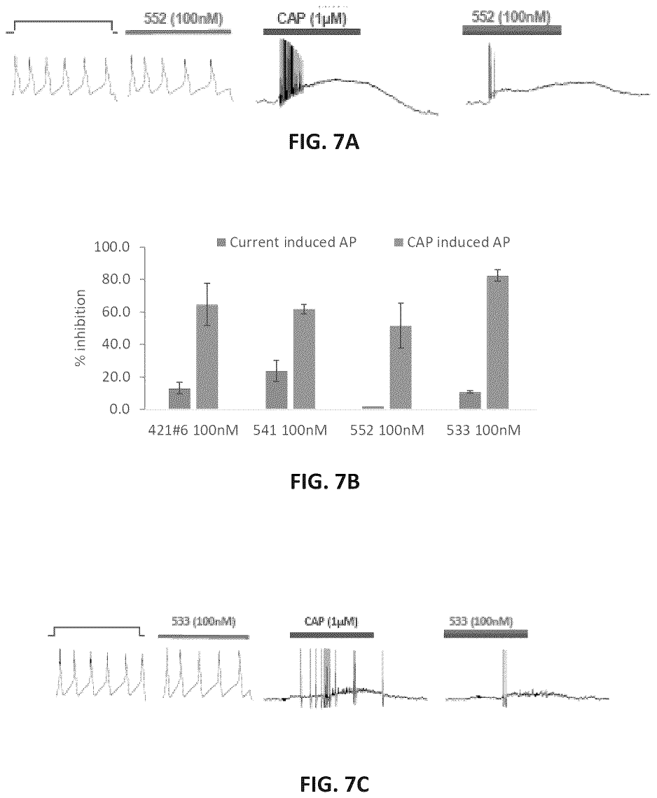

FIGS. 7A-C present data obtained in these assays for other representative compounds according to the present embodiments, denoted Compounds 552 and 541 (see, FIG. 1) and 533 (see, FIG. 3B).

### Neuropathic Model in Rat DRGs:

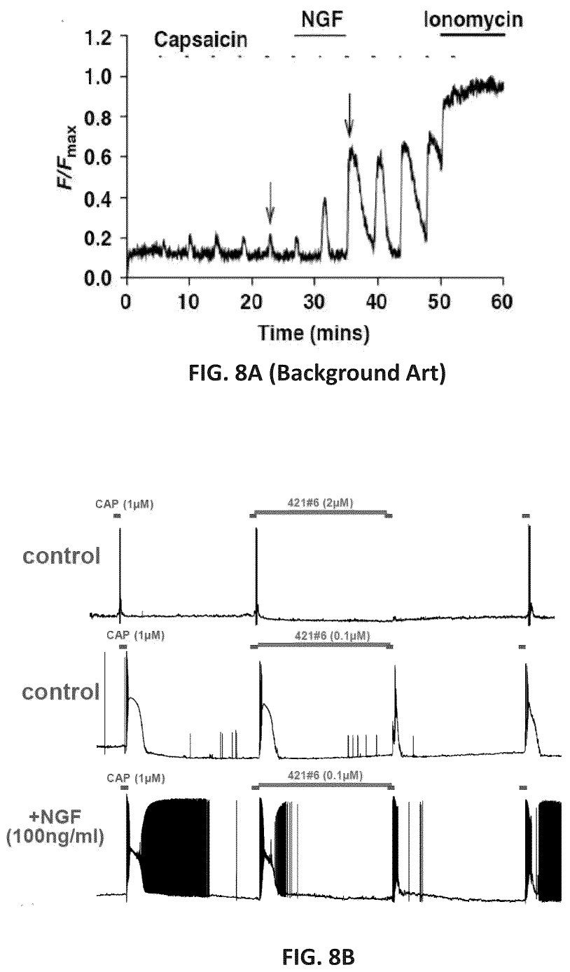

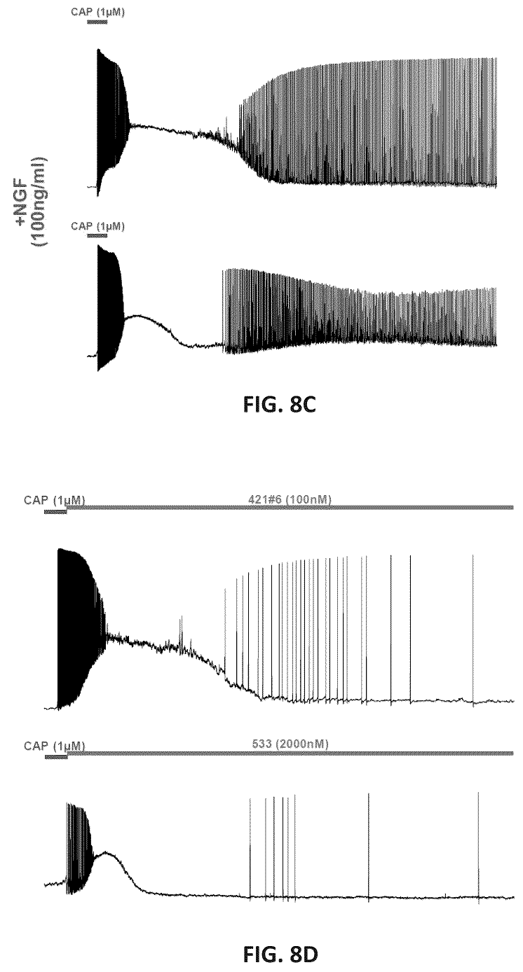

DRGs treated with NGF show tetanic action potential burst in response to CAP application, mimicking neuropathic like response. See, Background Art FIG. 8A. NGF enhances TRPV1 function using Calcium-dependent fluorescence, F, relative to maximal fluorescence, Fmax, as a function of time from a single HEK293 cell stably transfected with TrkA and transiently transfected with hTRPV1. Pulses of capsaicin (100 nM, applied as shown at top) elicited submaximal increases in [Ca]i. Exposure to NGF (100 ng/mL, see top) enhanced the capsaicin induced calcium increase. Arrows show responses used for calculation of sensitization ratio.

As shown in FIGS. 8B-D, compound 421-6 alleviates the capsaicin induced tetanic action potential burst at levels below EC50, supporting the advantage of dual Kv/TRPV1 modulation. As shown in FIG. 8B, the second tetanic burst is modulated as 421-6 reaches steady state concentration in the recording chamber. The effect is maintained following the third application of capsaicin as the concentration decreased with the washout, suggesting a slow off rate kinetics. As can be seen following the fourth capsaicin application, the effect is reversible.

### Effect on Human NPCs:

To study the inhibitory potency of exemplary leading compounds on neuronal excitability in an experimental system that resembles their effect on human pathophysiology, human neural progenitor cells (hNPCs) were differentiated to human sensory neurons and following their electrical maturation, their excitability was examined with or without the tested candidates.

Differentiated human sensory neurons went through electrical maturation, showing polarized resting membrane potential that became more and more negative along maturation, as expected from mature neurons with their increased population of K channels, shifting the resting membrane potential toward the potassium reversal potential.

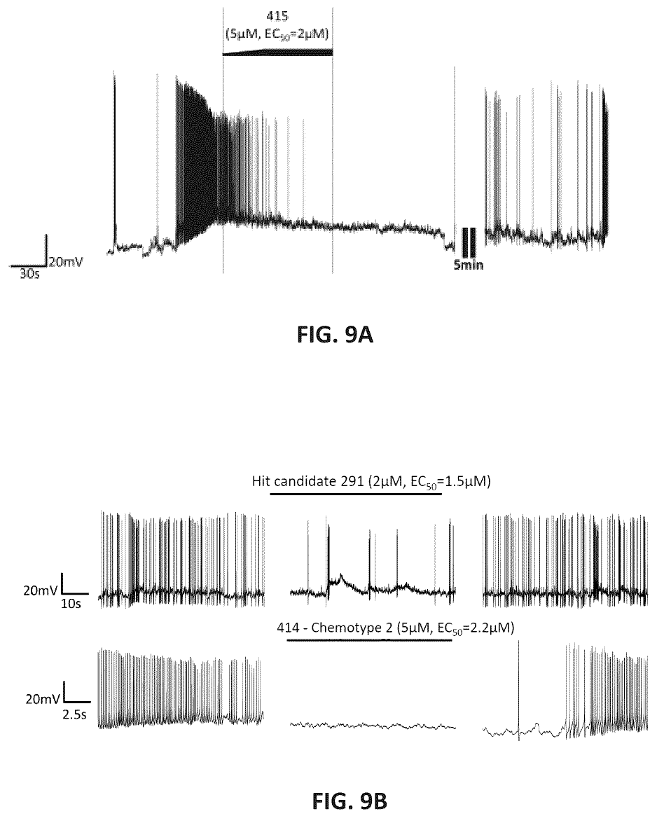

The inhibitory potency of the newly designed compounds as described herein was evaluated based on their ability to reduce the spontaneous neuronal activity. As shown in FIG. 9A, a differentiated human sensory neuron, shows spontaneous activity which was significantly reduced following application of a chemotype 2 compound 415 (5 μM) in a reversible manner. Compound 415 displayed a strong inhibitory potency in human sensory neurons, inhibiting spontaneous firing and the need to approach the targets at a much lower, predicted nM concentrations for effective concentration without potential adverse effects.

FIG. 9B presents the data obtained for a chemotype 2 compound 414, compared to compound 219, which bears an aniline bridge:

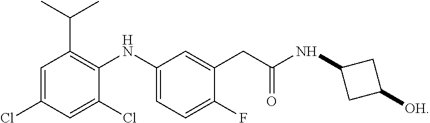

These data show the strong inhibition potency of the newly designed compounds as described herein on the excitability of rat and human sensory neurons.

### Example 4

### Further Insights on the Mechanism of Action

### Effect of Racemic Mixture and Separated Enantiomers:

To test whether different enantiomers of molecules that feature a single chiral carbon exhibit different activity, the compound 533 was selected as a representative example (* denotes a chiral center).

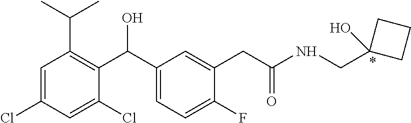 [SMILES: CC(C)c1cc(Cl)cc(Cl)c1C(O)c1ccc(F)c(CC(=O)NCC2(O)CCC2)c1]

The racemic mixture and the two enantiomers, separated using SFC methodology and denoted as 533p1 and 533p2, were tested in efficacy studies as described in Example 3 hereinabove.

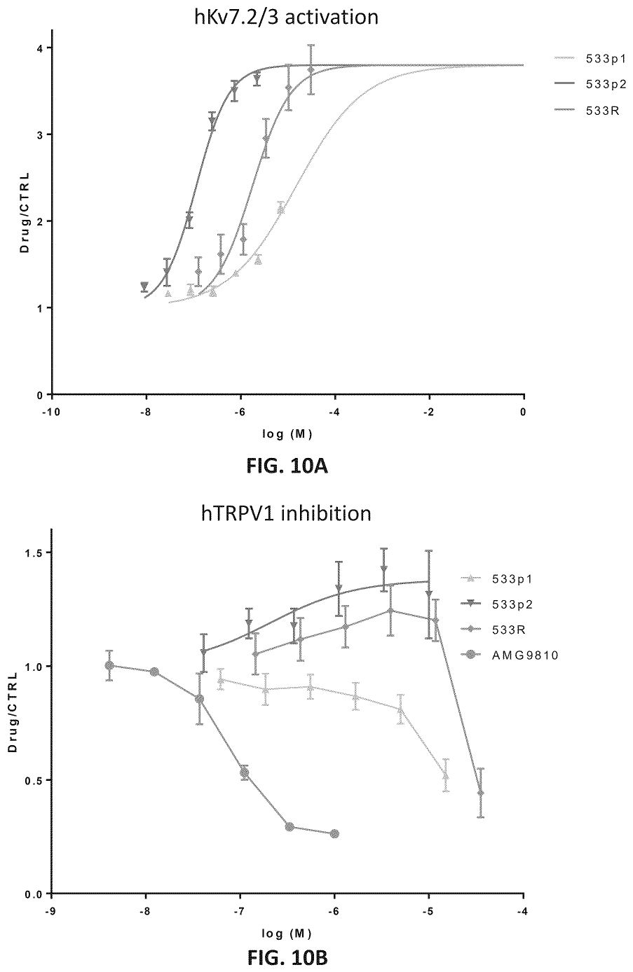

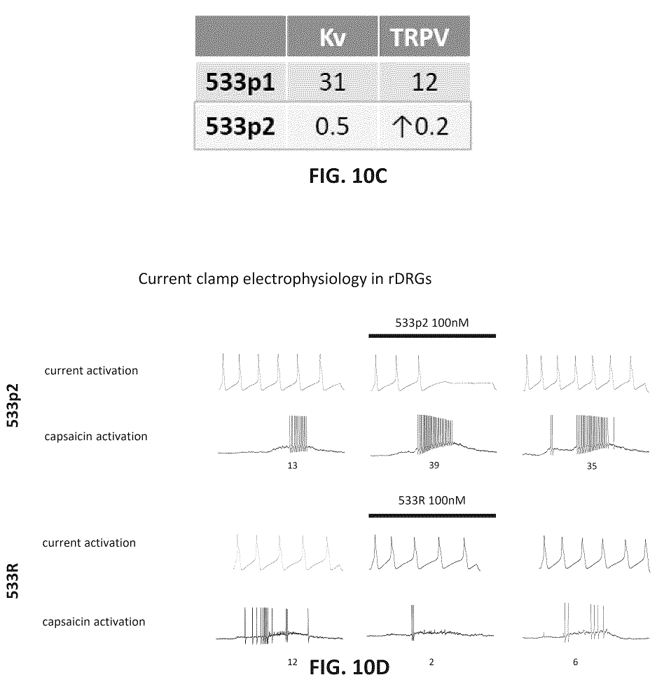

FIGS. 10A-C present the inhibitory potency of the tested compounds and reference compounds on current-induced and Capsaicin-induced neuronal activity, and show that a stereospecific modulation can be seen in 533 compound, such that 533P1 shows primarily activation of Kv7.2/3 and TRPV1 inhibition while 533P2 shows a more potent activation of Kv7.2/3 while activating TRPV1. This is exemplified in FIG. 10D, by current and capsaicin induced AP in rat DRG, in which 533P2 inhibits current induced AP and potentiates capsaicin induced AP while the racemic mixture which combines the effects of the two enantiomers shows reduced current induced AP inhibition while highly potentiating capsaicin induced AP inhibition.

### Dual Cross-Talk Modulation:

Compound 627 (see, FIG. 1 and Table 1) was selected for gaining further insight on the mechanism of action.

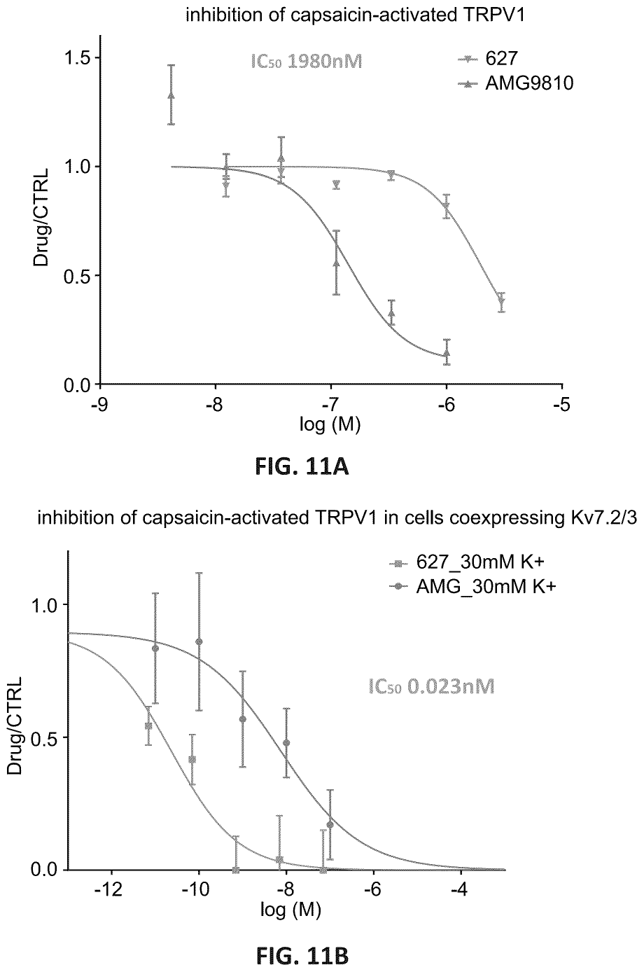

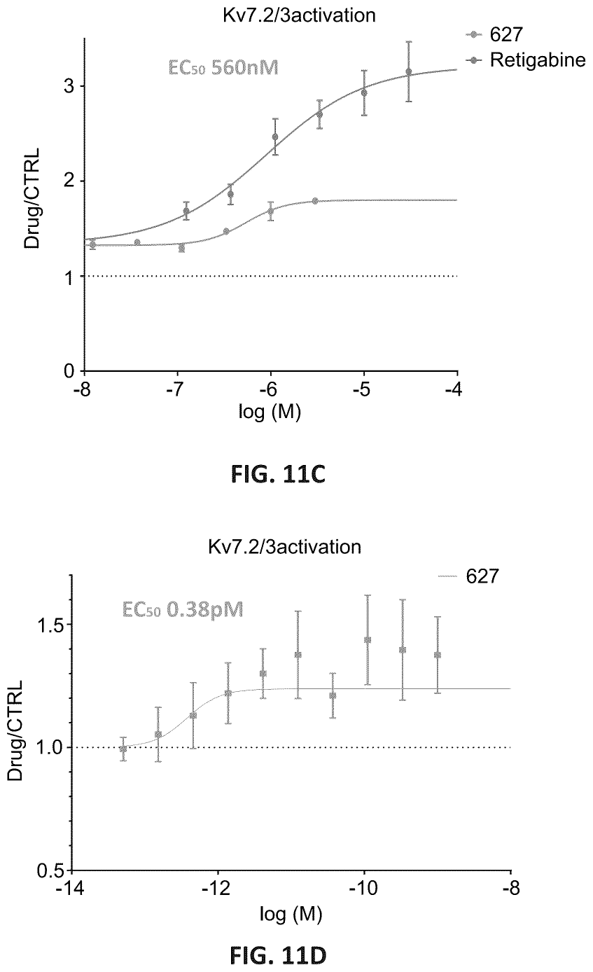

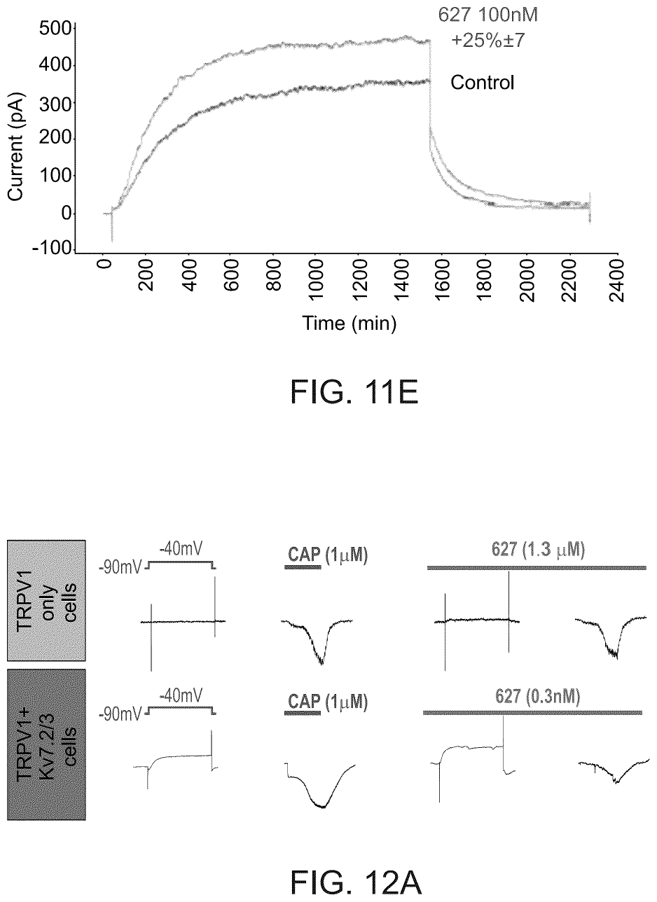

CHO cells expressing hTRPV1 or co-expressing hTRPV1 and hKv7.2/3 were treated with compound 627 (see. FIG. 1), with AMG9810, a known TRPV1 inhibitor used as a positive control, or with retigabine. The obtained data is presented in FIGS. 11A-E.

FIGS. 11A-B present comparative plots showing the TRPV1 inhibition by compound 627 compared to retigabine. It can be seen that when hKv7.2/3 is co-expressed and activated using chemical depolarization, the potency of compound 627 increases significantly, shifting leftward the dose-response curve, significantly stronger than the inhibition potency of AMG9810.

FIGS. 11C-D present plots demonstrating the inhibition of cells co-expressing hKv7.2/3, and showing that compound 627 at low concentration displays a significant hKv7.2/3 activation even at picomolar concentrations (FIG. 11D).

These data show that compound 627 displays a TRPV1 IC50 of 1.9 μM and Kv7.2/3 EC₅₀ of 0.5 μM, when applied to CHO cell-lines solely expressing hKv7.2/3 or hTRPV1, as measured using fluorescent HCS, and displays about 30% Kv7.2/3 activation already at a concentration as low as 10 nM range.

As can be seen in FIG. 11E, activation of hKv7.2/3 activity by compound 627 was confirmed using electrophysiology, measuring whole-cell currents of hKv7.2/3 expressed in CHO cells, clamping membrane potential to −40 mV (1.5 s) (from a holding potential of −90 mV) (N=4). Compound 627 at 100 nM showed Kv7.2/3 current activation by 24.4±10.4%.

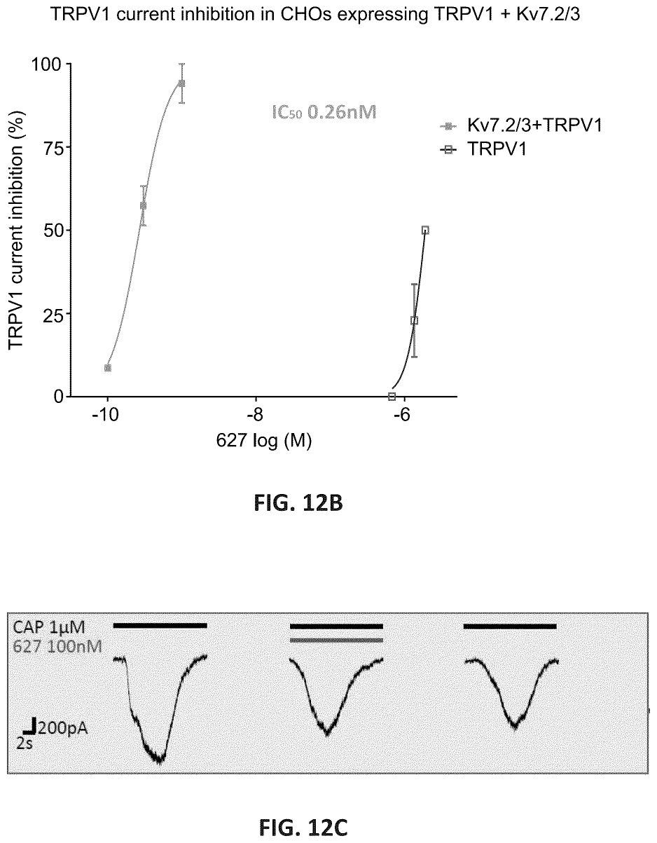

To exemplify the functional coupling of Kv7.2/3 and TRPV1, stably expressing Kv7.2/3 cells were transfected with human TRPV1 and their response to current ramp to −40 mV (Kv activation) and capsaicin (TRPV1 activation) was examined electrophysiologically. The obtained data is shown in FIGS. 12A-C.

FIG. 12A, upper panel, shows the effect of hKv7.2/3 co-expression in gaining potent 627 hTRPV1 inhibition. While hTRPV1 expressing cells show partial TRPV1 inhibition by 1.3 μM of compound 627, when hKv7.2/3 is co-expressed and activated (lower panel), compound 627 gains subnanomolar hTRPV1 inhibition potency.

FIG. 12B presents comparative dose-response plots displaying the hTRPV1 inhibition-potency gained (leftward shift) in the presence of compound 627 when hKv7.2/3 is co-expressed and activated.

FIG. 12C presents exemplary currents response to capsaicin in CHO cells co-expressing hTRPV1 and hKv7.2/3, but without hKv7.2/3 activation, in the presence of compound 627, and show that it displays a lack of hTRPV1 inhibition-potency gain, even at a concentration of 100 nM.

The data presented in FIGS. 12A-C indicate that the potentiation of the TRPV1 inhibitory potency increases by 4 orders of magnitude (from 2 μM to 0.26 nM) and requires Kv7.2/3. In addition, this potentiation of TRPV1 inhibition has a precondition of Kv7.2/3 activation as it is shown that without precondition of current ramp to −40 mV (Kv activation) TRPV1 could not be inhibited even at 100 nM.

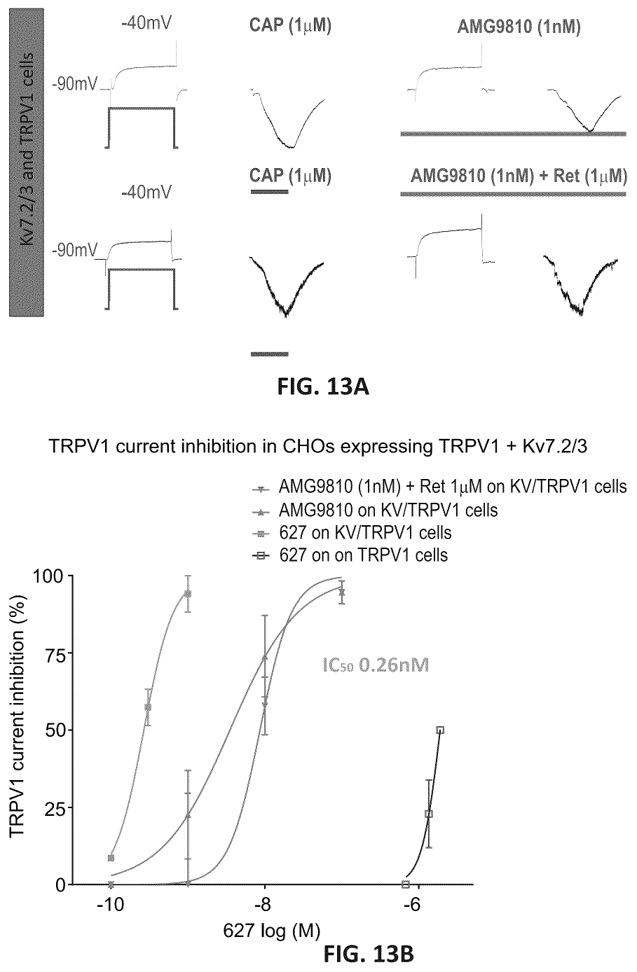

FIG. 13A shows that in the presence of AMG9810, a known TRPV1-inhibitor, failure to gain TRPV1-inhibition potency is observed, even when it is co-applied with the known Kv7.2/3 opener retigabine, in cells co-expressing both hKv7.2/3 and hTRPV1.

FIG. 13B presents comparative dose-response plots demonstrating similar hTRPV1 inhibition-potency of AMG9810 and of AMG9810 and retigabine combination treatment, indicating no synergistic effect therebetween. Contrary, compound 627 exhibits different hTRPV1 inhibition-potency when hKv7.2/3 is co-expressed and activated or when hTRPV1 is expressed alone. These data show that TRPV1 inhibition is uniquely effected by the same compound when Kv7.2/3 is co-expressed and activated.

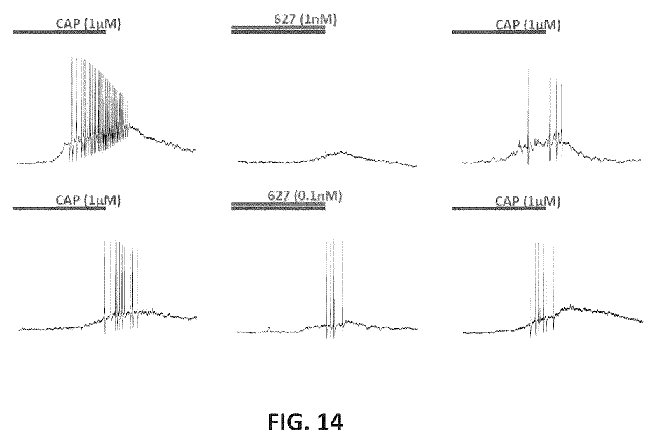

FIG. 14 presents an exemplary rat DRG membrane-potential recording showing action-potentials trains in response to Capsaicin application in the presence of compound 627. As can be seen, application of compound 627 at a concentration of 1 nM completely blocked the Capsaicin-evoked firring (upper panel), and about 50% inhibition of action-potentials firing was observed when compound 627 was applied at a concentration of 0.1 nM (0.04 ng/mL).

### Example 5

### Efficacy Studies on an Exemplary Chemotype 3 Compound

Compound 661 (see, FIG. 1 and Table 1) was selected to further study its hTRPV1 dual activity.

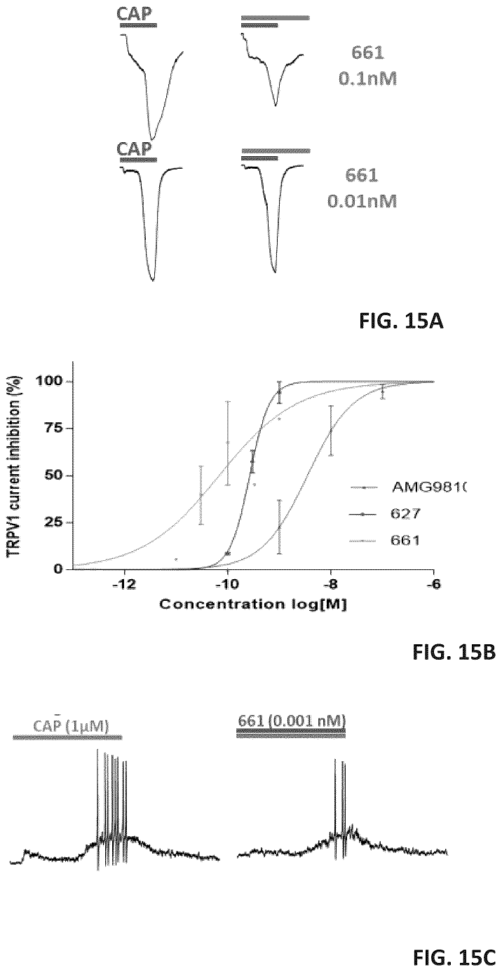

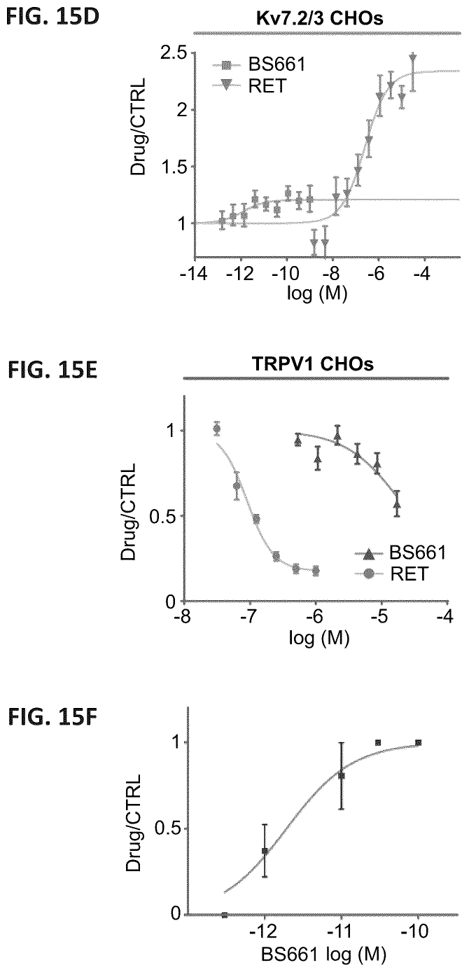

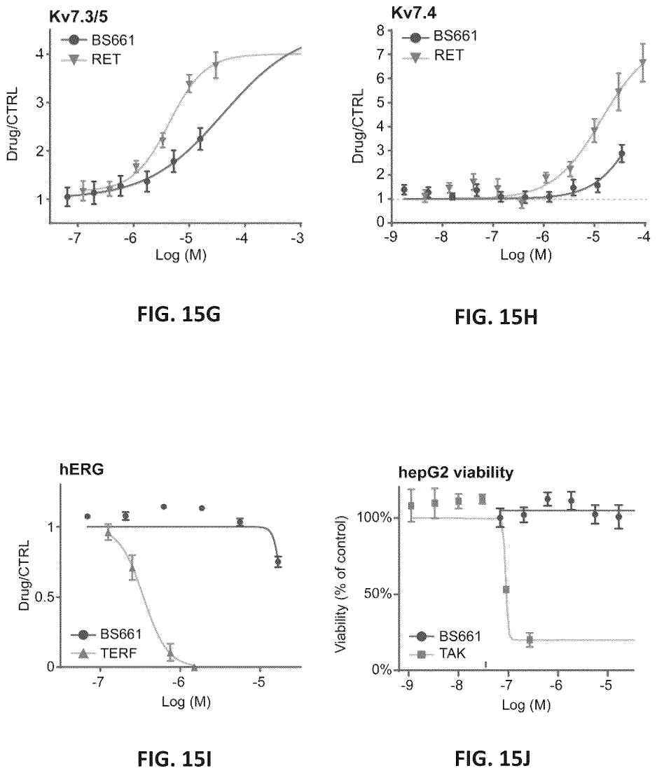

CHO cells co-expressing hKv7.2/3 and hTRPV1 following hKv7.2/3 activation, were treated with compound 661 (see FIG. 1). The obtained data are presented in FIGS. 15A-C.

FIG. 15A shows that CHO cells co-expressing hKv7.2/3 and hTRPV1 following hKv7.2/3 activation, are inhibited by compound 661 at a concentration of 0.1 nM, while a concentration of 0.01 nM results in mild inhibition.

FIG. 15B presents dose-response comparative plots displaying the hTRPV1 inhibition potency in CHO co-expressing hKv7.2/3 and hTRPV1 hKv7.2/3 activation, in the presence of compound 661 with 627 (see FIG. 1), and AMG9810, a known TRPV1 inhibitor used as a positive control.

FIG. 15C presents data obtained for rat DRG neuron firing in response to capsaicin application using compound 661 at 0.001 nM, showing about 50% inhibition of action potential firing.

The data presented in FIGS. 15A-C indicate that, similar to compound 627, the potentiation of the TRPV1 inhibition potency of compound 661 increases substantially in the presence Kv7.2/3 co-expression and activation. The potentiation of the hTRPV1 inhibitory potency of compound 661 is over 30-fold higher in physiological conditions (rat DRGs) from 0.1 nM for 627 to 0.003 nM for 661.

Further, the high potency of compound 661 against CAP-mediated TRPV1 channel activation in dual-expressing CHO cells is dramatically greater than that observed in CHO cells expressing only TRPV1 (>200,000-fold) and approximately 70-fold greater than the compound's intrinsic potency at Kv7.2/3.

As can be seen in FIG. 15C, compound 661 showed improved potency on CAP-driven neuronal-spike discharge in rat DRG neurons, with an IC₅₀=0.002 nM, again supporting the compound's novel profile for TRPV1 inhibition by virtue of its dual ion channel targeting MOA.

FIG. 15D presents comparative plots showing activation of Kv7.2/3 activity, measured using fluorescent assay, by compound 661 compared to retigabine positive control.

FIG. 15E presents comparative plots showing activation of TRPV1 activity, measured using fluorescent assay, by compound 661 compared to AMG9810 positive control.

FIG. 15F presents exemplary capsaicin-induced current in CHO cells co-expressing hTRPV1 and hKv7.2/3 and activated in the presence of compound 661.

As can be seen, compound 661 presents a potent inhibition of TRPV1 activity only in dual-expressing cells, following Kv7.2/3 activation, which is supported by its partial yet potent facilitation of Kv7.2/3 activity.

To address the specificity of compound 661, a wide functional screening panel for compound 661 against 78 targets was performed, and the obtained data is presented in Table 3 below, showing only 9 targets that were modulated by compound 661, all of them with an IC₅₀ above 1400 nM. Based on the IC₅₀ of 0.07 nM of compound 661 for CAP-activated current in Kv7.2/3-TRPV1 co-expressing CHO cells, this results in a 20,000-fold separation in potency vs. this panel of targets. In addition, based on efficacious exposures in vivo, the separation from other targets is still greater than 1000-fold, further supporting the specificity of the compound's MOA. As potency for Kv7.2/3 activation (EC₅₀=0.001 nM; see, FIG. 15D) is greater than that of the dual activity (IC₅₀=0.07 nM), the separation from the off-target panel based on Kv7.2/3 potency is even larger.

TABLE 3

| Target | Compound 661 |
|---|---|
| TRPV1-Kv7.2/3 dual | 0.07 nM (on-target) |
| kappa opioid receptors | 7 μM (PAM EC₅₀) |
| adrenergic α2A | 6 μM |
| adrenergic β2 | 4 μM |
| muscarinic m2 receptor | 5 μM |
| cannabinoid 1 receptor | 1.4 μM |
| dopamine D2 | 10 μM |
| 5-HT1B | 10 μM |
| opioid delta receptor | 10 μM |
| opioid mu receptor | 6 μM |
| No activity up to | hERG, Kv7.1, adrenergic β1, dopamine D1, |
| solubility limit | histamine H2, 5-HT3, androgen receptor, |
| | Cav2.1, Nav1.5, NMDA, nAChR(a4/b2), |
| | GABAA, several kinase targets, several |
| | nuclear hormone receptors, several non- |
| | kinase enzymes (COX-1/2), several |
| | transporters (dopamine, NE) |

FIG. 15G presents comparative plots showing activation of hKv7.3/5 activity, measured using fluorescent assay, by compound 661 compared to retigabine positive control.

FIG. 15H presents comparative plots showing activation of hKv7.4 activity, measured using fluorescent assay, by compound 661 compared to retigabine positive control.

FIG. 15I presents comparative plots showing activation of hERG activity, measured using fluorescent assay, by compound 661 compared to retigabine positive control.

As can be seen in FIGS. 15G-I, profiling compound 661 against other Ky family channels, including Kv7.3/5, Kv7.4, Kv7.1 and hERG (Kv1 1.1) showed no measurable activity at any concentration tested, representing a very large separation based on comparison of the inhibition of CAP-induced current (EC₅₀/IC₅₀>20,000 nM).

FIG. 15J presents comparative plots showing activation of hepG2 activity, measured using fluorescent assay, by compound 661 compared to retigabine positive control.

As can be seen in FIG. 15J, compound 661 showed no evidence of toxicity up to the compound's limit of solubility.

### In-Vivo Analysis of Compound 661:

The in vivo efficacy and pharmacokinetics (PK) profile of compound 661 was evaluated as described hereinabove.

Groups of Sprague Dawley (SD) rats were subjected to compound 661, and the pharmacokinetic exposure and efficacy of compound 661 was measured.

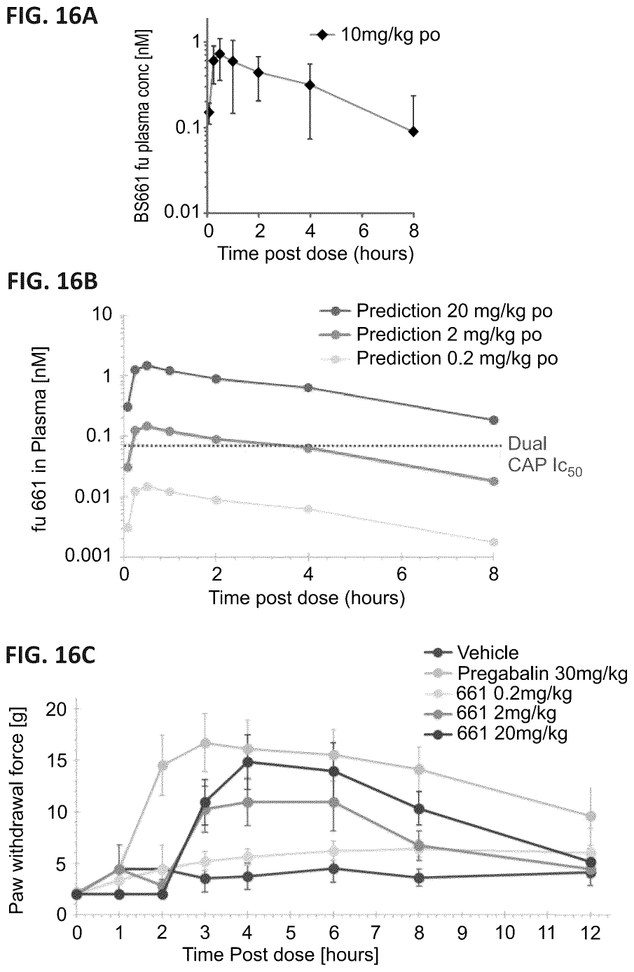

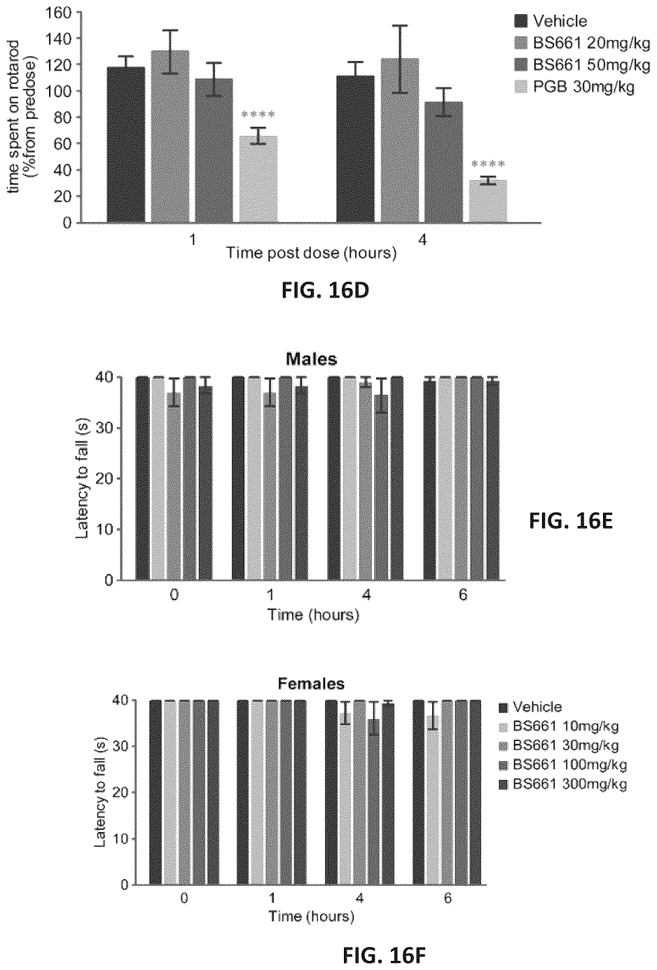

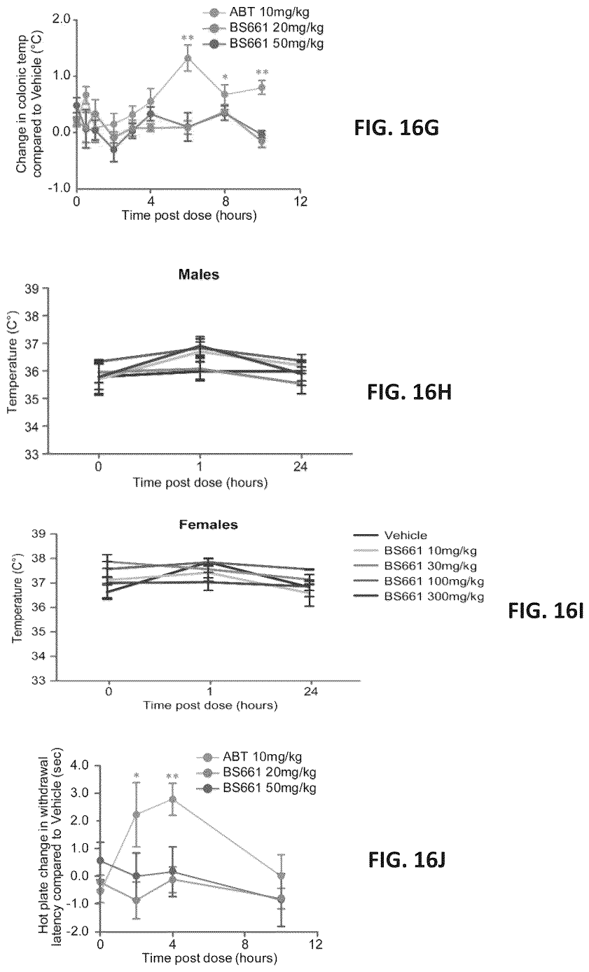

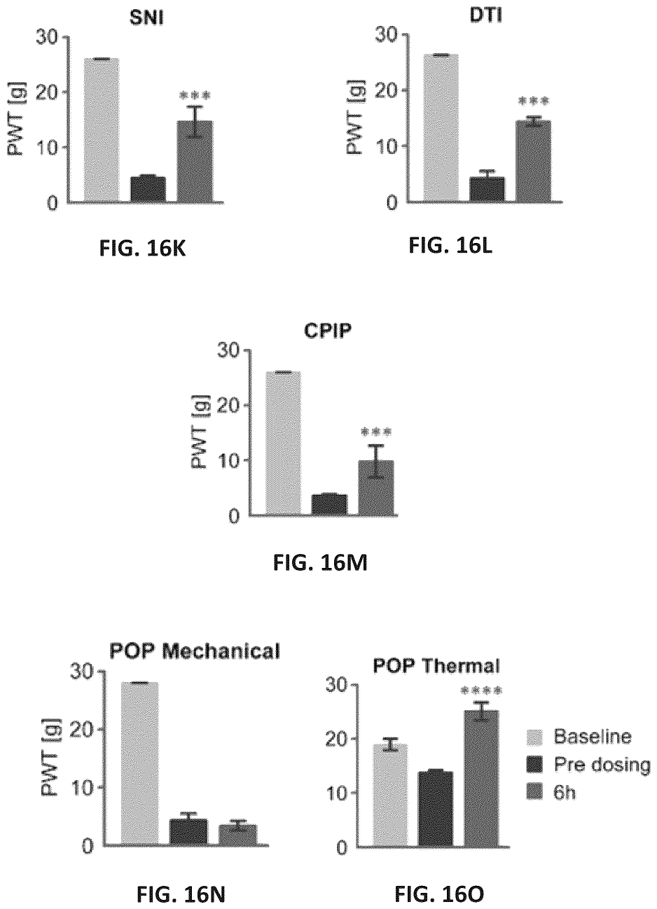

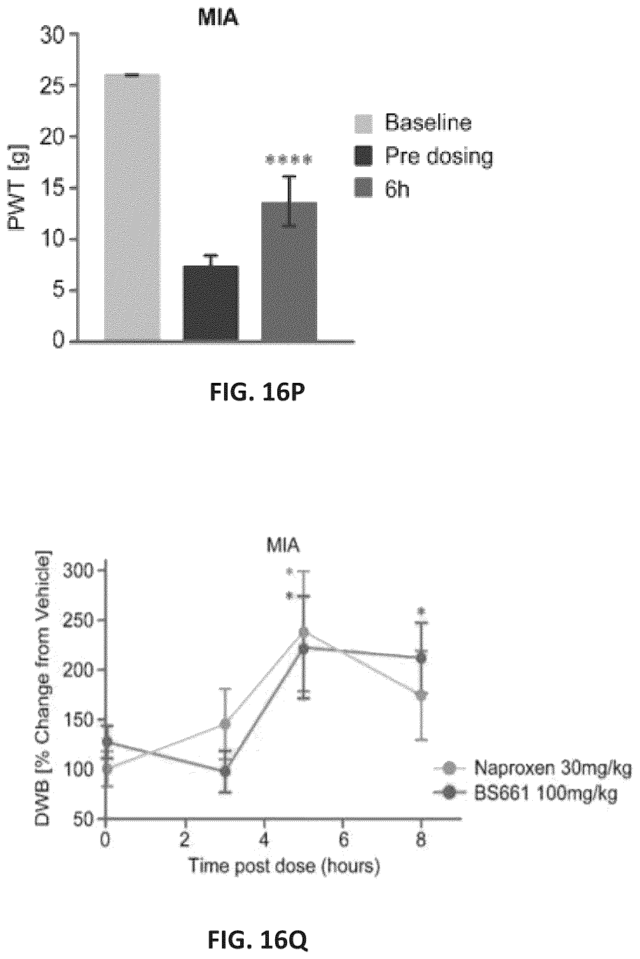

FIG. 16A presents the rat PK exposure of compound 661 following oral delivery of 10 mg/kg. The free plasma exposure across 8 hours (fu plasma C8 hour=0.09 nM) post-dose represent a 30-fold multiple of the IC₅₀ in the CAP-induced neuronal-spike discharge assay (IC₅₀<0.003 nM, rat DRG neurons; FIG. 16A compared to FIG. 15C).

FIG. 16B presents the exposure of compound 661 in Sprague-Dawley rats following oral delivery at 10 mg/kg followed by multiplications to predict 0.2, 2 and 20 mg/kg. The graph indicates that compound 661 exposure is predicted at all doses up to 8 hours after dosing. The plasma exposures in contrast to the dashed red line, representing the IC₅₀ value of compound 661 in the dual action activity assay, indicates that efficacious levels for compound 661 are expected from a 2 mg/kg dose.

Bioanalytical measurement of exposure in the DRG was found to be 13.6% of the plasma free fraction exposure at 8-hour post-dose (0.012 nM). Thus, dose prediction analysis suggested that an oral dose of 2 mg/kg should be sufficient to generate an efficacious exposure in DRGs up to 4 hour post-dose (predicted fu in DRG 0.008 nM), after which exposure would decline below the in vitro IC₅₀ value of 0.003 nM at 8 hour pose dose (predicted fu in RDG 0.0024 nM).

Accordingly, the efficacy profile of compound 661 at 0.2, 2 and 20 mg/kg in the SNI model was tested.

FIG. 16C presents the efficacy profile of compound 661 in the Spared Nerve Injury (SNI) model at doses of 0.2, 2 and 20 mg/kg, compared to pregabalin administered at a dose of (30 mg/kg), and demonstrates a clear dose-response relationship for compound 661, with increasing efficacy, represented by the paw withdrawal force, a measure of allodynia, observed at higher doses. The efficacy profile of compound 661 aligns well with the predicted plasma exposure data presented in FIG. 16A, indicating that higher plasma concentrations of compound 661 correspond to improved efficacy in the SNI model. These data validate the in vitro to in vivo translation of the MoA picomolar potency. At 14 days post-nerve transection, PWT decreased from 26 grams pre-injury to 2.0 grams. Pregabalin at 30 mg/kg PO served as a positive control and showed maximal efficacy at 3 hour post-dose (16.7 grams±2.8, 61% reversal, p<0.001) with significant efficacy maintained up to 8 hour post-dose (14.1 grams±2.2, 50% reversal, p<0.001). Compound 661 at 2 and 20 mg/kg PO showed a dose-proportional reversal of PWT that was maximal at 4 hour post-dose and achieved statistical significance at the 20 mg/kg dose (14.8 grams±2.6; 53% reversal, p<0.01) that was comparable to the effect of pregabalin at this time point. The efficacy of compound 661 at 20 mg/kg remained statistically significant up to 8 hour post-dose (p<0.05 vs. vehicle). The 2 mg/kg dose, although not reaching statistical significance, showed an efficacious trend, confirming that doses achieving several multiples of in vitro IC50 will be required to achieve meaningful in vivo efficacy.

To further investigate the therapeutic potential of compound 661, the compound was evaluated in in vivo adverse effect assays of sedation, motor coordination and core body temperature modulation.

FIG. 16D presents a bar graph showing the efficacy profile in the time spent on rotarod (% from pre-dose) at 1 hour and 4 hour post-dosing, of compound 661 following oral delivery of 20 mg/kg and 50 mg/kg, compared to pregabalin (30 mg/kg) and a vehicle control (N=9), mean SEM, ****p<0.0001 vs. vehicle using Student's T-test).

FIG. 16E presents a bar graph showing the efficacy profile in latency to fall (seconds) over 6 hours post-dosing compared to vehicle, of compound 661 following oral delivery of 10 mg/kg, 30 mg/kg, 100 mg/kg and 300 mg/kg in male rats.

FIG. 16F presents a bar graph showing the efficacy profile in latency to fall (seconds) over 6 hours post-dosing compared to vehicle, of compound 661 following oral delivery of 10 mg/kg, 30 mg/kg, 100 mg/kg and 300 mg/kg in female rats.

As can be seen in FIG. 16D, using the accelerating rotarod, it was found that compound 661 at 20 and 50 mg/kg PO did not affect rotarod performance either at Cmax (1 hour post-dose) or Emax (4 hour post-dose). In contrast, pregabalin at 30 mg/kg resulted in a significant reduction in the latency to fall compared to vehicle-treated rats at 1 and 4 hours post-dose (approximately 65% and 30% vs. vehicle-treated rats, p<0.001 and p<0.0001, respectively). Additional external confirmation of this side effect profile was provided, where compound 661 was evaluated at doses up to 300 mg/kg in both male and female rats and revealed no effects on rotarod performance 1-6 hours post-dose, as shown in FIGS. 16E-F. Using the Modified Irwin Functional Observation Battery multi-parameter (autonomic, neuromuscular, sensorimotor, and behavioral) assessment of nervous system function in rodents, compound 661 showed no effects up to 300 mg/kg on male and female rats, suggesting a greater than 10-fold therapeutic window between efficacious doses in pain models vs. side effects (data not shown).

The core body temperature changes following dosing with compound 661 in comparison to the effect of ABT102, a known TRPV1 antagonist, were also assessed.

FIG. 16G presents comparative plots showing the efficacy profile in the change in colonic temperature compared to Vehicle (° C.) over 12 hours post-dosing, of compound 661 following oral delivery of 20 mg/kg and 50 mg/kg, compared to ABT102 (10 mg/kg).

FIGS. 16H-I present comparative plots showing the efficacy profile in Temperature (° C.) measurements over 24 hours post-dosing compared to vehicle, of compound 661 following oral delivery of 10 mg/kg, 30 mg/kg, 100 mg/kg and 300 mg/kg in male (FIG. 16H) and female (FIG. 161) rats.

As can be seen in FIG. 16G, ABT102 significantly increased the core body temperature of naive rats 6-10 hours post-dose by 1.3-0.8° C., respectively, compared to vehicle-treated rats (p<0.01). In contrast, compound 661 at 20 and 50 mg/kg PO showed no significant change in core body temperature vs. vehicle-treated rats.

In addition, at doses up to 300 mg/kg, compound 661 showed no effects on core body temperature between 1-24 hours post-dose, as can be seen in FIGS. 16H-I.

TRPV1 antagonist compounds also have been shown to increase thermal heat thresholds. ABT102, for example, was found clinically to increase heat pain threshold and reduce painfulness of suprathreshold oral/cutaneous heat (Rowbotham M C et al., Pain 2011; 152:1192-1200).

To address the potential of compound 661 to modulate basal thermal sensation, naive rats were assessed for escape latency to noxious heat using the hot plate assay (53° C.). The obtained data is presented in FIG. 16J, as comparative plots showing the efficacy profile in the paw hot plate change in withdrawal latency compared to Vehicle (seconds) over 12 hours post-dosing, of compound 661 following oral delivery of 20 mg/kg and 50 mg/kg, compared to ABT102 (10 mg/kg).

As can be seen in FIG. 16J, ABT102 at a PO dose of 10 mg/kg showed a statistically significant increase in hot plate escape latency measured at 2 and 4 hours post-dose vs. vehicle-treated rats (p-value<0.05). In contrast, compound 661 at 20 and 50 mg/kg PO had no significant effect on escape latency vs. vehicle control rats 2-10 hours post-dose.

Taken together, compound 661 demonstrated a profile of in vivo pain model efficacy combined with a wide separation from Kv7.2/3 and TRPV1 ion channel-specific side effects typically seen with compounds that selectively target these receptors individually.

Table 4 below summarizes the data accumulated for compound 661, presented in comparison to compounds 421 and 627, showing its superior performance.

As can be seen in Table 4, compared with compound 627, compound 661 showed improved solubility and clearance. In CHO cells expressing TRPV1 or Kv7.2/3 alone, compound 661, like compound 627, showed highly potent activation of Kv7.2/3 but lower maximal activation compared to retigabine, (EC₅₀=0.001 nM; maximal opening 21%, 220% vs. control, respectively; see, FIG. 15D). In addition, compound 661 showed 10-fold weaker intrinsic TRPV1 inhibition compared to either compounds 421 or 627 (IC₅₀=19130 nM, see, Table 4).

TABLE 4

| | | BS421 | BS627 | BS661 |
|---|---|---|---|---|
| On | CAP hTRPVI IC₅₀ HCS (nM) | 1800 | 1900 | 19000 |
| target | hKv7.2/3 EC₅₀ HCS (nM) | 824 | 0.001 | 0.001 |
| | Rat DRGs, CAP IC₅₀ (nM) | 25 | 0.1 | 0.002 |
| | Dual Expressing CHO, CAP IC₅₀ | | 0.27 | 0.068 |
| | (nM) | | | |
| Off | Kv7.3/5 EC₅₀ HCS (nM) | 3000 | >600 | 40900 |
| target | Kv7.4 EC₅₀ HCS (nM) | >2000 | 88000 | 49300 |
| | hERG IC₅₀ HCS (nM) | >2500 | >2650 | 18700 |
| | HepG2 Toxicity LD₅₀ (nM) | >1000 | 3370 | >16600 |
| in | LogD | 5.45 | 4.85 | 3.55 |
| vitro | Kinetic Sol. [mM] | 3.4 | 6 | 37 |
| ADME | PPB fu (human) | | | 0.3% |
| | PPB fu (rat) | 0.04% | 0.04% | 0.29% |
| | Microsome (human) | 55.5 | 8.6 | 64.78 |
| | CLint [mL/min/kg] (T_{1/2} min) | (25) | (145) | (19.25) |
| | Microsome (rat) | 177.2 | 63.5 | 527.2 |
| | CLint [mL/min/kg] (T_{1/2} min) | (14.1) | (39.3) | (4.7) |
| | Hepatocytes (human) | | 189.9 | 73.8 |
| | CLint [mL/min/kg] (T_{1/2} min) | | (20.3) | (52.2) |
| | Hepatocytes (rat) | | 513.8 | 1138.9 |
| | CLint [mL/min/kg] (T_{1/2} min) | | (12.6) | (5.7) |
| in | PK: Cl (mL/min/kg) | 38.5 | 65.8 | 35.7 |
| vivo | PK: Oral BA | 10.6 | 25.3 | 28.8 |
| ADME | Brain: Plasma 4-6 hours post-dose | | 199% | 4% |
| | DRG: plasma 4-6 hours post-dose | | 293% | 56% |

### Therapeutic Potential:

To better understand the landscape of potential clinical pain indications, compound 661 was used at 100 mg/kg and the reversal of pain was tested in several models at 6-hours post-dose. Models were selected where TRPV1 levels are increased in the DRG following injury/insult and where clear evidence exists of small to medium-sized DRG neurons being predominantly involved in pain neurotransmission; the Brennan post-operative pain (POP), distal tibial injury (DTI), monoiodoacetate OA (MIA), and chronic post-ischemic pain (CPIP) models were tested. The focus on small to medium-sized DRG neurons is because dual expression of Kv7.2 and TRPV1 is described to be localized in these neurons.

FIGS. 16K-O present bar graphs showing the efficacy profile in Paw withdrawal thresholds (PWT) assessed by Von-Frey (VF) measurements in the Spared Nerve Injury (SNI) (FIG. 16K), Distal Tibial Injury (DTI) (FIG. 16L), Chronic Post-Ischemia Pain (CPIP) (FIG. 16M), and Post-operative Pain Mechanical (POP-M) (FIG. 16N) and Post-operative Pain Thermal (POP-T) (FIG. 16O) models, 6 hours post-dosing of compound 661 (100 mg/kg) (light turquoise), compared at pre-dosing (dark turquoise) and baseline (grey).

As can be seen in FIG. 16K, oral administration of compound 661 at 100 mg/kg showed a statistically significant reversal of mechanical PWT in the SNI model at 6 hour post-dose (47.2%, P<0.0001). This effect size was similar to that observed with a 20 mg/kg dose (53% reversal, p<0.01; FIG. 16C), suggesting that a 50% reversal of PWT may represent the near-maximal achievable effect with this dual MOA.

As can be seen in FIGS. 16K-0, oral administration of compound 661 at 100 mg/kg showed that mechanical PWT tested at 6 hours post-dose was reversed in the SNI (47.2%, P<0.0001), DTI (45.9%, P<0.0001), and CPIP (27.6%, P<0.001) models compared to pre-dose PWTs on day 14 following model induction (FIGS. 16K-M).

As can be seen in FIGS. 16N-O, in the POP model one day post-surgery, compound 661 at 100 mg/kg did not affect the mechanical PWT, while the thermal withdrawal latency was completely reversed (P<0.0001), results supported by effects in this model using TRPV1 knockout mice (Barabas M E et al., Mol Pain, 2013; 9, 1744-8069-9-9, and Caterina et al., Science 2000; 288:306-313).

The monosodium iodoacetate-induced (MIA) model of osteoarthritis (OA) pain was performed 14 days following intra-articular MIA (3 mg) injection. Animals were dosed with naproxen (30 mg/kg) or compound 661 (100 mg/kg), and PWT to assess referred pain was measured at 6 hour post-dose in the ipsilateral hind paw. The obtained data is presented in FIGS. 16P-Q.

As can be seen in FIG. 16P, compound 661 showed a reversal of the PWT from 7.3 grams 1.1 pre-dose to 13.5 grams±2.4 post-dose (33% reversal, P<0.001), a similar effect size as the positive control drug naproxen.

As can be seen in FIG. 16Q, dynamic weight bearing (DWB) analysis, as a correlative measure of knee pain, showed that both naproxen (30 mg/kg) and compound 661 (100 mg/kg) exhibited comparable reversal of weight bearing changes compared to vehicle-treated rats at 6 hours post-dose (compound 661 44%, P<0.05).

### Example 6

### Metabolomics Studies on Compound 661

Metabolomics studies are a vital component of the drug development process, providing important insights into the metabolic profile of drug candidates. These studies are designed to identify and evaluate small molecules (metabolites) produced in-vivo. By conducting metabolomics studies, it is possible to evaluate the safety, metabolism, potential formation of reactive metabolites, and the potential for drug-drug interactions of a drug candidate.

### CYP Time-Dependent Inactivation (TDI) of Compound 661:

The time-dependent inactivation potency of compound 661 was evaluated.

The (−)NADPH/(+)NADPH ratio, a key measure for evaluating the potential for reactive metabolite formation and enzyme inhibition, was determined for Cytochrome P450 (CYP) enzymes CYP1A2, CYP2B6, CYP2C8, CYP2C9, CYP2C19, CYP2D36, and CYP3A4, and the results are presented in Table 5 below.

Seven CYP enzymes and eight substrates were tested to evaluate the TDI potential of compound 661. The TDI was identified by calculation of the (−)NADPH(+)NADPH ratio C₅₀ that is equal to or greater than 1.5. For the CYP3A4 enzyme in the presence of the substrate midazolam, the (−)NADPH((+)NADPH ratio was found to be 1.57, indicating a TDI of the CYP3A4 enzyme by compound 661 in the presence of midazolam.

TABLE 5

| | Compound 661 | | (−)NADPH/(+)NADPH |
|---|---|---|---|
| CYP/Substrate | IC₅₀ (μM) | | Ratio |
| CYP1A2_Phenacetin | (−)NADPH | >50 | |
| | (+)NADPH | >50 | |
| CYP2B6_Bupropion | (−)NADPH | 11.0 | 1.23 |
| | (+)NADPH | 8.91 | |
| CYP2C8_Amodiaquine | (−)NADPH | 14.3 | 0.866 |
| | (+)NADPH | 16.5 | |
| CYP2C9_Diclofenac | (−)NADPH | 14.8 | 1.14 |
| | (+)NADPH | 13.0 | |
| CYP2C19_S-Mephenytoin | (−)NADPH | 15.6 | 0.842 |
| | (+)NADPH | 18.5 | |
| CYP2D6_Dextromethorphan | (−)NADPH | 28.5 | 0.901 |
| | (+)NADPH | 31.7 | |
| CYP3A4_Midazolam | (−)NADPH | 30.5 | 1.57 |
| | (+)NADPH | 19.5 | |
| CYP3A4_Testosterone | (−)NADPH | 10.1 | 1.38 |
| | (+)NADPH | 7.36 | |

*IC₅₀ shift ratio (−NADPH/+NADPH pre-incubation) ≥1.5 is defined to be a TDI.

### Metabolite Identification (MET-ID) Assay on Compound 661:

A MET-ID assay was conducted for compound 661 using both rat and human hepatocytes, to characterize its metabolite profile in order to evaluate the potential for reactive metabolite formation.

The details of the metabolites identified are shown below in Table 6.

Thirteen different metabolites (M1-M13) were identified in the assay (shown in Table 6). Of note, metabolites M1-M7, representing 2.09% of the identified metabolites, exhibited glutathione conjugation that was attributed to indole oxidation. This conjugation could suggest the formation of reactive metabolites in hepatocytes exposed to compound 661.

TABLE 6

| | | | Relative Abundance | | | | |
|---|---|---|---|---|---|---|---|
| | | | (UV peak area | | UV peak area | | |
| | [M + H]⁺ | RT | % Total) | | (240-330 nM) | | |
| Code | m/z | (min) | Rat | Human | Rat | Human | Metabolic Pathways |
| M1 | 743.1293 | 5.81 | 0.21 | ND | #53 | #ND | Dechlorination, glutathione |
| | | | | | | | conjugation, di-oxygenation |
| | | | | | | | (P − Cl + GSH + 2O) |
| M2 | 727.1343 | 6.03 | 0.45 | + | 111 | + | Dechlorination, glutathione |
| | | | | | | | conjugation, mono-oxygenation |
| | | | | | | | (P − Cl + GSH + O) |
| M3 | 743.1295 | 6.71 | 1.23 | ND | 308 | #ND | Dechlorination, glutathione |
| | | | | | | | conjugation, di-oxygenation |
| | | | | | | | (P − Cl + GSH + 2O) |
| M4 | 727.1346 | 6.90 | 0.81 | 0.34 | 204 | 99 | Dechlorination, glutathione |
| | | | | | | | conjugation, mono-oxygenation |
| | | | | | | | (P − Cl + GSH + O) |
| M5 | 745.1005 | 8.82 | 2.37 | 0.08 | 594 | 25 | Glutathione conjugation |
| | | | | | | | (P + GSH) |
| M6 | 745.1010 | 8.99 | 0.69 | 0.63 | 172 | 186 | Glutathione conjugation |
| | | | | | | | (P + GSH) |
| M7 | 745.1008 | 9.12 | 1.72 | 1.04 | 429 | 306 | Glutathione conjugation |
| | | | | | | | (P + GSH) |
| M8 | 458.0431 | 9.94 | 5.79 | 31.45 | 1447 | 9229 | Hydrogenation, mono- |
| | | | | | | | oxygenation (P + 2H + O) |
| M9 | 472.0223 | 10.10 | 32.86 | 11.81 | 8213 | 3466 | Hydrogenation, oxidation |
| | | | | | | | (P + 2H + 2O − 2H) |
| M10 | 456.0277 | 10.38 | 1.07 | 1.16 | 267 | 339 | Mono-oxygenation (P + O) |
| M11 | 456.0276 | 10.48 | 22.01 | 10.20 | 5500 | 2992 | Mono-oxygenation (P + O) |
| M12 | 456.0278 | 10.62 | 1.97 | 1.13 | 493 | 331 | Mono-oxygenation (P + O) |
| M13 | 456.0275 | 10.97 | 2.07 | + | 516 | + | Mono-oxygenation (P + O) |

### Example 7

### Structure Activity Relationship (SAR) Optimization Studies Based on Compound 661

The CYP TDI and Met-ID assays of compound 661 (Example 6) suggested CYP304 inhibition and glutathione conjugation, attributed to indole oxidation. These findings suggest that the metabolites might be reactive and could potentially contribute to hepatotoxicity.

With the aim at minimizing off-target safety risk, reducing metabolic clearance and improving the solubility of compound 661, possible modifications were marked in three pharmacophoric sites: Ring A, Ring B, and the side chain, as shown in Scheme 2 below.

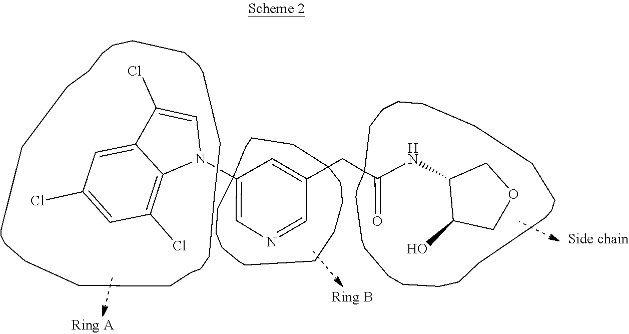

A library of preliminary compounds was synthesized and biological measurements and calculations were performed as described hereinabove.

The library included modifications that were aimed at reducing electron density in the indole moiety and reduce Log D, and include the following: Ring A modifications: replacing Ring A by a substituted indoline; and replacing the Ring A-7-chloro moiety by other groups such as trifluoromethyl and cyclopropyl, at the C-1 or C-2 positions.

Ring B modifications: replacing the pyridine by other nitrogen-containing heteroaryls such as pyridazine, pyrazine, pyrimidine, and triazine.

Side chain modifications: replacing the tetrahydroxytetrahydrofuran by acyclic groups such as hydroxyethyl.

The structures of exemplary newly designed compounds are presented in FIG. 17, and exemplary synthetic protocols are described hereinbelow and are shown in FIGS. 19-27. Overall, the compounds library included any coupling product of the acid precursors shown in FIG. 19 and the amine precursors shown in FIG. 20.

The data obtained for the compounds in this preliminary library (shown in Table 6) indicated that:
(i) Replacing the pyridine of Ring B by groups such as pyridazine and pyrimidine, resulted in reduced CYP inhibition values and reduced HLM (Human Liver Microsome) clearance.
(ii) Replacing the pyridine of Ring B by pyrimidine and triazine resulted in reduced Caco-2 Efflux ratio.
(iii) Replacing the tetrahydroxytetrahydrofuran of the side chain by hydroxyethyl reduced CYP inhibition values and reduced Caco-2 Efflux ratio.
(iv) Replacing the indole of Ring A by a substituted indoline results in increased HLM clearance.

The biological and physicochemical properties of 16 compounds selected from the library are presented in Table 7 below. These compounds were evaluated using various parameters including CYP-TDI, Met-ID human hepatocytes assay, Log D, HLM clearance, Caco-2 Efflux ratio, oral bioavailability, and dual hTRPV1 potency.

First, the toxicity of the compounds was assessed using the CYP-TDI and Met-ID human hepatocytes assays. Of the 16 tested compounds, nine (compounds 710, 740, 760, 767, 820, 830, 840, 843 and 851) demonstrated an acceptable toxicity profile, marking an improvement from compound 661. This was determined by a CYP-TDI of <1.5 and a MET-ID assay which did not indicate the presence of glutathione conjugation, a signal of potential reactive metabolites.

The metabolism and clearance of these compounds was evaluated using the HLM (T_{1/2}) and Rat pharmacokinetics (PK) clearance tests. Eight of the non-toxic compounds (compounds 740, 760, 767, 820, 830, 840, 843 and 851) showed improved HLM and/or Rat PK clearance rates compared to compound 661 (as shown in Table 6).

The absorbance of the compounds was then evaluated by measuring the Caco-2 Efflux ratio, with compound 661 displaying a ratio of 12.3, indicating suboptimal absorption. Five of the non-toxic and metabolically favorable compounds (760, 767, 820, 843, and 851) exhibited a Caco-2 Efflux ratio below the desired threshold of 3, indicating optimal absorption.

The dual hTRPV1 (nM) inhibition was evaluated for the remaining compounds, of which compounds 760, 767 and 843 exhibited the most pronounced inhibition at 0.201, 0.250 and 0.528 nM respectively.

TABLE 7

| | | | ADME/PK | | | | | Potency |
|---|---|---|---|---|---|---|---|---|
| | CYP-TDI | Human | | HLM | Caco-2 | | | Dual |
| | (midazolam) | Hepatocytes | | (T½ | Efflux | Rat PK Cl | Oral | hTRPV1 |
| | (<1.5) | MetID: | LogD | [min]) | ratio (<3) | (mL/min/kg) | BA | [nM] |
| NH91 | | | 4.66 | | | | | |
| 661 | 1.57 | + | 3.55 | 19.25 | 12.30 | 35.70 | 28.80 | 0.070 |
| 710 | 1.22 | | 2.88 | 10.1 | | | | |
| 720 | >1.6 | | 3.24 | 6.1 | | | | |
| 730 | >1.57 | | 3.22 | 9.9 | | | | |
| 740 | 1.14 | | 3.28 | 48.5 | 80.20 | | | |
| 750 | 1.39 | + | 3.29 | 19.10 | | | | 0.100 |
| 760 | 0.00 | None | 3.28 | 23.5 | 2.10 | 60.10 | 48.60 | 0.201 |
| 767 | 1.20 | None | 3.30 | 61.9 | 0.90 | 42.50 | 79.70 | 4 |
| 770 | >1.9 | | 3.88 | 34.8 | | | | |
| 790 | 2.04 | | 4.29 | 26.7 | | | | |
| 800 | >2.67 | | >4.1 | 15.4 | | | | |
| 810 | >5.21 | | 4 | 50.7 | | | | |
| 820 | <0.9 | None | 3.82 | 33.8 | 2.49 | | | 48.0 |
| 830 | 0.00 | | 3.2 | 58.1 | 84.02 | | | |
| 840 | 0.00 | None | 3.63 | 52.0 | 5.4 | | | 479.1 |
| 843 | 0.00 | 0.2% | 3.75 | >145 | 0.8 | 6.6 | 73.4 | 0.528 |
| 851 | 0.00 | | 3.38 | 60.6 | 0.9 | | | 1075.0 |

Met-ID is indicative of nucleophile trapping tox risk
BA = Bioavailability

Compound 760 has a molecular weight of about 440 grams/mol, c Log P of 3.4, Log D of 3.28, kinetic solubility of 57 μM, and the Ligand lipophilicity efficiency—LLE—of 6.4.

Compound 760 displays a HLM T½ of 48.1 minutes and a RLM T½ of 11.9 minutes; Caco-2 AB of 7.6; and efflux ratio of 2.1.

Compound 760 was selected from further studies.

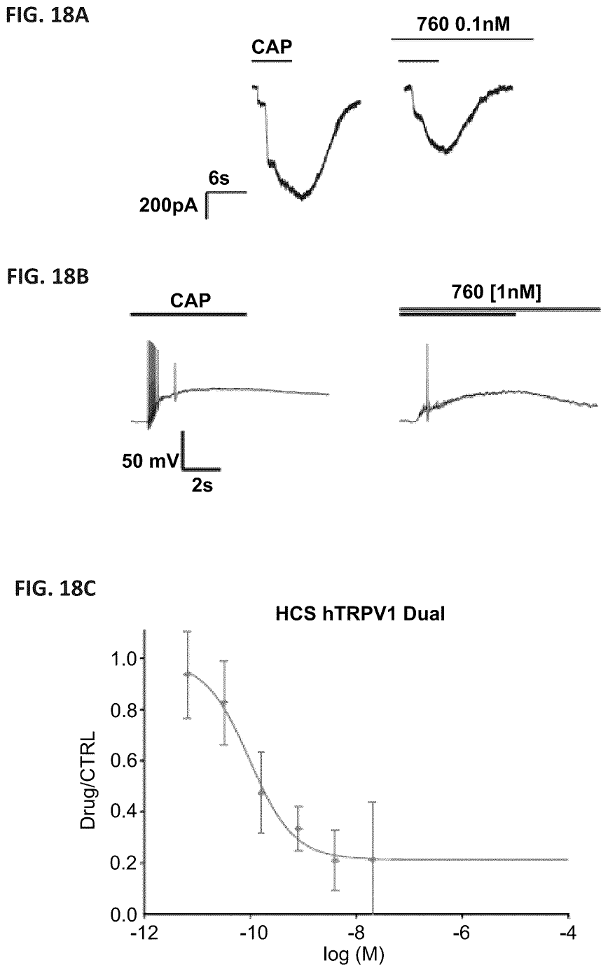

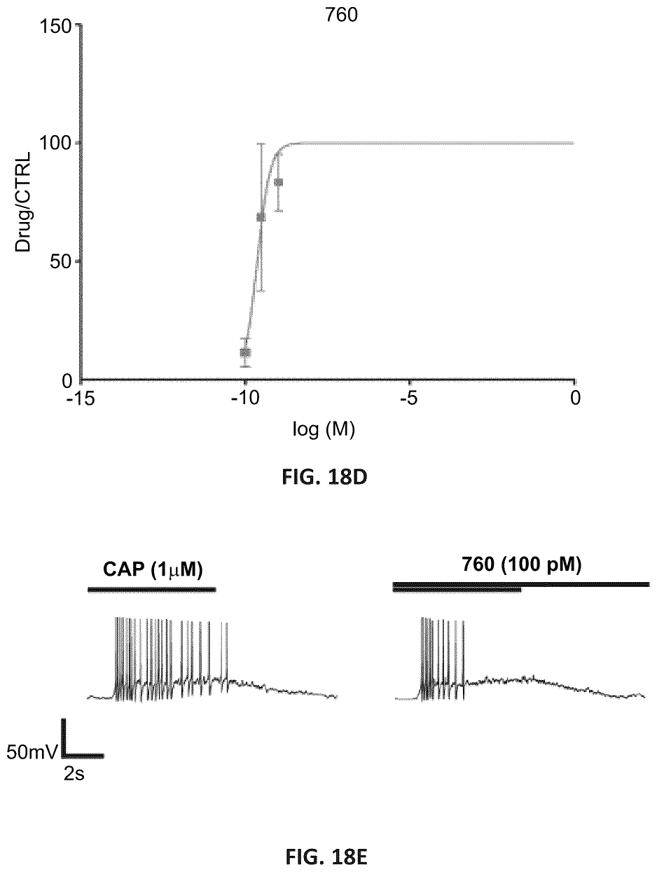

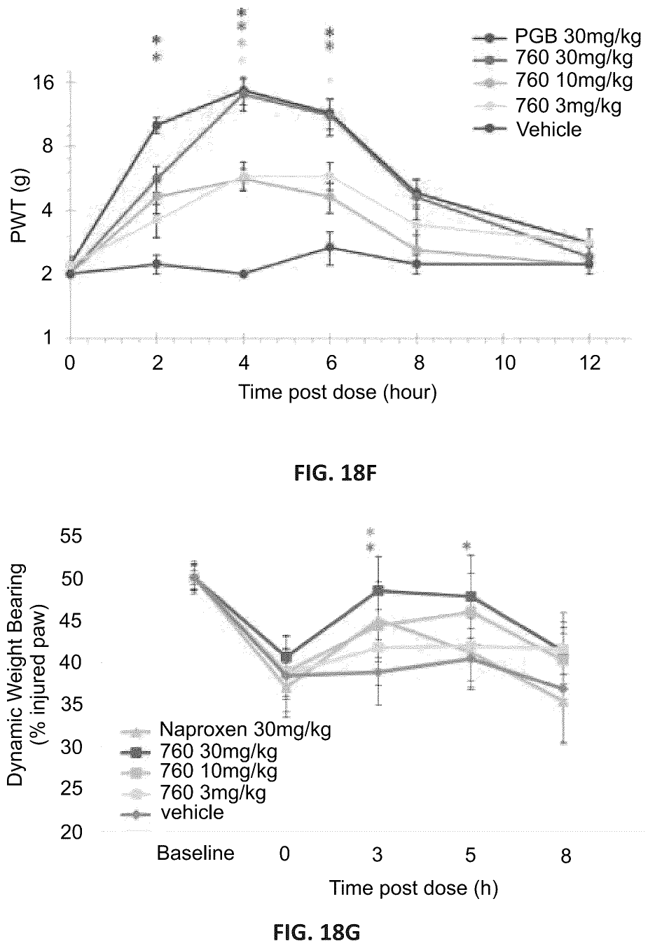

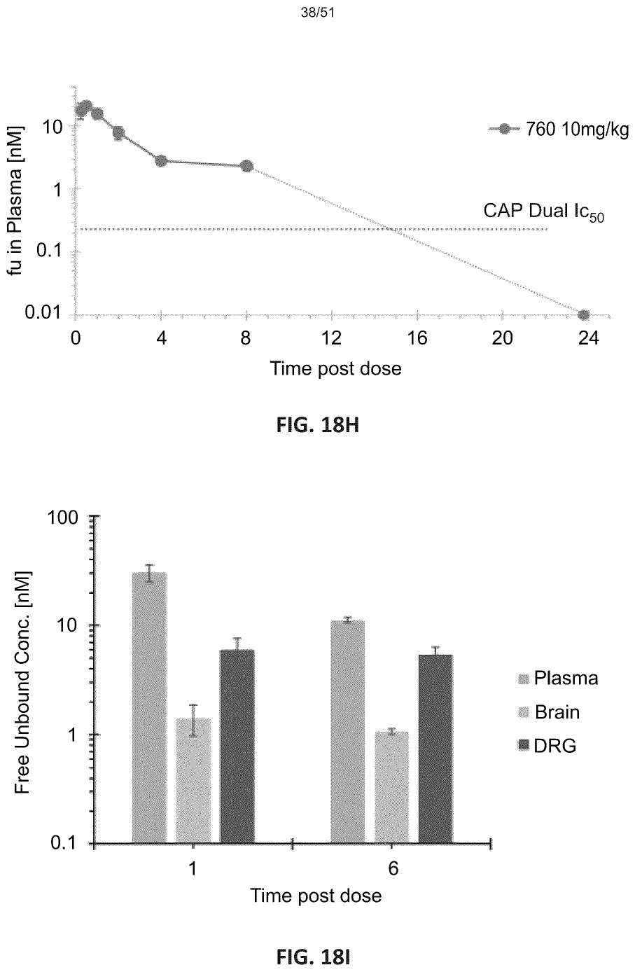

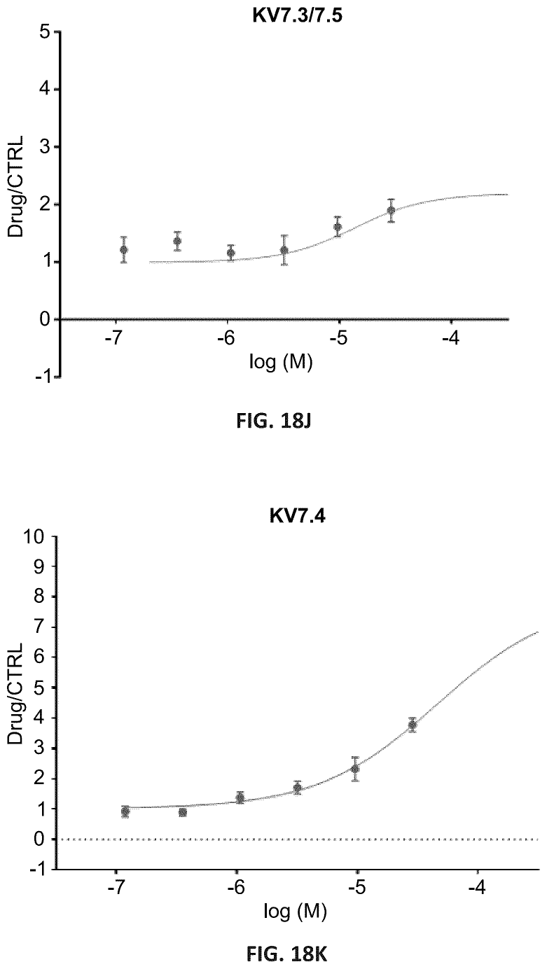

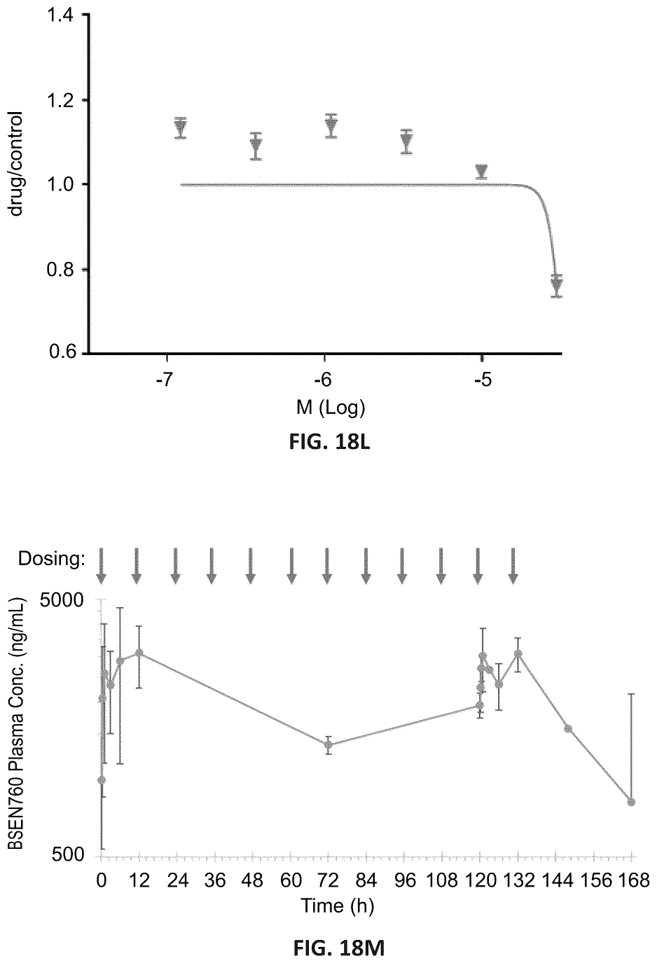

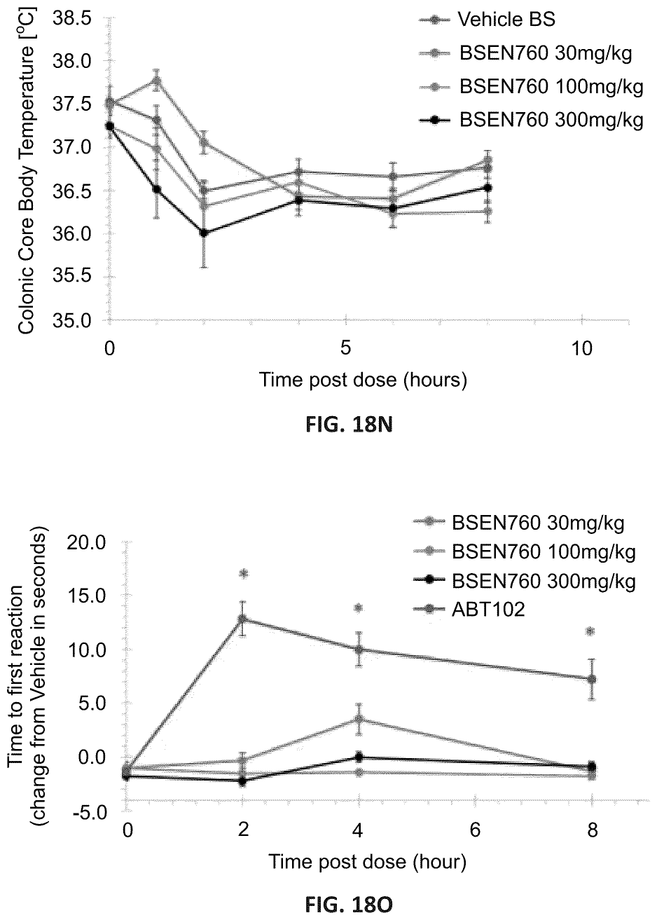

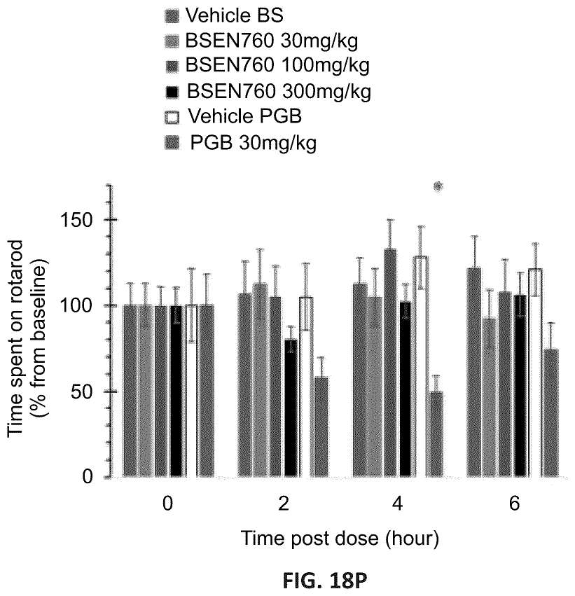

FIG. 18A presents whole-cell currents electrophysiology measurements in CHO cells co-expressing hKv7.2/3 and hTRPV1 following hKv7.2/3 activation, in the presence of compound 760 at 0.1 nM.

FIG. 18B presents data obtained for rat DRG neuron firing in response to capsaicin application with exemplary recording using compound 760 at 1.0 nM, showing about 90% inhibition of action potential firing.

FIG. 18C presents a plot showing the quantification of capsaicin-induced TRPV1 activity inhibition in CHO cells co-expressing Kv7.2/3 and TRPV1 CHO cells, by compound 760.

FIG. 18D presents a plot showing the quantification of rat DRG neuron's capsaicin-induced action potential inhibition by compound 760, presented as the drug over control average % inhibition dose-response.

FIG. 18E presents data obtained for rat DRG neuron firing (I=0) with Capsaicin application (CAP, 1 μM, 6 seconds) stimulation before or after compound 760 application (0.1 nM).

Compound 760 has an IC₅₀ value of 0.23 nM when examined on rat DRG sensory neurons, inhibiting the electrical response to capsaicin, the pain analogue stimulation. This was supported by the IC₅₀ of hTRPV1 current inhibition in CHOs with both targets co-expressed and activated, as measured in electrophysiology assays (EP), which was about 100 μM (46.0±13.7% inhibition at 100 μM; N=4), suggesting that dual activity of this compound is exhibited at IC₅₀ of 100-230 μM.

### In-Vivo Analysis of Compound 760:

FIG. 18F presents an efficacy profile of compound 760 in the Spared Nerve Injury (SNI) model at doses of 3 mg/kg, 10 mg/kg and 30 mg/kg compared to a vehicle and pregabalin (30 mg/kg) controls.

FIG. 18G presents an efficacy profile of compound 760 in the osteoarthritic pain MIA model at doses of 3 mg/kg, 10 mg/kg and 30 mg/kg, compared to a naproxen (30 mg/kg, PO) control.

As can be seen, compound 760 provided pain relief comparable to Pregabalin's high and impairing dose in the rat SNI neuropathic pain model (760 effective at 3-30 mg/kg PO).

Compound 760 provided pain relief also in the rat osteoarthritic pain model, superior in both effect and duration to high dose of naproxen (760 effective at 3-30 mg/kg PO).

FIG. 18H presents a scatter plot showing the rat PK exposure of compound 760 following oral delivery of 10 mg/kg, at 1 and 6 hours post dose.

FIG. 18I presents a bar graph showing the rat PK exposure of compound 760 (green), brain (yellow) and DRG (blue) following oral delivery of 10 mg/kg, at 1 and 6 hours post dose.

As can be seen, compound 760 displayed PK clearance of 60.1 mL/min/kg with T½ of 2.68 hours, and Volume of distribution at steady state (Vdₛₛ) of 2.37 L/Kg.

Compound 760's oral bioavailability was 48.6%.

Compound 760 displayed low CNS exposure, with a fraction unbound brain-to-plasma ratio of 9.6%, while for the target organ the unbound DRG-to-plasma ratio of 48.5% (1-6 hours post-dose).

### Toxicology:

To address the specificity of compound 760, a wide functional screening panel for 760 against 98 targets was performed, showing a clean off-target effect profile, as presented in Table 8. Additionally, up to concentration of 10 micromolar of compound 760, no effect was observed on the following targets: hERG, Kv7.1, Nav1.5, NMDA, nAChR(a4/b2), GABAA, Dopamine D1-D5, Vasopressin 1-2, Bradykynin B1-2, Cannabinoid CB1-2, Opioid-delta, kappa, mu, COX-1/2.

TABLE 8

| | IC/EC₅₀ |
|---|---|
| Target | (nM) |
| Sigma1 | >50000 |
| Sigma2 | 842.5 |
| PDE4D2 | 13757 |
| 5-HTT | 29459 |
| DAT | >50000 |
| Cav1.2 | 2990 |
| Adrenergic β2-antagonist | 45953 |
| 5-HT1A-antagonist | 30362 |

FIG. 18J presents a plot showing the activation of hKv7.3/5 activity, measured using fluorescent assay, by compound 760, presented as drug over control-dose response. EC50 was 10.7 μM, as measured by Thallium ions (Tl+) fluorescence.

FIG. 18K presents a plot showing the activation of hKv7.4 activity, measured using fluorescent assay, by compound 760, presented as drug over control-dose response. EC50 was 46.2 μM, as measured by Thallium ions (Tl+) fluorescence.

FIG. 18L presents a plot showing activation of hERG activity, measured using fluorescent assay, by compound 760, presented as drug over control-dose response. EC50 was 33.4 μM, as measured by Thallium ions (Tl+) fluorescence.

As can be seen, compound 760 lacks any effect on hERG and Kv7.3/4/5 activity below 10 μM.

### CYP and Time-Dependent Inhibition (TDI):

To evaluate any potential risk of drug-drug interaction (DDI), compound 760 was tested for its CYP inhibition and TDI ratio. The results are presented in Table 9 below and show that compound 760 did not show significant CYP inhibition or time-dependent inhibition (TDI) (ratio <1.5) up to 20 micromolar.

Additionally, compound 760 was tested for its capability to act as a transporter substrate for various transporters, and was not found to be a transporter substrate for any of the tested transporters. More specifically, compound 760 was not a transporter substrate or a poor substrate of P-gp, BCRP, and was not a transporter substrate of OATP1B1, OATP1B3, OATP2B1, OAT1, OAT3, OCT1, OCT2, MATE1 and MATE2-K.

TABLE 9

| | CYP | |
|---|---|---|
| | inhibition | |
| | IC₅₀ | CYP TDI ratio |
| | (μM) | (−)NADPH/(+)NADPH |
| CYP1A2 | >50 | — |
| CYP2B6 | 34.9 | 1.42 |
| CYP2C8 | 49.2 | 1.34 |
| CYP2C9 | 44.3 | 1.07 |
| CYP2C19 | >50 | — |
| CYP2D6 | >50 | — |
| CYP3A- | >50 | — |
| Midazolam | | |
| CYP3A- | 19.8 | 1.15 |
| Testosterone | | |

### In Vitro Micronucleus:

The micronucleus assay was performed according to the regulatory guidelines. In brief, CHO-K1 cells were plated at 10000 cells/well in 3 plates and incubated In CHO-K1 growth medium (containing F12K, 10% FBS and 1% PS) overnight. On the following day, compound 760 (0.03-200 μM in 0.5% DMSO) or DMSO 0.5% only were added, and incubation continued for 20-24 hours.

As a control for the S9 treatment, plate No. 1, cells were treated with cyclophosphamide as a positive control, for 3 hours. For plate No. 2, without S9 treatment, cells were treated with mitomycin C as a positive control, compounds for 3 hours, and for plate No. 3, without S9 treatment, cells were treated with bleomycin sulfate as a positive control continuously for 24 hours.

On the following day, 6 μg/mL of cytochalasin B were added to each well and plates were further incubated overnight. In the day after medium was replaced with a combined 10 μg/mL Hoechst dye solution and 0.5 μM calcein-AM in 100 μL warm DPBS for 30 minutes and the plates were scanned using CQ1 high-content analysis system.

The obtained data is presented in Table 10 below and show that compound 760 lacks significant micronucleus formation and thus shows low potential for mutagenicity.

TABLE 10

| | | Cytotoxicity | Micronucleus |
|---|---|---|---|
| | | (%) | response |
| | | Absolute | Absolute |
| | | EC₅₀ | MEC |
| No. | Cpd ID | (μM) | (μM) |
| Plate 1 with S9 | Cyclophosphamide | 41.39 | >400 |
| | Compound 760 | >200 | >200 |
| Plate 2 | Mitomycin C | >16 | >16 |
| | Compound 760 | >200 | >200 |
| Plate 3 | Bleomycin sulfate | 1.59 | >28 |
| | Compound 760 | 81.16 | >200 |

### Mini Ames Assay:

The Mini-Ames assay was performed according to the regulatory guidelines. In brief, 5 strains were selected for testing: Salmonella typhimurium (TA98, TA100, TA1535, TA1537) and Escherichia coli WP2uvrA (pKM101). A standard six-well culture plate with top agar containing 0.6% (w/v) agar and 0.5% (w/v) sodium chloride was supplemented with 0.5 mM D-biotin and 0.5 mM L-histidine for Salmonella typhimurium strains or 0.5 mM D-biotin and 0.5 mM L-tryptophan for Escherichia coli WP2uvrA (pKM101). Compound 760, at 6 concentrations within a range of 15-500 g/well was added in triplicates for each strain, with and without S9 conditions. Average number of revertants for each dose group condition was compared to negative control, and cytotoxicity and precipitates were recorded.

The obtained data, showed that compound 760 did not induce substantial dose-dependent increases in reversion rates on all the 5 strains tested both with and without S9. No significant cytotoxic effect was observed in all five strains, both in the absence and presence of S9. No obvious precipitates were observed at the tested concentration range with or without S9 in any tested strains. These data further support that compound 760 has low potential for mutagenicity.

### PK Studies:

FIG. 18M presents a scatter plot showing rat PK exposure of compound 760 following oral delivery of up to BID 300 mg/kg, over 168 hours. Chronic 5-day BID exposure of up to 300 mg/kg did not raise any gross safety concerns in rats, during the in-life of the study or following terminal gross necropsy.

FIG. 18N presents a scatter plot for the colonic core body temperature (° C.) in rats treated with compound 760 at doses of 30 mg/kg (green), 100 mg/kg (grey) and 300 mg/kg (black), compared to a vehicle control, over a time period of 8 hours.

FIG. 18O presents a scatter plot for the Time to first reaction (change from vehicle in seconds) in rats treated with compound 760 at doses of 30 mg/kg, 100 mg/kg and 300 mg/kg, compared to ABT-102 (10 mg/kg) positive control.

FIG. 18P presents a bar graph for the Time spent on rotarod (% from baseline) in rats treated with compound 760 at doses of 30 mg/kg, 100 mg/kg and 300 mg/kg, compared to a vehicle, vehicle-pregabalin, and pregabalin (30 mg/kg) controls.

As can be seen, compound 760 displayed safety potential at up to 300 mg/kg oral dosing, when measuring rat core-body temperature (thermoregulation), hot-plate response (thermosensation) compared to ABT-102 (10 mg/kg) positive control and motor coordination and sedation, using rotarod and compared to pregabalin (30 mg/kg) positive control.

Table 11 below summarizes the data obtained in the course of the drug design and development, en route to identifying lead candidates.

TABLE 11

| | | | Compound | Compound | Compound | Compound |
|---|---|---|---|---|---|---|
| | | NH91 | 421 | 627 | 661 | 760 |
| Chemical | Aniline | Yes | No | No | No | No |
| properties | structural | | | | | |
| | alert | | | | | |
| | TDI related | NT | Yes | Yes | Yes | No |
| | reactive | | | | | |
| | metabolite | | | | | |
| | MWt | 427.4 | ~420 | ~415 | ~440 | ~440 |
| | clogP | 5.34 | 4.5 | 4 | 2.6 | 3.4 |
| | logD | 4.66 | 5.45 | 4.85 | 3.55 | 3.28 |
| | tPSA | 61 | 54 | 54 | 76 | 89 |
| | CNS MPO | 2.8 | ~3.2 | ~3.6 | ~4.6 | ~4.1 |
| Potency | Rat DRGs | 500 | 25 | 0.1 | 0.002 | 0.23 |
| | IC₅₀ (nM) | | | | | |
| | LLE | 1.6 | 2.1 | 5.2 | 6.4 | 6.4 |
| in vitro | Kinetic Sol. | 20 | 3.4 | 6 | 37 | 57 |
| ADME | [mM] | | | | | |
| | HLM T½ | 7.4 | 25 | 145 | 19.3 | 48.1 |
| | (min) | | | | | |
| | RLM T½ | 2.5 | 14.1 | 39.3 | 4.7 | 11.9 |
| | (min) | | | | | |
| | Caco-2 AB | NA | NA | NA | 1.78 (12.3) | 7.6 (2.1) |
| | (e.r.) | | | | | |
| in vivo | PK: Cl | NA | 38.5 | 65.8 | 35.7 | 60.1 |
| ADME | (mL/min/kg) | | | | | |
| | PK: Oral BA | NA | 10.6 | 25.3 | 28.8 | 48.6 |
| | PK: T_{1/2} | NA | NA | 1.48 | 2.49 | 2.68 |
| | (hours) | | | | | |
| | Brain: | NA | NA | 199% | 4% | 36% |
| | Plasma 4-6 hr | | | | | |
| | post-dose | | | | | |
| | DRG: | NA | NA | 293% | 56% | 180% |
| | plasma 4-6 hr | | | | | |
| | post-dose | | | | | |

### Example 8

### Chemical Synthesis

### Synthesis of Compounds 421-6 to 851

Compounds 421-6 to 851 were prepared generally through conjugation of acid precursors, as presented in FIG. 19, with amine precursors, as presented in FIG. 20, in a synthetic protocol comprising five or six chemical steps. FIG. 19 presents the structures of exemplary acid precursors used in the synthetic protocol. FIG. 20 presents the structures of exemplary amine precursors used in the synthetic protocol.

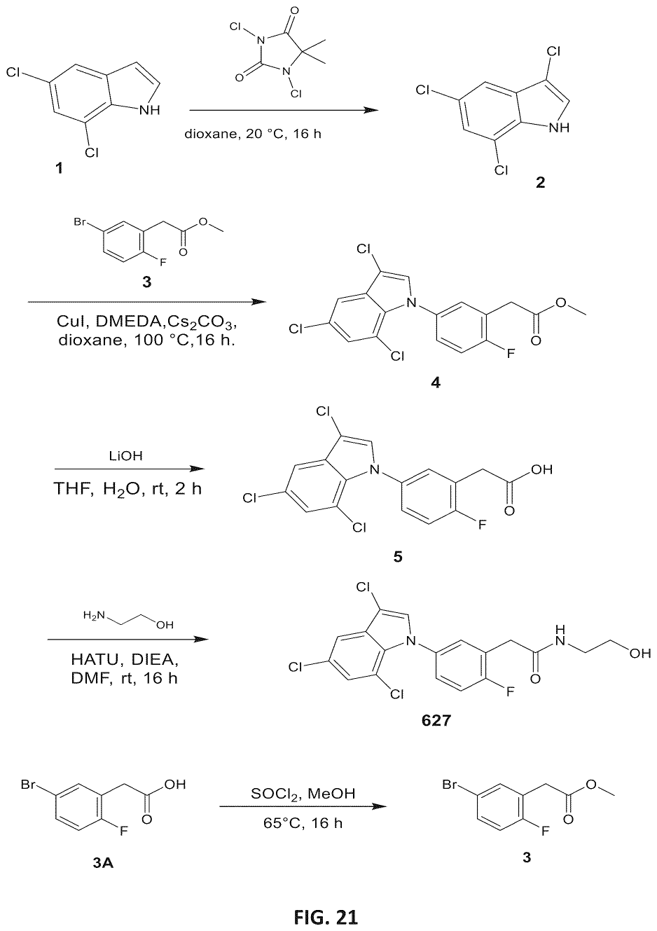

An exemplary synthetic protocol of Chemotype 3 compound 627 is presented in FIG. 21.

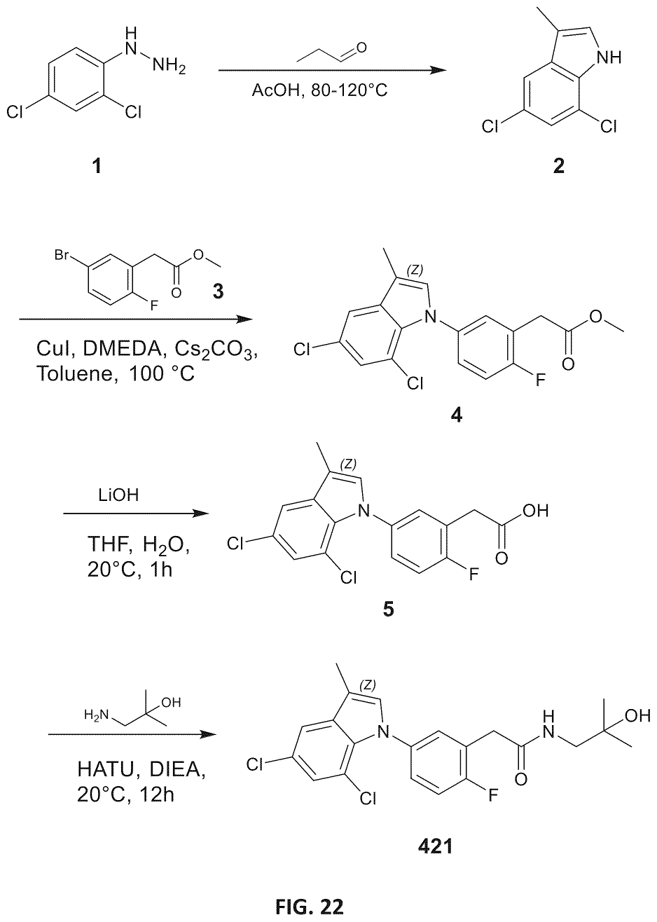

An exemplary synthetic protocol of Chemotype 3 compound 421 is presented in FIG. 22.

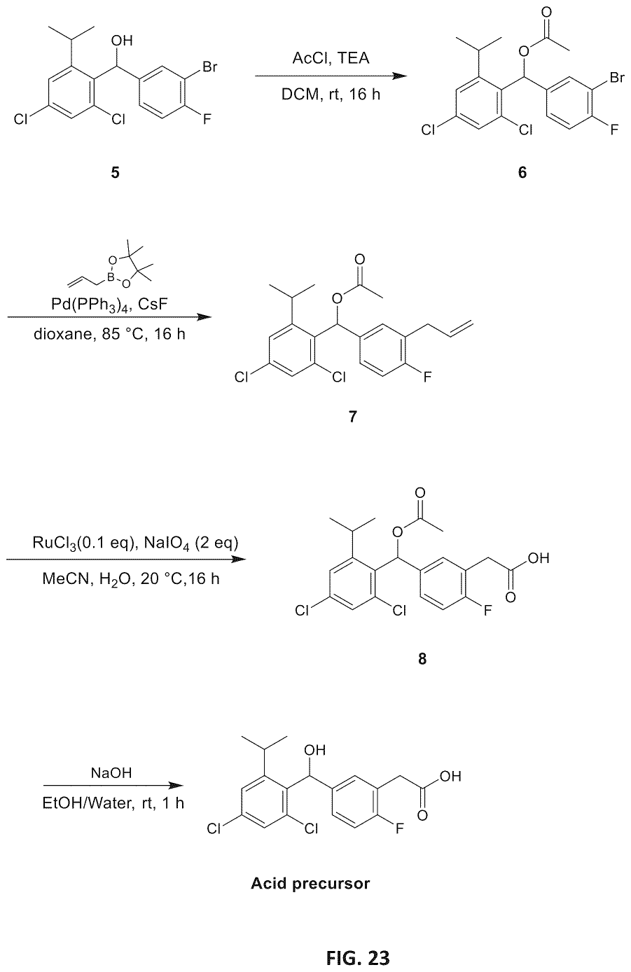

An exemplary synthetic protocol for an acid precursor used in the synthetic protocol of Chemotype 3 compounds is presented in FIG. 23.

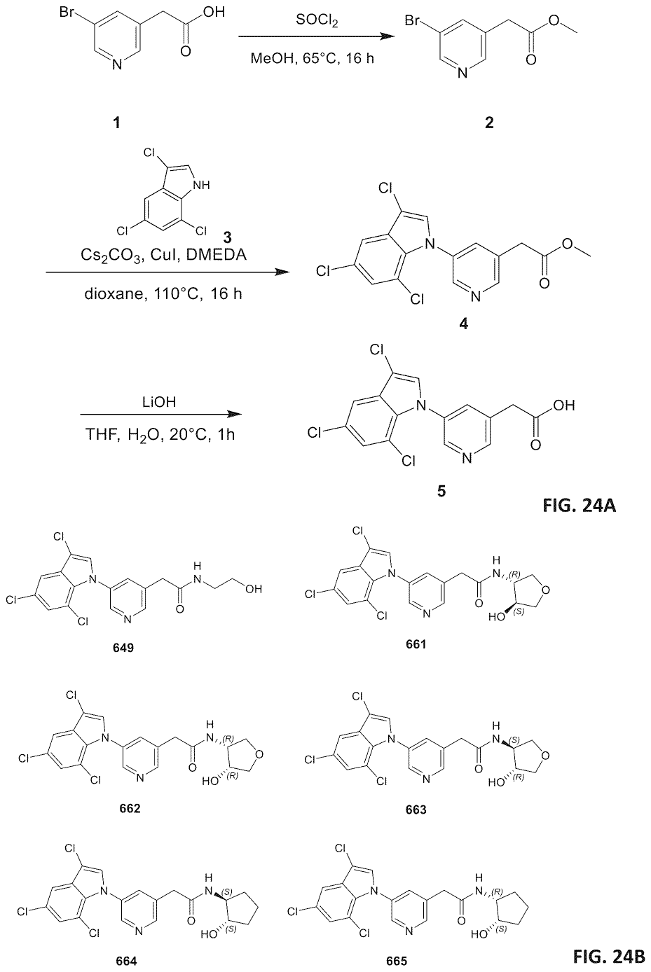

An exemplary synthetic protocol for an acid precursor used in the synthetic protocol of Chemotype 1 is presented in FIG. 24A. FIG. 24B presents structures obtained by coupling the acid precursor of FIG. 24A with amine precursors.

### Synthesis of Compounds 762, 763, 770, 770_3, 843 and 844:

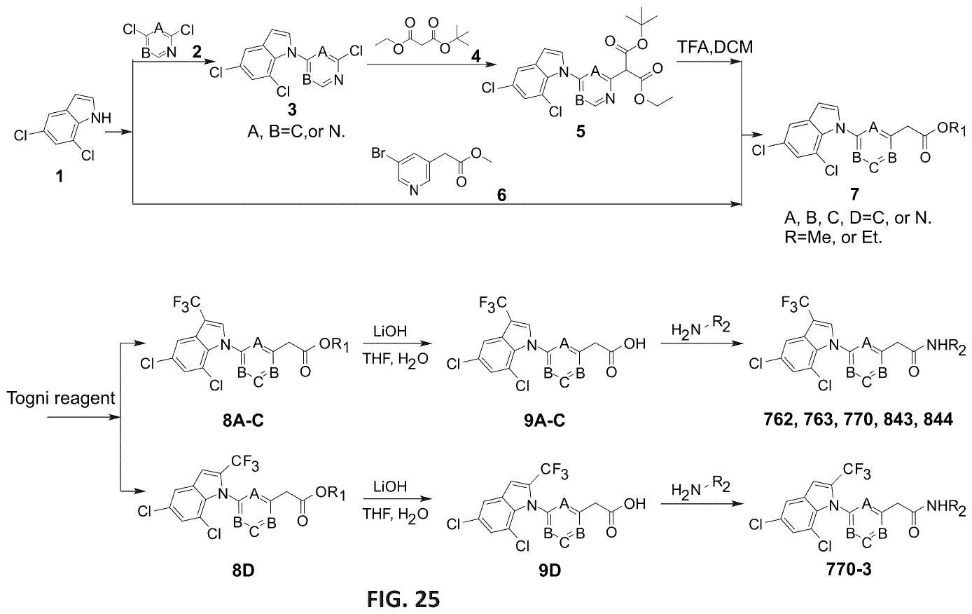

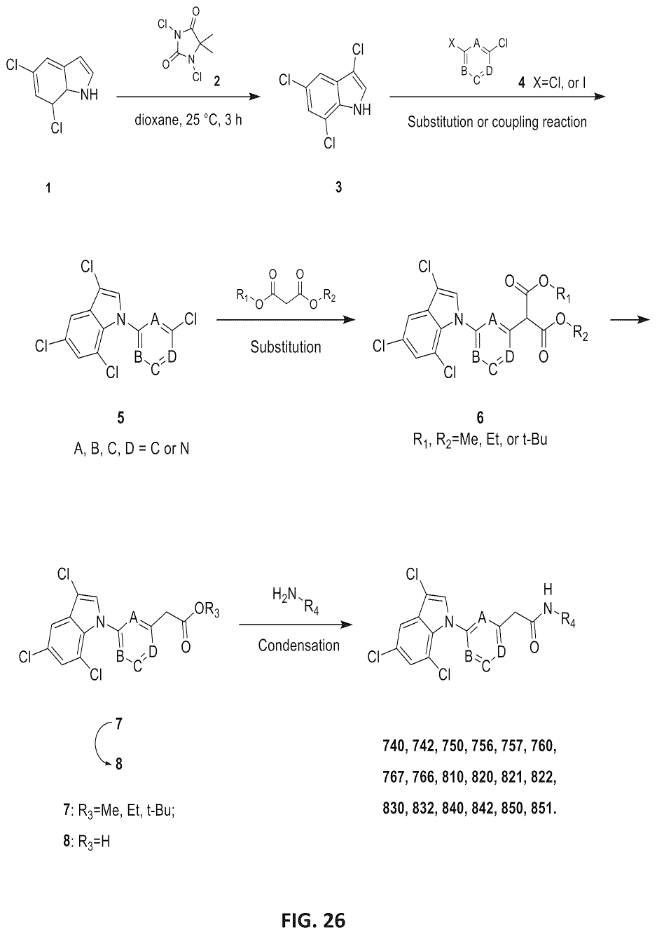

Compounds 762, 763, 770, 770_3, 843, and 844 were prepared generally through conjugation of acid precursors, as presented in FIG. 19, with amine precursors, as presented in FIG. 20, in a synthetic protocol as presented in FIG. 25. In the structures presented in FIGS. 25 and 26, variable A corresponds to variable A in Formula I, variable B corresponds to variables E and B in Formula I, and variable C corresponds variable D in Formula I.

The following are exemplary synthetic protocols.

### General Procedure for Preparation of Compound 3 of FIG. 25:

To a solution of compound 1 (1 mol equivalent) and compound 2 (1.1 mol equivalent) in a solvent was added base (2.5 mol equivalents). The reaction mixture was stirred at appropriate temperature for 1 to about 16 hours. TLC showed that starting material 1 was consumed. The reaction was poured into ice water, and the obtained mixture was extracted with ethyl acetate (EA) three times. The combined organic layer was washed with brine, dried over Na₂SO₄, filtered and concentrated under reduced pressure to dryness to give a crude product, which was purified by silica gel column chromatography to give compound 3.

Compound 3A was obtained using 2,4-dichloropyrimidine as material, and K₂CO₃ as base in DMF solution (85° C., 16 hours, 77% yield).

LCMS: Retention time (RT)=0.640 minutes, M/Z=299.6 [M+H]⁺

Compound 3B was obtained using 4,6-dichloropyrimidine as material, and Cs₂CO₃ as base in MeCN solution (80° C., 1 hour, 41% yield).

¹H-NMR (400 MHz, CDCl₃): δ (ppm)=8.94 (s, 1H), 7.69 (d, J=3.6 Hz, 1H), 7.59 (s, 1H), 7.34 (s, 1H), 7.27 (s, 1H), 6.73 (d, J=3.6 Hz, 1H).

LCMS: RT=0.640 minutes, M/Z=299.9 [M+H]⁺

### General Procedure for Preparation of Compound 5 of FIG. 25:

To a solution of compound 3 (1 mol equivalent) and compound 4 (2 mol equivalents) in DMF was added Cs₂CO₃ (2.5 mol equivalents). The reaction mixture was stirred at 80° C. for 4-16 hours, and was monitored by LCMS. Once completed, the reaction mixture was poured into ice water, and extracted with EA three times. The organic layer was washed with brine, dried over Na₂SO₄, filtered and concentrated under reduced pressure to dryness to give a crude product, which was purified by silica gel column chromatography to give compound 5.

Compound 5A was obtained using 3A as a starting material (80° C., 16 hours 79% yield). LCMS: RT=0.828 minutes, M/Z=450.1 [M+H]⁺

Compound 5B was obtained using 3B as a starting material (80° C., 4 hours, 68% yield).

¹H-NMR (400 MHz, CDCl₃): δ (ppm)=9.11 (s, 1H), 7.69 (d, J=3.2 Hz, 1H), 7.58 (d, J=2.0 Hz, 1H), 7.47 (d, J=0.9 Hz, 1H), 7.31 (d, J=2.0 Hz, 1H), 6.72 (d, J=3.2 Hz, 1H), 4.87 (s, 1H), 4.35-4.20 (m, 2H), 1.49 (s, 9H), 1.31 (t, J=7.2 Hz, 3H).

LCMS: RT=0.677 minutes, M/Z=450.1 [M+H]⁺

### General Procedure for Preparation of Compound 7 of FIG. 25:

Method A: To a solution of compound 5 in DCM was added TFA (V_{DCM}:V_{TFA}=1:1) at 0° C. The mixture was stirred at 25° C. for 2 hours. TLC indicated compound 5 was consumed and a spot was formed. The mixture was concentrated under reduced pressure to remove the solvent. The residue was diluted with EA and washed with NaHCO₃, dried over Na₂SO₄, filtered and concentrated under reduced pressure, and the obtained crude residue was purified by column chromatography to give compound 7.

Method B: To a solution of compound 1 (1.0 mol equivalent) and compound 6 (1.1 mol equivalent) in dioxane was added CuI (0.1 mol equivalents), DMEDA (0.2 mol equivalents) and K₂CO₃ (2.0 mol equivalent) and the mixture was degassed with N₂ three times, and thereafter was stirred at 100° C. for 16 hours. The reaction mixture was then diluted with water and extracted with EA. The combined organic layers were washed with brine, dried over Na₂SO₄, filtered and the filtrate was concentrated under reduced pressure. The residue was purified by column chromatography to give compound 7.

Compound 7A was obtained by method A using 5A as a starting material (95% yield).

LCMS: RT=0.625 minutes, M/Z=350.0 [M+H]⁺

Compound 7B was obtained by method A using 5B as a starting material (94% yield).

¹H NMR: (400 MHz, CDCl₃) δ (ppm)=9.13 (s, 1H), 7.71 (d, J=3.2 Hz, 1H), 7.59 (s, 1H), 7.32 (d, J=12.4 Hz, 1H), 7.27 (s, 1H), 6.73 (d, J=3.2 Hz, 1H), 4.23 (q, J=7.2, 12.4 Hz), 3.94 (s, 2H), 1.27 (t, J=7.2 Hz, 3H).

LCMS: RT=0.618 minutes, M/Z=350.1 [M+H]⁺

Compound 7C was obtained by method B (34% yield).

LCMS: RT=0.640 minutes, M/Z=335.2 [M+H]⁺

¹H-NMR (400 MHz, DMSO-d₆): δ (ppm)=8.63 (d, J=2.4 Hz, 1H), 8.59 (d, J=2.0 Hz, 1H), 7.86 (t, J=2.0 Hz, 1H), 7.77 (d, J=1.6 Hz, 1H), 7.66 (d, J=3.2 Hz, 1H), 7.32 (d, J=1.6 Hz, 1H), 6.80 (d, J=3.2 Hz, 1H), 3.87 (s, 2H), 3.65 (s, 3H).

### General Procedure for Preparation of Compound 8 of FIG. 25:

To a solution of compound 7 (1 mol equivalent) in DCM Togni reagent (1-(trifluoromethyl)-1,2-benziodoxol-3-one, 2 mol equivalents) and dichloroiron (0.2 mol equivalent) were added at 25° C. under N₂. The mixture was stirred at 35° C. for 24 hours. TLC indicated compound 7 was remained and four spots were formed (regio-isomers obtained from the substitution of Ar—H with CF3 group). The mixture was thereafter filtered and the filtrate was concentrated under reduced pressure to give a crude residue which was purified by column chromatography followed by preparative HPLC to give compound 8 and its isomers respectively, as confirmed by HMBC.

Compound 8A was obtained using 7A as a starting material (3.5% yield).

LCMS: RT=0.666 minutes, M/Z=417.8 [M+H]⁺

HPLC: RT=3.797 minutes, purity=95.0%

¹H-NMR (400 MHz DMSO-d6): δ (ppm)=9.06 (d, J=5.6 Hz, 1H), 8.58 (s, 1H), 7.86 (d, J=5.2 Hz, 1H), 7.56 (s, 1H), 7.66 (d, J=1.6 Hz, 1H), 4.11 (q, J=7.2 Hz, 14 Hz, 2H), 4.05 (s, 2H), 1.17 (t, J=7.2 Hz, 3H).

Special NMR (400 MHz DMSO-d6): H6 is related to C2 in HMBC, H8 is related to C12 in HMBC and C8:132.0 ppm confirmed the structure.

Compound 8B was obtained using 7B as a starting material (2.8% yield).

¹H-NMR (400 MHz, CDCl₃): δ (ppm)=9.15 (s, 1H), 7.97 (s, 1H), 7.72 (s, 1H), 7.43 (s, 1H), 7.40 (s, 1H), 4.30-4.20 (m, 2H), 3.94 (s, 2H), 1.35-1.20 (m, 3H).

2D NMR: 19F is correlated with H11 and H9 in NOE.

Compound 8C was obtained using 7C as a starting material (3.1% yield). 8D was obtained as regio-isomers.

¹H NMR (400 MHz, Methanol-d₄): δ (ppm)=9.05 (s, 1H), 8.93 (s, 1H), 8.61-8.49 (m, 1H), 8.15 (s, 1H), 7.72 (s, 1H), 7.44 (d, J=1.6 Hz, 1H), 4.08-4.00 (m, 2H), 3.76 (s, 3H).

Compound 8D was obtained using 7C as a starting material (3.1% yield).

¹H-NMR (400 MHz, METHANOL-d₄): δ (ppm)=8.80 (d, J=9.6 Hz, 2H), 8.21 (s, 1H), 7.78 (d, J=1.6 Hz, 1H), 7.36 (d, J=1.6 Hz, 1H), 7.34 (s, 1H), 3.94 (s, 2H), 3.72 (s, 3H).

### General Procedure for Preparation of Compound 9 of FIG. 25:

To a solution of compound 8 (1 mol equivalent) in THF was added a solution of LiOH·H₂O (5 mol equivalents) in H₂O (2 mL) at 25° C. The mixture was stirred at 25° C. for 16 hours, while being monitored by LCMS. The pH of the mixture was thereafter adjusted to pH=3.0 with 1M HCl and the obtained solution was extracted with DCM. The combined organic layers were washed with brine, dried over Na₂SO₄, filtered and concentrated under reduced pressure to give compound 9 (100 mg, crude), which was used directly in next step.

Compound 9A was obtained using 8A as a starting material (crude).

LCMS: RT=0.584 minutes, M/Z=390.1 [M+H]⁺

Compound 9B was obtained using 8B as a starting material (crude).

Compound 9C was obtained using 8C as a starting material (crude).

LCMS: RT=0.571 minutes, M/Z=389.0 [M+H]⁺

¹H-NMR (400 MHz, METHANOL-d₄): δ (ppm)=8.64 (s, 1H), 8.62 (s, 1H), 8.04 (s, 1H), 7.97 (s, 1H), 7.69 (s, 1H), 7.39 (s, 1H), 3.83 (s, 2H).

Compound 9D was obtained using 8D as material (crude).

LCMS: RT=0.386 minutes, M/Z=389.0 [M+H]⁺

¹H-NMR (400 MHz, CHLOROFORM-d): δ (ppm)=8.70 (s, 1H), 8.61 (s, 1H), 7.78 (s, 1H), 7.65 (s, 1H), 7.29 (s, 1H), 7.10 (s, 1H), 3.79 (s, 2H).

### General Procedure for Preparation of Final Products of FIG. 25:

To a solution of compound 9 (1 mol equivalent), amine (6 mol equivalents) and DIEA (5 mol equivalents) in DCM was added T₃P (1.5 mol equivalent) at 25° C. The reaction mixture was stirred at 25° C. for 4 hours, while being monitored using LCMS. The reaction mixture was thereafter concentrated under reduced pressure to dryness to give a crude product, which was purified by reversed-phase HPLC.

Compound 762 was obtained using 9A and 2-aminoethan-1-ol as starting materials (43% yield).

LCMS: RT=0.534 minutes, M/Z=432.8 [M+H]⁺

HPLC: product: RT=2.515 minutes, purity=98.627%

¹⁹F-NMR (400 MHz DMSO-d₆): δ (ppm)=56.721

¹H-NMR (400 MHz DMSO-d6): δ (ppm)=9.02 (d, J=5.6 Hz, 1H), 8.57 (d, J=1.2 Hz, 1H), 8.16 (t, J=5.6 Hz, 1H), 7.81 (d, J=5.2 Hz, 1H), 7.77 (d, J=0.4 Hz, 1H), 7.66 (d, J=2.0 Hz, 1H), 4.70 (s, 1H), 3.85 (s, 2H), 3.42 (t, J=6.0 Hz, 2H), 3.15 (q, J=5.6 Hz, 11.6 Hz, 2H)

Compound 763 was obtained using 9A and (3R,4S)-4-aminotetrahydrofuran-3-ol as starting materials (41% yield).

LCMS: RT=0.514 minutes, M/Z=475.1 [M+H]⁺

HPLC: RT=2.499 minutes, purity=99.369%

SFC: RT=1.719 minutes, ee=100%

¹⁹F-NMR (400 MHz DMSO-d6): δ (ppm)=56.721

¹H-NMR (400 MHz DMSO-d6): δ (ppm)=9.02 (d, J=5.2 Hz, 1H), 8.56 (d, J=1.2 Hz, 1H), 8.36 (d, J=6.8 Hz, 1H), 7.81 (d, J=5.2 Hz, 1H), 7.77 (s, 1H), 7.66 (d, J=1.6 Hz, 1H), 5.25 (d, J=3.6 Hz, 1H), 4.06-4.05 (m, 1H), 4.01-3.98 (m, 1H), 3.92-3.81 (m, 4H), 3.54-3.48 (m, 2H)

Compound 843 was obtained using 9B and 2-aminoethan-1-ol as starting materials (21% yield).

¹H-NMR (400 MHz, CDCl₃): δ (ppm)=9.14 (s, 1H), 7.98 (s, 1H), 7.73 (s, 1H), 7.43 (s, 1H), 7.38 (s, 1H), 7.07 (br s, 1H), 3.84 (s, 2H), 3.77 (t, J=4.8 Hz, 2H), 3.49 (q, J=4.8, 10.0 Hz, 2H).

LCMS: RT=0.541 minutes, M/Z=433.1 [M+H]⁺

HPLC: RT=2.437 minutes, purity=98.496%

Compound 844 was obtained using 9B and (3R,4S)-4-aminotetrahydrofuran-3-ol as starting materials (56% yield).

¹H-NMR (400 MHz, CDCl₃): δ (ppm)=9.14 (s, 1H), 7.99 (s, 1H), 7.74 (s, 1H), 7.44 (s, 1H), 7.35 (s, 1H), 7.17 (br s, 1H), 4.35-4.27 (m, 1H), 4.23-4.17 (m, 1H), 4.17-4.05 (m, 2H), 3.83 (s, 2H), 3.77-3.66 (m, 2H).

¹⁹F-NMR (400 MHz, CDCl₃): δ (ppm)=−58.720

LCMS: RT=0.541 minutes, M/Z=475.1 [M+H]⁺

HPLC: RT=1.704 minutes, purity=96.943%

SFC: de=96.620%

Compound 770 was obtained using 9C and (3R,4S)-4-aminotetrahydrofuran-3-ol as starting materials (47% yield).

LCMS: RT=0.603 minutes, M/Z=474.2 [M+H]⁺

HPLC: RT=1.975 minutes, purity=99.17%

¹H-NMR (400 MHz, CHLOROFORM-d): δ (ppm)=8.66 (s, 1H), 8.65 (s, 1H), 7.78 (s, 1H), 7.75-7.70 (m, 1H), 7.59-7.55 (m, 1H), 7.32 (d, J=2.0 Hz, 1H), 6.04-5.95 (m, 1H), 4.30-4.22 (m, 1H), 4.20-4.14 (m, 1H), 4.09 (d, J=5.2 Hz, 1H), 4.07 (d, J=5.2 Hz, 1H), 3.72-3.64 (m, 4H).

Compound 770_03 was obtained using 9D and (3R,4S)-4-aminotetrahydrofuran-3-ol as starting materials (67% yield).

LCMS: RT=0.583 minutes, M/Z=474.1 [M+H]⁺

HPLC: RT=1.873 minutes, purity=98.45%

¹H-NMR (400 MHz, CHLOROFORM-d): δ (ppm)=8.70-8.65 (m, 1H), 8.65-8.59 (m, 1H), 7.76 (s, 1H), 7.66 (d, J=1.6 Hz, 1H), 7.31 (d, J=2.0 Hz, 1H), 7.11 (s, 1H), 5.81 (br s, 1H), 4.25-4.14 (m, 2H), 4.10-3.98 (m, 2H), 3.70-3.58 (m, 4H), 3.22 (br s, 1H).

### Synthesis of Compounds 740, 742, 750, 756, 757, 760, 767, 766, 810, 820, 821, 822, 830, 832, 840, 842, 850, and 851:

Compounds 740, 742, 750, 756, 757, 760, 767, 766, 810, 820, 821, 822, 830, 832, 840, 842, 850, and 851 were prepared generally through conjugation of acid precursors, as presented in FIG. 19, with amine precursors, as presented in FIG. 20, in a synthetic protocol as presented in FIG. 26.

The following are exemplary synthetic protocols.

### Procedure for Preparation of Compound 3 of FIG. 26

To a solution of 5,7-dichloro-7,7a-dihydro-1H-indole (1.0 gram, 5.4 mmol, 1.0 mol equivalent) in dioxane (10 mL) was added dichlorohydantoin (600 mg, 3.0 mmol, 0.6 mol equivalent). The mixture was stirred at 25° C. for 3 hours, while being monitored using HPLC and LCMS. The reaction mixture was thereafter concentrated under reduced pressure to give a crude residue which was purified by column chromatography (SiO₂, Petroleum ether/Ethyl acetate=1/0) to afford compound 3 (1.5 grams, crude) as a white solid.

¹H-NMR (400 MHz CDCl₃): δ (ppm)=8.42 (br s, 1H), 7.60 (s, 1H), 7.38-7.30 (m, 2H).

### Procedure for Preparation of Compound 4A of FIG. 26

To a solution of 5-chloropyridazin-3(2H)-one (500 mg, 3.8 mmol, 1.0 mol equivalent) and pyridine (352 mg, 4.4 mmol, 358 μL, 1.2 mol equivalent) in MeCN (7 mL) was added TFAA (1.2 grams, 4.2 mmol, 695 μL, 1.1 mol equivalent) at 0° C. The reaction was warmed to 25° C. and stirred at 25° C. for 0.5 hours. NaI (2.8 grams, 19.2 mmol, 5.0 mol equivalent) and TFA (632 mg, 4.2 mmol, 373 μL, 1.1 mol equivalent) was added to the reaction. The reaction was stirred at 25° C. for 0.5 hours. TLC (PE:EA=5:1) showed two new spots were formed. The reaction was quenched with 10 mL (H₂O) and pH of the mixture was adjusted to 10.0 with NaOH solution (1 M). Then a solution of Na₂CO₃ (10%, 10 mL) and saturated Na₂SO₃ (25 mL) were added, and the obtained solution was extracted with EA (40 mL×3). The combined organic layer was washed with brine, dried over Na₂SO₄, filtered and concentrated under reduced pressure to dryness to give a crude product, which was purified by silica gel column chromatography (Petroleum ether/Ethyl acetate=100:1 to 5:1) to give 3.5 grams of a mixture of compounds 2 and 2a. The mixture was washed with MeOH (20 mL×3) (most of compound 2 was dissolved in MeOH). The filtrate was concentrated under reduced pressure to give compound 4A as a yellow solid (19.76% yield; 1.3 grams, 3.78 mmol, 70% purity)

LCMS: RT=0.410 minutes, M/Z=240.6 [M+H]⁺

### Procedure for Preparation of Compound 4B of FIG. 26

1,2,4-Triazine-3,5-diol (1.0 grams, 8.8 mmol, 1.0 mol equivalent) was added to POCl₃ (16.5 grams, 107.3 mmol, 10 mL, 12.0 mol equivalent) at 25° C. The mixture was stirred at 90° C. for 30 minutes under microwave, while being monitored by LCMS (quenched with NH₃/MeOH). The reaction mixture was thereafter extracted with hexane (50 mL), and the organic solution was filtered through celite, dried over Na₂SO₄ and concentrated under reduced pressure to dryness to give crude Compound 2 (5.5 grams) as a yellow solid which was further used directly.

### Procedure for Preparation of Compound 4C of FIG. 26

To a solution of 4-chloropyrimidin-2-amine (15.0 grams, 115.8 mmol, 1.0 mol equivalent) and diiodomethane (155.0 grams, 579.0 mmol, 46.7 mL, 5.0 mol equivalents) in CH₃CN (30 mL) was added t-BuONO (47.7 grams, 463.2 mmol, 55.0 mL, 4.0 mol equivalents). The reaction mixture was stirred at 80° C. for 3 hours, while being monitored using TLC (PE:EA=5:1). The reaction mixture was thereafter concentrated under reduced pressure to give a crude residue, which was purified by column chromatography (SiO₂, Petroleum ether/Ethyl acetate=I/O to 5/1) to give compound 4C as a white solid (50.56% yield; 15.3 grams, 58.6 mmol, 92% purity).

LCMS: RT=0.459 minutes, M/Z=240.8 [M+H]⁺

### General Procedure for Preparation of Compound 5 of FIG. 26

Substitution method A: To a solution of compound 3 (1.0 mol equivalent) and base (2.0 mol equivalents) in a solvent was added compound 4 (1.5 mol equivalent). The mixture was stirred at an appropriate temperature for 3 to about 16 hours, while being monitored by TLC. The mixture was thereafter diluted with brine and extracted with EA. The combined organic layers were concentrated under reduced pressure and purified by column chromatography to afford compound 5.

Coupling method B: To a solution of compound 3 (1.0 mol equivalent) and compound 4 (0.8 to about 1.0 mol equivalent) in dioxane was added a base (2.0 mol equivalents), a ligand (0.15 to about 0.8 mol equivalent) and CuI (0.15 to about 0.4 mol equivalent). The mixture was stirred at an appropriate temperature for 16 hours under N₂, while being monitored by TLC.

Compound 5A was obtained following coupling method B using compound 4A (1.0 mol equivalent) as a starting material, CuI (0.4 mol equivalent) as a catalyst, L-DMEDA (0.8 mol equivalent) as a ligand and K₂CO₃ as a base in a dioxane solution (120° C., 16 hours, purified by column chromatography, 11.4% yield).

LCMS: RT=0.658 minutes, M/Z=334.1 [M+H]⁺

Compound 5B was obtained following substitution method A, using 2,6-dichloropyrazine as a starting material, and K₂CO₃ as a base in DMF solution (80° C., 16 hours).

¹H-NMR (400 MHz, CDCl₃): δ (ppm)=8.55 (s, 1H), 8.50 (s, 1H), 7.55 (d, J=2.0 Hz, 1H), 7.44 (s, 1H), 7.28 (d, J=2.0 Hz, 1H), 7.19 (s, 1H).

Compound 5C was obtained following substitution method A using 2,4-dichloropyrimidine as a starting material, and K₂CO₃ as a base in DMF solution (80° C., 5 hours 75.88% yield). LCMS: RT=0.712 minutes, M/Z=334.1 [M+H]⁺

Compound 5D was obtained following substitution method A using 4B as a starting material, and Cs₂CO₃ as a base in MeCN solution (80° C., 1 hour by a short column to remove the inorganic salt, crude product used directly).

Compound 5E was obtained following coupling method B using compound 4C (0.8 mol equivalent) as a starting material, CuI (0.15 mol equivalent), L-Proline (0.15 mol equivalent) as a ligand and Cs₂CO₃ as a base in dioxane solution (70° C., 16 hours, crude). The reaction mixture was used in the next step directly.

Compound 5F was obtained following substitution method A using 3,5-dichloropyridazine as a starting material, K₂CO₃ as a base in DMF solution (80° C., 16 hours 54% yield).

¹H-NMR (400 MHz, d-DMSO): δ (ppm)=9.58 (d, J=2.0 Hz, 1H), 8.35 (d, J=2.0 Hz, 1H), 8.17 (s, 1H), 7.74 (d, J=1.6 Hz, 1H), 7.63 (d, J=1.6 Hz, 1H).

LCMS: RT=0.640 minutes, M/Z=333.9 [M+H]⁺

Compound 5G was obtained following substitution method A using 4,6-dichloropyrimidine as a starting material, and K₂CO₃ as a base in DMF solution (70° C., 16 hours, 42.3% yield).

¹H-NMR (400 MHz DMSO): δ (ppm)=9.10 (s, 1H), 8.20 (s, 1H), 8.09 (s, 1H), 7.71 (d, J=1.6 Hz, 1H), 7.63 (d, J=1.6 Hz, 1H).

Compound 5H was obtained following substitution method A using 2,4-dichloro-1,3,5-triazine as a starting material, and Cs₂CO₃ as a base in MeCN solution (25° C., 3 hours, purified by a short column chromatography to remove the inorganic salt). The crude product was used directly in the next step.

### General Procedure for Preparation of Compound 6 of FIG. 26:

To a solution of compound 5 (1.0 mol equivalent) and a base (2.0 mol equivalent) in a solvent was added dialkyl propanedioate (1.5 mol equivalent). The mixture was stirred at an appropriate temperature for 1 to about 16 hours, while being monitored by TLC or LCMS. The mixture was thereafter quenched with water and extracted with EA. The organic layer was concentrated under reduced pressure and the obtained residue was purified by column chromatography or trituration to give compound 6.

Compound 6A was obtained using compound 5A, dimethyl propanedioate, and Cs₂CO₃ as a base in DMF solution (100° C., 16 hours 96% yield).

Compound 6B was obtained using compound 5B, dimethyl propanedioate, and Cs₂CO₃ as a base in DMF solution (100° C., 16 hours 17.25% yield).

LCMS: RT=0.657 minutes, M/Z=428.0 [M+H]⁺

Compound 6C was obtained using compound 5C, dimethyl propanedioate, and Cs₂CO₃ as a base in DMF solution (100° C., 16 hours 80.32% yield).

LCMS: RT=0.644 min, M/Z=429.7 [M+H]⁺

Compound 6D was obtained using compound 5D, di-tert-butyl propanedioate, and Cs₂CO₃ as a base in MeCN solution (80° C., 2 hours 17% yield).

Compound 6E was obtained using compound 5E and dimethyl propanedioate. To the reaction mixture of compound 5E was added dimethyl propanedioate and it was stirred at 100° C. for 5 hours. Purification by column chromatography gave compound 6E (14.60% yield).

HPLC: RT=3.836 minutes, purity=80.075%

LCMS: RT=0.825 minutes, M/Z=429.9 [M+H]⁺

Compound 6F was obtained using compound 5F, dimethyl propanedioate, and Cs₂CO₃ as a base in DMF solution (100° C., 16 hours 43% yield).

¹H-NMR (400 MHz, d-DMSO): δ (ppm)=9.57 (d, J=2.8 Hz, 1H), 8.17 (s, 1H), 7.99 (d, J=2.0 Hz, 1H), 7.72 (d, J=2.0 Hz, 1H), 7.59 (d, J=2.0 Hz, 1H), 5.55 (s, 1H), 3.73 (s, 6H).

Compound 6G was obtained using compound 5G, tert-butyl ethyl malonate, and Cs₂CO₃ as a base in DMF solution (100° C., 3 hours 85.45% yield).

¹H-NMR (EW42593-1-P1B1, 400 MHz CDCl₃): δ (ppm)=9.11 (s, 1H), 7.70 (s, 1H), 7.62 (d, J=2.0 Hz, 1H), 7.49 (d, J=0.8 Hz, 1H), 7.38 (s, 1H), 4.88 (s, 1H), 4.31-4.25 (m, 2H), 1.51 (s, 9H), 1.31 (t, J=7.2 Hz, 3H).

Compound 6H was obtained using compound 5H, di-tert-butyl propanedioate, and NaH as a base in THF solution (0 to about 25° C., 1 hours 74% yield).

### General Procedure for Preparation of Compound 7 of FIG. 26

Method A: To a solution of compound 6 (1.0 mol equivalent) in DMSO/H₂O (5:1) was added LiCl (1.5 mol equivalent). The mixture was stirred at an appropriate temperature for 3 to about 36 hours, while being monitored by LCMS. The mixture was thereafter diluted with H₂O and extracted with EA. The combined organic layers were washed with brine, dried over Na₂SO₄, filtered and concentrated under reduced pressure, or purified by NPLC/silica gel column chromatography to give compound 7.

Method B: To a solution of compound 6 (1 mol equivalent) in DCM was added TFA (32.63 mol equivalents) at 0° C. The mixture was stirred at 25° C. for 1 hour, while being monitored by TLC. The mixture was thereafter concentrated under reduced pressure to remove the solvent. The remaining residue was diluted with EA and washed with NaHCO₃, dried over Na₂SO₄, filtered and concentrated under reduced pressure to give a crude product, which was purified by column chromatography to afford compound 7.

Compound 7A was obtained following method A using compound 6A as a starting material (100° C., 16 hours 42.3% yield) and was purified by preparative NPLC.

HPLC: RT=3.154 minutes, purity=81.805%

Compound 7B was obtained following method A using compound 6B as a starting material and was purified by extraction and concentration (130° C., 16 hours 42.3% yield).

Compound 7C was obtained following method A using compound 6C as a starting material and was purified by silica gel column chromatography (100° C., 36 hours 44.3% yield).

LCMS: RT=0.653 minutes, M/Z=371.6 [M+H]⁺

Compound 7E was obtained following method A using compound 6E as a starting material and was purified by silica gel column chromatography (90° C., 5 hours 40% yield).

LCMS: RT=0.664 minutes, M/Z=370.0 [M+H]⁺

Compound 7F was obtained following method A using compound 6F as a starting material and was purified by reversed-phase HPLC (100° C., 3 hours 42% yield).

Compound 7G was obtained following method B using compound 6G as a starting material (94% yield).

¹H-NMR (EW42593-9-P1B1, 400 MHz CDCl3): δ (ppm)=9.10 (s, 1H), 7.70 (s, 1H), 7.62 (d, J=1.6 Hz, 1H), 7.38 (d, J=2.0 Hz, 1H), 7.29 (s, 1H), 4.23 (q, J=7.2 Hz, 2H), 3.91 (s, 2H), 1.29 (t, J=7.2 Hz, 3H).

### General Procedure for Preparation of Compound 8. of FIG. 26

Method A: To a solution of compound 7 (1.0 mol equivalent) in a solvent was added a base (3 to about 5 mol equivalents) in H₂O. The mixture was stirred at an appropriate temperature for 3 to about 36 hours, while being monitored by LCMS. The mixture was thereafter diluted with H₂O, and acidified by HCl (1 μM) to pH=3, and then was extracted with EA (5 mL×3). The combined organic layers were washed with brine (5 mL×3), dried over Na₂SO₄, filtered and concentrated under reduced pressure to give compound 8.

Method B: To a solution of compound 6 (1.0 mol equivalent) in DCM was added TFA (20.7 mol equivalents) at 0° C. and the reaction mixture was stirred at 0° C. for 0.5 hours. Then it was warmed to 20° C. and stirred at 20° C. for 0.5 hour, while being monitored by LCMS. The mixture was thereafter concentrated under reduced pressure to give compound 8.

Compound 8A was obtained following method A using compound 7A as a starting material, LiOH·H₂O (5.0 mol equivalents) as a base in THF solution (20° C., 2 hours crude).

Compound 8B was obtained following method A using compound 7B as a starting material, LiOH·H₂O (5.0 mol equivalents) as a base in THF solution (25° C., 2 hours crude).

Compound 8C was obtained following method A using compound 7C as a starting material, LiOH·H₂O (5.0 mol equivalents) as a base in THF solution (20° C., 2 hours crude).

Compound 8D was obtained following method B using compound 6D as a starting material (0 to about 25° C., 1 hours crude).

Compound 8E was obtained following method A using compound 7E as a starting material, LiOH·H₂O (5.0 mol equivalents) as a base in THF solution (25° C., 2 hours crude).

LCMS: RT=0.612 min, M/Z=358.1 [M+H]⁺

Compound 8F was obtained following method A using compound 7F as a starting material, NaOEt (3.0 mol equivalents) as a base in EtOH solution (25 to about 35° C., 1 hours crude).

Compound 8G was obtained following method A using compound 7G as a starting material, LiOH·H₂O (5.0 mol equivalents) as a base in THF solution (25° C., 4 hours crude).

LCMS: RT=0.592 minutes, M/Z=358.1 [M+H]⁺

Compound 8H was obtained following method B using compound 6H as a starting material (0 to about 25° C., 1 hour, crude).

### General Procedure for Preparation of Final Products of FIG. 26

Method A: To a solution of compound 8 (1.0 mol equivalent), amine (2.5 mol equivalents) and DIEA (5.0 mol equivalents) in DCM was added T₃P (3.0 mol equivalents) at 0° C. The reaction was stirred at 25° C. for 1 hour, while being monitored by LCMS. The mixture was thereafter diluted with H₂O and extracted with EA (3 times). The combined organic layers were washed with brine, dried over Na₂SO₄, filtered and concentrated under reduced pressure to give a crude product, which was purified by preparative HPLC.

Method B: To a solution of compound 8 (1.0 mol equivalent) and DIEA (2.0 mol equivalents) in DMF (1 mL) was added HATU (1.2 mol equivalent) at 0° C. The mixture was stirred at 0° C. for 0.5 hours. Then an amine (1.5 mol equivalent) was added, and the reaction mixture was stirred at 15° C. for 0.5 hour, while being monitored by LCMS. The mixture was thereafter concentrated under reduced pressure and purified by preparative HPLC.

Method C: To a solution of compound 7 (1.0 mol equivalent) in EtOH was added an amine precursor (10 mol equivalents) and NaOEt (3.0 mol equivalents) at 25° C. The mixture was stirred at 35° C. for 6 hours, while being monitored by LCMS. The mixture was thereafter poured into ice water and extracted with DCM. The combined organic layers were washed with brine, dried over Na₂SO₄, filtered and concentrated under reduced pressure to give a residue, which was purified by preparative HPLC.

Compound 740 was obtained following method A using compound 8′ and (3S,4R)-4-aminotetrahydrofuran-3-ol as an amine precursor (23.65% yield).

¹H-NMR (400 MHz, d-DMSO): δ (ppm)=9.28 (s, 1H), 8.48 (d, J=6.8 Hz, 1H), 8.17 (s, 1H), 7.90 (s, 1H), 7.73 (d, J=2.0 Hz, 1H), 7.55 (d, J=2.0 Hz, 1H), 5.29 (d, J=3.6 Hz, 1H), 4.10-3.80 (m, 4H), 3.70 (s, 2H), 3.58-3.48 (m, 2H).

SFC: ee=100%

Compound 742 was obtained following method A using compound 8A and 2-aminoethan-1-ol as an amine precursor (56% yield).

¹H-NMR (400 MHz, d-DMSO): δ (ppm)=9.28 (s, 1H), 8.29 (t, J=5.2 Hz, 1H), 8.17 (s, 1H), 7.91 (s, 1H), 7.72 (d, J=2.0 Hz, 1H), 7.55 (d, J=2.0 Hz, 1H), 4.72 (s, 1H), 3.69 (s, 2H), 3.48-3.38 (m, 2H), 3.20-3.10 (m, 2H).

LCMS: RT=0.500 minutes, M/Z=400.7 [M+H]⁺

HPLC: RT=2.239 minutes, purity=98.460%

Compound 750 was obtained following method B using compound 8B and (3S,4R)-4-aminotetrahydrofuran-3-ol as an amine precursor (26% yield).

¹H-NMR (400 MHz, CDCl₃): δ (ppm)=8.66 (d, J=5.2 Hz, 1H), 7.65 (d, J=1.6 Hz, 1H), 7.44 (s, 1H), 7.34 (d, J=2.0 Hz, 1H), 6.55 (br s, 1H), 4.29-4.24 (m, 1H), 4.21-4.14 (m, 1H), 4.11-4.05 (m, 1H), 4.03-3.97 (m, 1H), 3.83 (s, 2H), 3.68-3.58 (m, 2H).

LCMS: RT=0.525 minutes, M/Z=442.9 [M+H]⁺

HPLC: RT=1.539 minutes, purity=98.613%

SFC: de=98.522%

Compound 756 was obtained following method B using compound 8B and 3-(aminomethyl)oxetan-3-ol as an amine precursor (31% yield).

¹H-NMR (400 MHz, d-DMSO): δ (ppm)=8.86 (s, 1H), 8.76 (s, 1H), 8.12 (s, 1H), 7.69 (s, 1H), 7.52 (s, 1H), 5.86 (s, 1H), 4.34 (s, 4H), 3.84 (s, 2H), 3.40 (s, 2H).

LCMS: RT=0.523 minutes, M/Z=441.2 [M+H]⁺

Compound 757 was obtained following method B using compound 8B and 2-aminoethan-1-ol as an amine precursor (20% yield).

¹H-NMR (400 MHz, d-DMSO): δ (ppm)=8.86 (s, 1H), 8.75 (s, 1H), 8.13 (s, 1H), 7.69 (d, J=1.6 Hz, 1H), 7.52 (d, J=1.6 Hz, 1H), 3.77 (s, 2H), 3.40 (t, J=6.0 Hz, 2H), 3.14 (t, J=6.0 Hz, 2H).

LCMS: RT=0.529 minutes, M/Z=401.2 [M+H]⁺

HPLC: RT=2.448 minutes, purity=98.005%

Compound 760 was obtained following method A using compound 8C and (3S,4R)-4-aminotetrahydrofuran-3-ol as an amine precursor (57% yield).

LCMS: RT=0.511 minutes, M/Z=443.1 [M+H]⁺

HPLC: product: RT=2.366 minutes, purity=98.719%

SFC: product: RT=1.413 minutes, ee=100%

¹H-NMR (400 MHz DMSO-d6): δ (ppm)=8.95 (d, J=5.6 Hz, 1H), 8.36 (d, J=6.4 Hz, 1H), 8.16 (s, 1H), 7.71 (d, J=2.0 Hz, 1H), 7.67 (d, J=5.2 Hz, 1H), 7.60 (d, J=5.2 Hz, 1H), 5.26 (d, J=4.0 Hz, 1H), 4.07-4.05 (m, 1H), 4.01-3.98 (m, 1H), 3.92-3.88 (m, 1H), 3.85-3.81 (m, 3H), 3.54-3.48 (m, 2H)

Compound 767 was obtained following method A using compound 8C and 2-aminoethan-1-ol as an amine precursor (33% yield).

LCMS: RT=0.514 minutes, M/Z=399.2 [M+H]⁺

HPLC: RT=2.377 minutes, purity=97.119%

¹H-NMR (400 MHz, DMSO-d6): δ (ppm)=8.93 (d, J=6.4 Hz, 1H), 8.16 (s, 1H), 8.14-8.11 (m, 1H), 7.70 (d, J=2.0 Hz, 1H), 7.66 (d, J=5.2 Hz, 1H), 7.59 (d, J=2.0 Hz, 1H), 4.69-4.66 (m, 1H), 3.82 (s, 2H), 3.42-3.41 (m, 2H), 3.17-3.12 (m, 2H)

Compound 766 was obtained following method A using compound 8C and 3-(aminomethyl)oxetan-3-ol as an amine precursor (36% yield).

LCMS: RT=0.549 minutes, M/Z=441.1 [M+H]⁺

HPLC: RT=1.815 minutes, purity=98.397%

¹H-NMR (400 MHz, DMSO-d6): δ (ppm)=8.94 (d, J=5.6 Hz, 1H), 8.28 (t, J=5.6 Hz, 1H), 8.14 (s, 1H), 7.70 (d, J=2.0 Hz, 1H), 7.65 (d, J=5.2 Hz, 1H), 7.58 (d, J=2.0 Hz, 1H), 5.85 (s, 1H), 4.40-4.30 (m, 4H), 3.88 (s, 2H), 3.40 (d, J=6.0 Hz, 1H).

Compound 810 was obtained following method A using compound 8D and (3S,4R)-4-aminotetrahydrofuran-3-ol as an amine precursor (8% yield).

¹H-NMR (400 MHz, d-DMSO): δ (ppm)=9.51 (s, 1H), 8.54 (d, J=6.8 Hz, 1H), 8.25 (s, 1H), 7.74 (d, J=2.0 Hz, 1H), 7.62 (d, J=2.0 Hz, 1H), 5.29 (d, J=3.6 Hz, 1H), 4.07-3.95 (m, 4H), 3.93-3.82 (m, 4H), 3.58-3.48 (m, 2H).

LCMS: RT=0.519 minutes, M/Z=444.1 [M+H]⁺

HPLC: RT=2.412 minutes, purity=99.205%

SFC: ee=100%

Compound 820 was obtained following method A using compound 8E and (3S,4R)-4-aminotetrahydrofuran-3-ol as an amine precursor (8% yield).

LCMS: RT=0.528 minutes, M/Z=441.7.0 [M+H]⁺

HPLC: RT=2.512 minutes, purity=100.00%

SFC: RT=1.146 minutes, ee=96.38%

¹H-NMR (400 MHz d-DMSO): δ (ppm)=8.87 (d, J=4.8 Hz, 1H), 8.43 (d, J=6.8 Hz, 1H), 8.14 (s, 1H), 7.68 (d, J=2.0 Hz, 1H), 7.56-7.53 (m, 2H), 5.26 (d, J=3.6 Hz, 1H), 4.04-4.97 (m, 2H), 3.91-3.88 (m, 1H), 3.84-3.81 (m, 1H), 3.75 (s, 2H), 3.53-3.48 (m. 2H)

Compound 821 was obtained following method A using compound 8E and 2-aminoethan-1-ol as an amine precursor (23% yield).

LCMS: RT=0.533 minutes, M/Z=401.0 [M+H]⁺

HPLC: RT=2.491 minutes, purity=99.168%

¹H-NMR (400 MHz d-DMSO): δ (ppm)=8.87 (d, J=5.2 Hz, 1H), 8.23 (s, 1H), 8.14 (s, 1H), 7.68 (s, 1H), 7.55-7.50 (m, 2H), 4.67 (t, J=5.6 Hz, 1H), 3.74 (s, 2H), 3.50-3.40 (m, 2H), 3.20-3.08 (m, 2H).

Compound 822 was obtained following method A using compound 8E and 3-(aminomethyl)oxetan-3-ol as an amine precursor (46% yield).

LCMS: RT=0.521 minutes, M/Z=441.1 [M+H]⁺

HPLC: RT=2.521 minutes, purity=98.950%

¹H-NMR (400 MHz d-DMSO): δ (ppm)=8.87 (d, J=4.8 Hz, 1H), 8.39 (s, 1H), 8.13 (s, 1H), 7.68 (s, 1H), 7.55-7.50 (m, 2H), 5.87 (s, 1H), 4.35 (s, 4H), 3.81 (s, 2H), 3.40 (d, J=6.0 Hz, 2H).

Compound 830 was obtained following method A using compound 8F and (3S,4R)-4-aminotetrahydrofuran-3-ol as an amine precursor (52% yield).

¹H-NMR (400 MHz, d-DMSO): δ (ppm)=9.43 (d, J=2.0 Hz, 1H), 8.49 (d, J=2.8 Hz, 1H), 8.12 (s, 1H), 7.85 (d, J=2.0 Hz, 1H), 7.72 (d, J=1.2 Hz, 1H), 7.57 (d, J=1.2 Hz, 1H), 5.24 (s, 1H), 4.10-3.97 (m, 2H), 3.94 (s, 2H), 3.92-3.82 (m, 2H), 3.60-3.47 (m, 2H).

LCMS: RT=0.511 minutes, M/Z=441.2 [M+H]⁺

HPLC: RT=1.504 minutes, purity=98.223%

SFC: ee=96.664%

Compound 832 was obtained following method B using compound 7F and 2-aminoethan-1-ol as an amine precursor (52% yield).

¹H-NMR (400 MHz, d-DMSO): δ (ppm)=9.43 (d, J=2.0 Hz, 1H), 8.29 (t, J=5.2 Hz, 1H), 8.12 (s, 1H), 7.85 (d, J=2.0 Hz, 1H), 7.72 (s, 1H), 7.57 (s, 1H), 4.70 (t, J=5.2 Hz, 1H), 3.94 (s, 2H), 3.47-3.40 (m, 2H), 3.20-3.13 (m, 2H).

LCMS: RT=0.506 minutes, M/Z=401.2 [M+H]⁺

HPLC: RT=1.502 minutes, purity=97.981%

Compound 840 was obtained following method A using compound 8G and (3S,4R)-4-aminotetrahydrofuran-3-ol as an amine precursor (47% yield).

LCMS: EW42593-16-P1E1, RT=0.532 minutes, M/Z=443.2 [M+H]⁺

HPLC: EW42593-16-P1E2, product: RT=1.596 minutes, purity=99.770%

SFC: EW42593-16-P1D1, RT=0.235 minutes, ee %=94.996%

¹H-NMR (EW42593-16-P1E1, 400 MHz DMSO): δ (ppm)=9.12 (s, 1H), 8.44 (d, J=6.8 Hz, 1H) 8.16 (s, 1H), 7.71 (d, J=1.6 Hz, 1H), 7.67 (s, 1H), 7.61 (s, 1H), 5.27 (s, 1H), 4.06 (brs, 1H), 3.99-3.92 (m, 1H), 3.90-3.93 (m, 2H), 3.77 (s, 2H), 3.52 (t, J=11.2 Hz, 2H).

Compound 842 was obtained following method A using compound 8G and 2-aminoethan-1-ol as an amine precursor (23% yield).

LCMS: EW42593-21-P1D1, RT=0.535 minutes, M/Z=399.1 [M+H]⁺

HPLC: EW42593-21-P1E4, product: RT=1.872 minutes, purity=97.872%

¹H-NMR (EW42593-21-P1D1, 400 MHz DMSO): δ (ppm)=9.12 (s, 1H), 8.25-8.23 (m, 1H), 8.15 (s, 1H), 7.71 (d, J=1.6 Hz, 1H) 7.68 (s, 1H), 7.60 (d, J=1.6 Hz, 1H), 3.76 (s, 2H), 3.44-3.40 (m, 2H), 3.18-3.13 (m, 2H).

Compound 850 was obtained following method A using compound 8H and (3S,4R)-4-aminotetrahydrofuran-3-ol as an amine precursor (1.3% yield).

¹H-NMR (400 MHz, CDCl₃): δ (ppm)=9.10 (s, 1H), 8.49 (d, J=2.8 Hz, 1H), 7.99 (s, 1H), 7.59 (d, J=2.0 Hz, 1H), 7.47 (d, J=2.0 Hz, 1H), 7.20-7.10 (m, 1H), 4.35-4.27 (m, 1H), 4.24-4.17 (m, 1H), 4.15-4.02 (m, 2H), 3.95 (s, 2H), 3.72-3.66 (m, 2H), 2.92 (s, 1H).

LCMS: RT=0.537 minutes, M/Z=442.0 [M+H]⁺

HPLC: RT=1.634 minutes, purity=94.348%

Compound 850 was obtained following method A using compound 8H and 2-aminoethan-1-ol as an amine precursor (9.8% yield).

¹H-NMR (400 MHz, CDCl₃): δ (ppm)=9.10 (s, 1H), 8.00 (s, 1H), 7.58 (d, J=2.0 Hz, 1H), 7.46 (d, J=2.0 Hz, 1H), 7.20 (s, 1H), 3.96 (s, 2H), 3.82-3.75 (m, 2H), 3.55-3.46 (m, 2H).

LCMS: RT=0.516 minutes, M/Z=400.0 [M+H]⁺

HPLC: RT=1.634 minutes, purity=94.348%

### Synthesis of Compounds 760 and 767

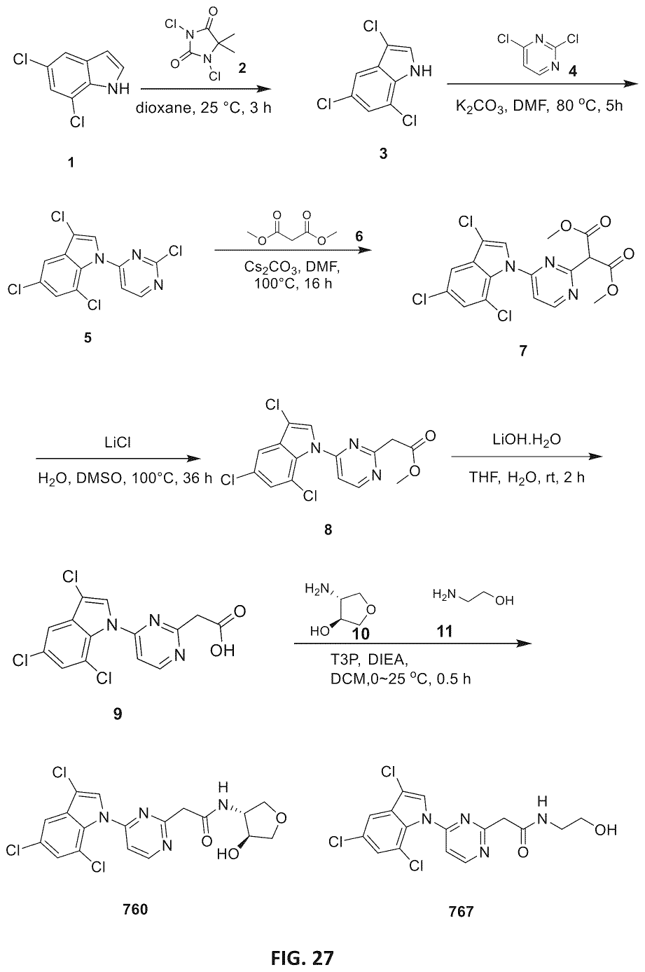

Compounds 760 and 767 were prepared generally through conjugation of acid precursors, as presented in FIG. 19, with amine precursors, as presented in FIG. 20, in a synthetic protocol as presented in FIG. 27.

The following are exemplary synthetic protocols.

### General Procedure for Preparation of Compound 3 of FIG. 27

To a solution of compound 1 (1 gram, 5.38 mmol, 1 mol equivalent) in dioxane (10 mL) was added compound 2 (600.47 mg, 3.05 mmol, 0.567 mol equivalent), and the mixture was stirred at 25° C. for 3 hours, while being monitored by HPLC and LCMS. The reaction mixture was thereafter concentrated under reduced pressure to give a crude product, which was purified by column chromatography (SiO₂, Petroleum ether/Ethyl acetate=1/0) to afford compound 3 (1.5 grams, crude) as a white solid.

¹H-NMR (400 MHz CDCl₃): δ (ppm)=8.52-8.27 (m, 1H), 7.60 (brs, 1H), 7.38-7.30 (m, 2H).

### General Procedure for Preparation of Compound 5 of FIG. 27

To a solution of compound 3 (6 grams, 27.21 mmol, 1 mol equivalent) and K₂CO₃ (7.52 grams, 54.43 mmol, 2 mol equivalents) in DMF (60 mL) was added compound 4 (4.46 grams, 29.93 mmol, 1.1 mol equivalent). The mixture was stirred at 80° C. for 5 hours, while being monitored by TLC (PE/EA=10/1, Rf=0.43). The mixture was thereafter diluted with ice water (180 mL), and extracted with EA (180 mL×3). The combined organic layer was washed with brine (100 mL×3), dried with Na₂SO₄, filtered and concentrated under reduced pressure to give a crude product, which was purified by silica gel column chromatography (SiO₂, Petroleum ether/Ethyl acetate=100/1 to 5/1) to give compound 5 (7.5 grams, 20.65 mmol, 75.88% yield, 91.68% purity) as white solid.

LCMS: RT=0.712 minutes, M/Z=334.1 [M+H]⁺

### General Procedure for Preparation of Compound 7 of FIG. 27

To a solution of compound 5 (6 grams, 18.02 mmol, 1 mol equivalent) and Cs₂CO₃ (11.74 grams, 36.04 mmol, 2 mol equivalents) in DMF (70 mL) was added compound 6 (3.57 grams, 27.03 mmol, 3.10 mL, 1.5 mol equivalent). The mixture was stirred at 100° C. for 16 hours, while being monitored by LCMS (EW40799-27-P1A). The reaction mixture was thereafter combined with EW40799-23 (scale: 1 g) and then poured into 250 mL of ice H₂O. The mixture was extracted with DCM (100 mL×3). The organic layer was washed with brine (50 mL×2), dried over Na₂SO₄, filtered and concentrated under reduced pressure to dryness to give a crude product, which was purified by trituration (PE:EA=5:1) to give compound 7 (7.2 grams, 16.80 mmol, 80.36% yield) as a brown solid. The product was used directly without further purification.

LCMS: RT=0.644 minutes, M/Z=429.7 [M+H]⁺

### General Procedure for Preparation of Compound 8 of FIG. 27

A solution of compound 7 (6.5 grams, 15.16 mmol, 1 mol equivalent) in DMSO (140 mL) and H₂O (4 mL) was added LiCl (1.29 grams, 30.33 mmol, 621.71 μL, 2 mol equivalents). The reaction was stirred at 100° C. for 36 hours, while being monitored by LCMS. The reaction was combined with EW40799-28 (scale: 0.5 g) and then poured into 250 mL of ice water. The mixture was extracted with EA (80 mL×4). The organic layer was washed with brine (50 mL×3), dried over Na₂SO₄, filtered and concentrated under reduced pressure to dryness to give a crude product, which was purified by silica gel column chromatography (SiO₂, Petroleum ether/Ethyl acetate=100/1 to 10/1) to give compound 8 (2.8 grams, 7.18 mmol, 44.23% yield, 95% purity) as a yellow solid.

LCMS: RT=0.653 min, M/Z=371.6 [M+H]⁺

### General Procedure for Preparation of Compound 9 of FIG. 27

To a solution of compound 8 (2.8 grams, 7.55 mmol, 1 mol equivalent) in THF (20 mL) was added a solution of LiOH·H₂O (1.59 grams, 37.77 mmol, 5 mol equivalents) in H₂O (10 mL). The mixture was stirred at 20° C. for 2 hours, while being monitored by LCMS. The mixture was thereafter diluted with ice water (30 mL), the pH was adjusted to pH=3 with 1M HCl, and the obtained mixture was extracted with EA (20 mL×3). The combined organic layer was washed with brine (30 mL*×3), dried over Na₂SO₄, filtered and concentrated under reduced pressure to give compound 9 (1.9 grams, 55% purity) as a light yellow solid MS. The crude product was used directly without further purification.

LCMS: RT=0.580 min, M/Z=356.2 [M+H]⁺

### Procedure for Preparation of Compound 760 (FIG. 27)

To a solution of compound 9 (150 mg, 420.65 μmol, 1 mol equivalent), amine 10 (65.07 mg, 630.98 μmol, 1.5 mol equivalent) and DIEA (271.82 mg, 2.10 mmol, 366.34 μL, 5 mol equivalents) in DCM (1.5 mL) was added T₃P (401.53 mg, 630.98 μmol, 375.61 μL, 50% purity, 1.5 mol equivalent) at 0° C. The reaction was slowly warmed to 25° C. and stirred at 25° C. for 0.5 hours, while being monitored by LCMS. The reaction was thereafter concentrated to give a crude product, which was purified by reversed-phase HPLC (0.1% NH₃·H₂O) to give compound 760 (138.4 mg, 313.34 μmol, 57.30% yield, 100% purity) as a white solid.

LCMS: RT=0.511 min, M/Z=443.1 [M+H]⁺

HPLC: product: RT=2.366 minutes, purity=98.719%

SFC: product: RT=1.413 minutes, ee=100%

¹H-NMR (400 MHz DMSO-d6): δ (ppm)=8.95 (d, J=5.6 Hz, 1H), 8.36 (d, J=6.4 Hz, 1H), 8.16 (s, 1H), 7.71 (d, J=2.0 Hz, 1H), 7.67 (d, J=5.2 Hz, 1H), 7.60 (d, J=5.2 Hz, 1H), 5.26 (d, J=4.0 Hz, 1H), 4.07-4.05 (m, 1H), 4.01-3.98 (m, 1H), 3.92-3.88 (m, 1H), 3.85-3.81 (m, 3H), 3.54-3.48 (m, 2H)

### Procedure for Preparation of Compound 767

To a solution of compound 9 (500.00 mg, 1.40 mmol, 1 mol equivalent), compound 11 (214.12 mg, 3.51 mmol, 2.5 mol equivalents) and DIEA (906.10 mg, 7.01 mmol, 1.22 mL, 5 mol equivalents) in DCM (10 mL) was added T₃P (1.34 grams, 2.10 mmol, 1.25 mL, 50% purity, 1.5 mol equivalent) at 0° C. The reaction was slowly warmed to 25° C., and stirred at 25° C. for 0.5 hours, while being monitored by LCMS. The reaction was thereafter concentrated to give a crude product, which was purified by reversed-phase HPLC (0.1% NH₃·H₂O) to give compound 767 (188 mg, 468.99 μmol, 33.45% yield, 99.7% purity) as a white solid.

LCMS: RT=0.514 minutes, M/Z=399.2 [M+H]⁺

HPLC: product: RT=2.377 minutes, purity=97.119%

¹H-NMR (400 MHz, DMSO-d6): δ (ppm)=8.93 (d, J=6.4 Hz, 1H), 8.16 (s, 1H), 8.14-8.11 (m, 1H), 7.70 (d, J=2.0 Hz, 1H), 7.66 (d, J=5.2 Hz, 1H), 7.59 (d, J=2.0 Hz, 1H), 4.69-4.66 (m, 1H), 3.82 (s, 2H), 3.42-3.41 (m, 2H), 3.17-3.12 (m, 2H)

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A compound represented by Formula I: 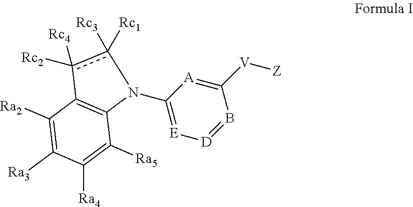
wherein:
A is C—Rb1 or N;
B is C—Rb2 or N;
D is C—Rb3 or N;
E is C—Rb4 or N;
Ra2-Ra5 are each independently hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of Ra2-Ra5 form together an alicyclic or aromatic ring;
Rb1, Rb2, Rb3 and Rb4 are each independently hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Rb substituents form together an alicyclic, heteroalicyclic, aromatic or heteroaromatic ring;
Rc1-Rc4 are each independently absent, hydrogen or a substituent selected from alkyl, cycloalkyl, halo, haloalkyl, haloalkoxy, alkoxy, ether, aryl, heteroaryl, heteroalicyclic, aryloxy, hydroxy, amine, alkylamine, thiohydroxy, thioalkoxy, thioaryloxy, cyano, carboxylate, amide, carbamate, sulfonyl, and sulfonamide, or, alternatively, at least two of the Rc substituents form together an alicyclic or heterocyclic ring,
the dashed line represents a single bond (when absent) or a double bond (when present), such that when the dashed line represents a double bond, Rc3 and Rc4 are absent;
V is —(CR₂R₃)k-U;
k is 0, 1 or 2;
R₂ and R₃ are each independently hydrogen, alkyl, cycloalkyl and aryl;
U is amide (—C(═O)—NR₁₀—) or an isostere thereof, and
Z is represented by Formula II: 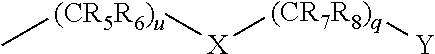
wherein:
u and q are each independently an integer of from 0 to 4, provided that u+q is at least 2;
X is selected from —O— and —NR₉—, or is absent;
Y is a polar hydrophilic group such as OR₁₁, SR₁₁, amine (NR₁₂R₁₃), or amide (—NR₁₂—C(═O)—R₁₄;
R₅, R₆, R₇ and R₈ are each independently selected from hydrogen, halo, alkyl, haloalkyl, cycloalkyl, heteroalicyclic, aryl, alkylamine, alkoxy, haloalkoxy, hydroxy, ether and aryloxy, or, alternatively, two of R₅, R₆, R₇, R₈ and R₉ form together an alicyclic or heteroalicyclic ring; and
R₉, R₁₀, R₁₁, R₁₂, R₁₃ and R₁₄ are each independently selected from hydrogen, alkyl, cycloalkyl, and aryl, or, alternatively, two of R₅, R₆, R₇, R₈, R₉ and R₁₁ or two of R₅, R₆, R₇, R₈, R₉, R₁₂ and R₁₃ form together an alicyclic or heteroalicyclic ring,
wherein at least one of A, B, D and E is N.

2. The compound of claim 1, wherein the dashed line represents a double bond.

3. The compound of claim 1, wherein at least two of A, B, D and E are each N.

4. The compound of claim 3 wherein at least one, or each, of A and B is N.

5. The compound of claim 3, wherein E and B are each N.

6. The compound of claim 3, wherein E, A and B are each N.

7. The compound of claim 1, wherein X is absent.

8. The compound of claim 1, wherein u is 1 or 2.

9. The compound of claim 1, wherein at least two of R₅, R₆, R₇ and R₈ form together an alicyclic ring or a heteroalicyclic ring.

10. The compound of claim 1, wherein X is absent, u is 1 or 2, and q is 1.

11. The compound of claim 1, wherein Y is OR₁₁, and Ru is hydrogen or an alkyl, wherein said alkyl is optionally substituted by at least one hydroxy, amide and/or carboxy or a heteroaryl or heteroalicyclic.

12. The compound of claim 1, selected from the compounds presented in FIG. 17.

13. The compound of claim 1, being selected from:

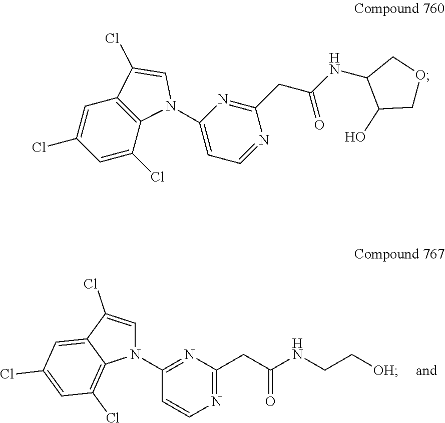 [SMILES: O=C(Cc1nccc(-n2cc(Cl)c3cc(Cl)cc(Cl)c32)n1)NC1COCC1O ; O=C(Cc1nccc(-n2cc(Cl)c3cc(Cl)cc(Cl)c32)n1)NCCO ; "and"]

14. A pharmaceutical composition comprising the compound of claim 1 and a pharmaceutically acceptable carrier.

15. A method of modulating an activity of a voltage-dependent potassium channel, the method comprising contacting the voltage-dependent potassium channel or a cell, a tissue or an organ which expresses the voltage-dependent potassium channel with the compound of claim 1, wherein when said contacting is effected in vivo, the method comprises administering to a subject in need thereof a therapeutically effective amount of the compound.

16. A method of modulating an activity of a TRPV1 channel, the method comprising contacting the TRPV1 channel or a cell, a tissue or an organ which expresses the TRPV1 channel with the compound of claim 1, wherein when said contacting is effected in vivo, the method comprises administering to a subject in need thereof a therapeutically effective amount of the compound.

17. A method of modulating an activity of both a voltage-dependent potassium channel and a TRPV1 channel, the method comprising contacting the voltage-dependent potassium channel and TRPV1 channel or a cell, a tissue or an organ which expresses the voltage-dependent potassium channel and/or the TRPV1 channel with the compound of claim 1, wherein when said contacting is effected in vivo, the method comprises administering to a subject in need thereof a therapeutically effective amount of the compound.

18. The method of claim 17, wherein modulating said activity of said voltage-dependent potassium channel comprises opening said channel and wherein modulating said activity of said TRPV1 channel comprises inhibiting an activity of said channel.

19. A method of treating a medical condition associated with an activity of a voltage-dependent potassium channel and/or a TRPV1 channel in a subject in need thereof, the method comprising administering to the subject a therapeutically effective amount of the compound of claim 1.

20. The method of claim 19, wherein said medical condition is neuropathic pain, pruritus or tinnitus.
